(19) United States
(12) Patent Application Publication
(10) Pub. No.: US 2021/0047677 A1
(43) Pub. Date: Feb. 18, 2021

(54) NUCLEIC ACID ENRICHMENT WITHIN PARTITIONS

(71) Applicant: 10X GENOMICS, INC., Pleasanton, CA (US)

(72) Inventors: Darisha Jhutty, Saratoga, CA (US); Geoffrey McDermott, Livermore, CA (US)

(21) Appl. No.: 17/003,219

(22) Filed: Aug. 26, 2020

Related U.S. Application Data
(63) Continuation of application No. PCT/US2019/024418, filed on Mar. 27, 2019.
(60) Provisional application No. 62/649,493, filed on Mar. 28, 2018.

(51) Int. Cl. C12Q 1/6806; C12Q 1/6844; C12N 15/10

(52) U.S. Cl. CPC C12Q 1/6806; C12N 15/1096; C12Q 1/6844

(57) ABSTRACT

The present disclosure provides methods and systems for sample processing or analysis. A method for processing a sample may comprise co-partitioning an enzyme and a biological particle comprising a first set of molecules (e.g., RNA molecules comprising one or more mRNA molecules) and a second set of molecules (e.g., RNA molecules other than mRNA molecules) in a partition, lysing or permeabilizing the biological particle to provide access to the first set of molecules and the second set of molecules, and selectively digesting molecules of the second set of molecules with the enzyme to increase a concentration or amount of the first set of molecules relative to the second set of molecules within the partition.

### CROSS REFERENCE

This application is a continuation of International Application No. PCT/US2019/024418, filed Mar. 27, 2019, which claims the benefit of U.S. Provisional Application No. 62/649,493, filed Mar. 28, 2018, which application are entirely incorporated herein by reference.

### BACKGROUND

Samples may be processed for various purposes, such as identification of a type of moiety within the sample. The sample may be a biological sample. The biological samples may be processed for various purposes, such as detection of a disease (e.g., cancer) or identification of a particular species. There are various approaches for processing samples, such as polymerase chain reaction (PCR) and sequencing.

Biological samples may be processed within various reaction environments, such as partitions. Partitions may be wells or droplets. Droplets or wells may be employed to process biological samples in a manner that enables the biological samples to be partitioned and processed separately. For example, such droplets may be fluidically isolated from other droplets, enabling accurate control of respective environments in the droplets.

Partitions and/or biological samples in partitions may be subjected to various processes, such as chemical processes or physical processes. Partitions and/or samples in partitions may be subjected to heating or cooling, or chemical reactions, such as to yield species that may be qualitatively or quantitatively processed.

### SUMMARY

The present disclosure provides methods for use in various sample processing and analysis applications. The methods provided herein may increase a concentration of a first set of molecules within a sample relative to a second set of molecules within the same sample, thereby enriching the first set of molecules within the sample. Such methods may be useful, for example, in controlled analysis and processing of analytes such as biological particles, nucleic acids, and proteins.

In a first aspect, the present disclosure provides a method of processing a sample, the method comprising: (a) providing a biological particle comprising a plurality of ribonucleic acid (RNA) molecules, wherein the plurality of RNA molecules comprises a first set of RNA molecules and a second set of RNA molecules; (b) co-partitioning the biological particle and an RNA enrichment enzyme in a partition among a plurality of partitions, which plurality of partitions is a plurality of droplets or a plurality of wells; (c) in the partition, lysing or permeabilizing the biological particle, thereby providing access to the plurality of RNA molecules of the biological particle; and (d) digesting RNA molecules of the second set of RNA molecules, thereby increasing a concentration or amount of the first set of RNA molecules relative to the second set of RNA molecules within the partition.

In some embodiments, the RNA enrichment enzyme is an exonuclease. In some embodiments, the exonuclease is a 5′-to-3′ exonuclease.

In some embodiments, at least some RNA molecules of the first set of RNA molecules comprise one or more features selected from the group consisting of a 5′ cap structure, an untranslated region (UTR), a 5′ triphosphate moiety, and a 5′ hydroxyl moiety.

In some embodiments, at least some RNA molecules of the second set of RNA molecules comprise a 5′-monophosphate moiety.

In some embodiments, the second set of RNA molecules comprises a ribosomal RNA molecule or a mitochondrial RNA molecule.

In some embodiments, the second set of RNA molecules comprises a ribosomal RNA molecule and a mitochondrial RNA molecule.

In some embodiments, the first set of RNA molecules comprises a messenger RNA (mRNA) molecule and the second set of RNA molecules does not comprise an mRNA molecule. In some embodiments, the method further comprises reverse transcribing the mRNA molecule, thereby generating a complementary deoxyribonucleic acid (cDNA) molecule. In some embodiments, the reverse transcribing occurs in the partition. In some embodiments, the method further comprises releasing the cDNA molecule or a derivative thereof from the partition. In some embodiments, the method further comprises subjecting the cDNA molecule to nucleic acid amplification, thereby generating at least one amplification product of the cDNA molecule. In some embodiments, the nucleic acid amplification occurs in the partition. In some embodiments, the method further comprises releasing or removing the at least one amplification product or a derivative thereof from the partition. In some embodiments, the nucleic acid amplification adds a functional sequence that permits attachment of the at least one amplification product or a derivative thereof to a flow cell of a sequencer. In some embodiments, the method further comprises sequencing the at least one amplification product or a derivative thereof.

In some embodiments, the partition further comprises a reverse transcription enzyme.

In some embodiments, the partition further comprises a bead comprising a plurality of nucleic acid barcode molecules attached thereto, wherein nucleic acid barcode molecules of the plurality of nucleic acid barcode molecules comprise a common barcode sequence. In some embodiments, the plurality of nucleic acid barcode molecules is covalently attached to the bead. In some embodiments, the plurality of nucleic acid barcode molecules is attached to the bead via a disulfide bond. In some embodiments, the bead is a gel bead. In some embodiments, the gel bead comprises a disulfide bond. In some embodiments, the plurality of nucleic acid barcode molecules is releasably attached to the bead. In some embodiments, subsequent to (b), the plurality of nucleic acid barcode molecules is released from the bead. In some embodiments, the plurality of nucleic acid barcode molecules is released from the bead upon exposure to a stimulus. In some embodiments, the stimulus is selected from the group consisting of a thermal stimulus, a photo stimulus, and a chemical stimulus. In some embodiments, the stimulus is a chemical stimulus in the partition. In some embodiments, the chemical stimulus is a reducing agent. In some embodiments, the reducing agent is dithiothreitol. In some embodiments, exposure of the bead to the stimulus results in (i) cleavage of linkages between the plurality of nucleic acid barcode molecules and the bead, and/or (ii) degradation of the bead to release the plurality of nucleic acid barcode molecules from the bead. In some embodiments, the plurality of nucleic acid barcode molecules is released into the partition. In some embodiments, the plurality of nucleic acid barcode molecules comprises at least 100,000 nucleic acid barcode molecules. In some embodiments, the plurality of nucleic acid barcode molecules comprises at least 1,000,000 nucleic acid barcode molecules. In some embodiments, the plurality of nucleic acid barcode molecules comprises at least 10,000,000 nucleic acid barcode molecules. In some embodiments, the plurality of nucleic acid barcode molecules comprises oligo(dT) sequences.

In some embodiments, the method further comprises, subsequent to (d): (i) coupling RNA molecules from the first set of RNA molecules to the bead; and (ii) generating complementary deoxyribonucleic acid (cDNA) molecules from the RNA molecules. In some embodiments, the method further comprises subjecting the cDNA molecules to nucleic acid amplification reactions, thereby generating amplification products of the cDNA molecules. In some embodiments, the method further comprises sequencing the amplification products or derivatives thereof. In some embodiments, the method further comprises, between (i) and (ii), removing or releasing the bead from the partition.

In some embodiments, nucleic acid barcode molecules of the plurality of nucleic acid barcode molecules comprise an identifier sequence that is different from identifier sequences associated with other nucleic acid barcode molecules of the plurality of nucleic acid barcode molecules. In some embodiments, nucleic acid barcode molecules of the plurality of nucleic acid barcode molecules comprise a functional sequence that permits attachment to a flow cell of a sequencer.

In some embodiments, the method further comprises, subsequent to (d), using RNA molecules of the first set of RNA molecules and the nucleic acid barcode molecules of the plurality of nucleic acid barcode molecules to synthesize barcoded RNA molecules. In some embodiments, the synthesizing comprises annealing of the nucleic acid barcode molecules of the plurality of nucleic acid barcode molecules to the RNA molecules of the first set of RNA molecules. In some embodiments, the method further comprises recovering the barcoded RNA molecules from the partition. In some embodiments, the method further comprises sequencing the barcoded RNA molecules or derivatives thereof. In some embodiments, the method further comprises using the barcoded RNA molecules to synthesize barcoded cDNA molecules. In some embodiments, synthesizing the barcoded cDNA molecules comprises reverse transcribing the barcoded RNA molecules. In some embodiments, the method further comprises recovering the barcoded cDNA molecules from the partition. In some embodiments, the method further comprises sequencing the barcoded cDNA molecules or derivatives thereof.

In some embodiments, (c) comprises lysing the biological particle, thereby releasing the plurality of RNA molecules from the biological particle. In some embodiments, (c) comprises permeabilizing the biological particle.

In some embodiments, the biological particle is a cell, a cell nucleus, or a cell bead.

In another aspect, the present disclosure provides a system comprising a plurality of partitions, wherein the plurality of partitions is a plurality of droplets or wells, wherein a partition of the plurality of partitions comprises: a single biological particle comprising a plurality of ribonucleic acid (RNA) molecules, wherein the plurality of RNA molecules comprises one or more messenger RNA (mRNA) molecules; and an enrichment enzyme that is configured to selectively degrade RNA molecules that are not mRNA molecules.

In some embodiments, the biological particle is a cell, a cell nucleus, or a cell bead.

In some embodiments, the plurality of partitions is a plurality of droplets.

In some embodiments, the enrichment enzyme is an exonuclease. In some embodiments, the exonuclease is a 5′-to-3′ exonuclease.

In some embodiments, the partition further comprises a bead comprising a plurality of nucleic acid barcode molecules attached thereto, wherein nucleic acid barcode molecules of the plurality of nucleic acid barcode molecules comprise a common barcode sequence. In some embodiments, the plurality of nucleic acid barcode molecules is covalently attached to the bead. In some embodiments, the plurality of nucleic acid barcode molecules is attached to the bead via a disulfide bond. In some embodiments, the bead is a gel bead. In some embodiments, the gel bead comprises a disulfide bond. In some embodiments, the plurality of nucleic acid barcode molecules is releasably attached to the bead. In some embodiments, subsequent to (b), the plurality of nucleic acid barcode molecules is released from the bead. In some embodiments, the plurality of nucleic acid barcode molecules is capable of being released from the bead upon exposure to a stimulus. In some embodiments, the stimulus is selected from the group consisting of a thermal stimulus, a photo stimulus, and a chemical stimulus. In some embodiments, the stimulus is a chemical stimulus in the partition. In some embodiments, the chemical stimulus is a reducing agent. In some embodiments, the reducing agent is dithiothreitol. In some embodiments, exposure of the bead to the stimulus results in (i) cleavage of linkages between the plurality of nucleic acid barcode molecules and the bead, and/or (ii) degradation of the bead to release the plurality of nucleic acid barcode molecules from the bead. In some embodiments, the plurality of nucleic acid barcode molecules comprises at least 100,000 nucleic acid barcode molecules. In some embodiments, the plurality of nucleic acid barcode molecules comprises at least 1,000,000 nucleic acid barcode molecules. In some embodiments, the plurality of nucleic acid barcode molecules comprises at least 10,000,000 nucleic acid barcode molecules. In some embodiments, the plurality of nucleic acid barcode molecules comprises oligo(dT) sequences.

In some embodiments, the partition further comprises a polymerase. In some embodiments, the partition further comprises a reverse transcriptase.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows an example of a microfluidic channel structure for partitioning individual biological particles.

FIG. 2 shows an example of a microfluidic channel structure for delivering barcode carrying beads to droplets.

FIG. 3 shows an example of a microfluidic channel structure for co-partitioning biological particles and reagents.

FIG. 4 shows an example of a microfluidic channel structure for the controlled partitioning of beads into discrete droplets.

FIG. 5 shows an example of a microfluidic channel structure for increased droplet generation throughput.

FIG. 6 shows another example of a microfluidic channel structure for increased droplet generation throughput.

FIG. 7A shows a cross-section view of another example of a microfluidic channel structure with a geometric feature for controlled partitioning. FIG. 7B shows a perspective view of the channel structure of FIG. 7A.

FIG. 8 illustrates an example of a barcode carrying bead.

FIG. 9 (Panels a-c) shows an exemplary illustration of a selective digestive process carried out within a partition.

FIG. 10 (Panels a-f) shows an exemplary illustration of a selective digestion process carried out within a partition comprising a bead.

FIG. 11 shows an exemplary architecture of a computer system programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

The term “barcode,” as used herein, generally refers to a label, or identifier, that conveys or is capable of conveying information about an analyte. A barcode can be part of an analyte. A barcode can be independent of an analyte. A barcode can be a tag attached to an analyte (e.g., nucleic acid molecule) or a combination of the tag in addition to an endogenous characteristic of the analyte (e.g., size of the analyte or end sequence(s)). A barcode may be unique. Barcodes can have a variety of different formats. For example, barcodes can include: polynucleotide barcodes; random nucleic acid and/or amino acid sequences; and synthetic nucleic acid and/or amino acid sequences. A barcode may be linked to other nucleic acid sequences such as an adapter sequence, a functional sequence, a unique molecular identifier (UMI), a sequencing primer sequence, etc. A barcode may comprise additional nucleic acid sequences such as an adapter sequence, a functional sequence, a UMI, a sequencing primer sequence, etc. A barcode can be attached to an analyte in a reversible or irreversible manner. A barcode can be added to, for example, a fragment of a deoxyribonucleic acid (DNA) or ribonucleic acid (RNA) sample before, during, and/or after sequencing of the sample. Barcodes can allow for identification and/or quantification of individual sequencing-reads.

The term “real time,” as used herein, can refer to a response time of less than about 1 second, a tenth of a second, a hundredth of a second, a millisecond, or less. The response time may be greater than 1 second. In some instances, real time can refer to simultaneous or substantially simultaneous processing, detection or identification.

The term “subject,” as used herein, generally refers to an animal, such as a mammal (e.g., human) or avian (e.g., bird), or other organism, such as a plant. For example, the subject can be a vertebrate, a mammal, a rodent (e.g., a mouse), a primate, a simian or a human. Animals may include, but are not limited to, farm animals, sport animals, and pets. A subject can be a healthy or asymptomatic individual, an individual that has or is suspected of having a disease (e.g., cancer) or a pre-disposition to the disease, and/or an individual that is in need of therapy or suspected of needing therapy. A subject can be a patient. A subject can be a microorganism or microbe (e.g., bacteria, fungi, archaea, viruses).

The term “genome,” as used herein, generally refers to genomic information from a subject, which may be, for example, at least a portion or an entirety of a subject's hereditary information. A genome can be encoded either in DNA or in RNA. A genome can comprise coding regions (e.g., that code for proteins) as well as non-coding regions. A genome can include the sequence of all chromosomes together in an organism. For example, the human genome ordinarily has a total of 46 chromosomes. The sequence of all of these together may constitute a human genome.

The terms “adaptor(s)”, “adapter(s)” and “tag(s)” may be used synonymously. The terms “adapter”, “adapter molecule”, and “adapter nucleic acid sequence” may also be used interchangeably herein. An adaptor or tag can be coupled to a polynucleotide sequence to be “tagged” by any approach, including ligation, hybridization, or other approaches. An adapter molecule, in some cases, may be any useful nucleic acid sequence and may include, for example, a sequencing primer site, a barcode sequence, a transposition site, a restriction site, a unique molecular identifier, a binding sequence, and any/or derivatives, variations, or combinations thereof.

The term “sequencing,” as used herein, generally refers to methods and technologies for determining the sequence of nucleotide bases in one or more polynucleotides. The polynucleotides can be, for example, nucleic acid molecules such as deoxyribonucleic acid (DNA) or ribonucleic acid (RNA), including variants or derivatives thereof (e.g., single stranded DNA). Sequencing can be performed by various systems currently available, such as, without limitation, a sequencing system by Illumina®, Pacific Biosciences (PacBio®), Oxford Nanopore®, or Life Technologies (Ion Torrent®). Alternatively or in addition, sequencing may be performed using nucleic acid amplification, polymerase chain reaction (PCR) (e.g., digital PCR, quantitative PCR, or real time PCR), or isothermal amplification. Such systems may provide a plurality of raw genetic data corresponding to the genetic information of a subject (e.g., human), as generated by the systems from a sample provided by the subject. In some examples, such systems provide sequencing reads (also “reads” herein). A read may include a string of nucleic acid bases corresponding to a sequence of a nucleic acid molecule that has been sequenced. In some situations, systems and methods provided herein may be used with proteomic information.

The term “bead,” as used herein, generally refers to a particle. The bead may be a solid or semi-solid particle. The bead may be a gel bead. The gel bead may include a polymer matrix (e.g., matrix formed by polymerization or cross-linking). The polymer matrix may include one or more polymers (e.g., polymers having different functional groups or repeat units). Polymers in the polymer matrix may be randomly arranged, such as in random copolymers, and/or have ordered structures, such as in block copolymers. Cross-linking can be via covalent, ionic, or inductive, interactions, or physical entanglement. The bead may be a macromolecule. The bead may be formed of nucleic acid molecules bound together. The bead may be formed via covalent or non-covalent assembly of molecules (e.g., macromolecules), such as monomers or polymers. Such polymers or monomers may be natural or synthetic. Such polymers or monomers may be or include, for example, nucleic acid molecules (e.g., DNA or RNA). The bead may be formed of a polymeric material. The bead may be magnetic or non-magnetic. The bead may be rigid. The bead may be flexible and/or compressible. The bead may be disruptable or dissolvable. The bead may be a solid particle (e.g., a metal-based particle including but not limited to iron oxide, gold or silver) covered with a coating comprising one or more polymers. Such coating may be disruptable or dissolvable.

The term “sample,” as used herein, generally refers to a biological sample of a subject. The biological sample may comprise any number of macromolecules, for example, macromolecules of biological particles such as cellular macromolecules. The sample may be a cell sample. The sample may be a cell line or cell culture sample. The sample can include one or more cells. The sample can include one or more microbes. The biological sample may be a nucleic acid sample or protein sample. The biological sample may also be a carbohydrate sample or a lipid sample. The biological sample may be derived from another sample. The sample may be a tissue sample, such as a biopsy, core biopsy, needle aspirate, or fine needle aspirate. The sample may be a fluid sample, such as a blood sample, urine sample, or saliva sample. The sample may be a skin sample. The sample may be a cheek swab. The sample may be a plasma or serum sample. The sample may be a cell-free or cell free sample. A cell-free sample may include extracellular polynucleotides. Extracellular polynucleotides may be isolated from a bodily sample that may be selected from the group consisting of blood, plasma, serum, urine, saliva, mucosal excretions, sputum, stool and tears.

The term “biological particle,” as used herein, generally refers to a discrete biological system derived from a biological sample. The biological particle may be a macromolecule. The biological particle may be a small molecule. The biological particle may be a virus. The biological particle may be a cell or derivative of a cell. The biological particle may be an organelle. The biological particle may be a rare cell from a population of cells. The biological particle may be any type of cell, including without limitation prokaryotic cells, eukaryotic cells, bacterial, fungal, plant, mammalian, or other animal cell type, mycoplasmas, normal tissue cells, tumor cells, or any other cell type, whether derived from single cell or multicellular organisms. The biological particle may be a constituent of a cell. The biological particle may be or may include DNA, RNA, organelles, proteins, or any combination thereof. The biological particle may be or may include a matrix (e.g., a gel or polymer matrix) comprising a cell or one or more constituents from a cell (e.g., cell bead), such as DNA, RNA, organelles, proteins, or any combination thereof, from the cell. The biological particle may be obtained from a tissue of a subject. The biological particle may be a hardened cell. Such hardened cell may or may not include a cell wall or cell membrane. The biological particle may include one or more constituents of a cell, but may not include other constituents of the cell. An example of such constituents is a nucleus or an organelle. A cell may be a live cell. The live cell may be capable of being cultured, for example, being cultured when enclosed in a gel or polymer matrix, or cultured when comprising a gel or polymer matrix.

The term “macromolecular constituent,” as used herein, generally refers to a macromolecule contained within or from a biological particle. The macromolecular constituent may comprise a nucleic acid. In some cases, the biological particle may be a macromolecule. The macromolecular constituent may comprise DNA. The macromolecular constituent may comprise RNA. The RNA may be coding or non-coding. The RNA may be messenger RNA (mRNA), ribosomal RNA (rRNA) or transfer RNA (tRNA), for example. The RNA may be a transcript. The RNA may be small RNA that are less than 200 nucleic acid bases in length, or large RNA that are greater than 200 nucleic acid bases in length. Small RNAs may include 5.8S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA) and small rDNA-derived RNA (srRNA). The RNA may be double-stranded RNA or single-stranded RNA. The RNA may be circular RNA. The macromolecular constituent may comprise a protein. The macromolecular constituent may comprise a peptide. The macromolecular constituent may comprise a polypeptide.

The term “molecular tag,” as used herein, generally refers to a molecule capable of binding to a macromolecular constituent. The molecular tag may bind to the macromolecular constituent with high affinity. The molecular tag may bind to the macromolecular constituent with high specificity. The molecular tag may comprise a nucleotide sequence. The molecular tag may comprise a nucleic acid sequence. The nucleic acid sequence may be at least a portion or an entirety of the molecular tag. The molecular tag may be a nucleic acid molecule or may be part of a nucleic acid molecule. The molecular tag may be an oligonucleotide or a polypeptide. The molecular tag may comprise a DNA aptamer. The molecular tag may be or comprise a primer. The molecular tag may be, or comprise, a protein. The molecular tag may comprise a polypeptide. The molecular tag may be a barcode.

The term “partition,” as used herein, generally, refers to a space or volume that may be suitable to contain one or more species or conduct one or more reactions. A partition may be a physical compartment, such as a droplet or well. The partition may isolate space or volume from another space or volume. The droplet may be a first phase (e.g., aqueous phase) in a second phase (e.g., oil) immiscible with the first phase. The droplet may be a first phase in a second phase that does not phase separate from the first phase, such as, for example, a capsule or liposome in an aqueous phase. A partition may comprise one or more other (inner) partitions. In some cases, a partition may be a virtual compartment that can be defined and identified by an index (e.g., indexed libraries) across multiple and/or remote physical compartments. For example, a physical compartment may comprise a plurality of virtual compartments.

Provided herein are methods that may be used for various sample processing and analysis applications. The method may increase the concentration of a first set of molecules within a sample relative to the concentration of a second set of molecules within the sample, thereby enriching the sample in the first set of molecules. The method may comprise providing a sample comprising a plurality of molecules (e.g., ribonucleic acid [RNA] molecules) comprising a first set of molecules (e.g., a first set of RNA molecules) and a second set of molecules (e.g., a second set of RNA molecules); combining the plurality of molecules with an enzyme; and subjecting the plurality of molecules to conditions suitable for the enzyme to digest molecules of the second set of molecules, thereby increasing a concentration or amount of the first set of molecules relative to the second set of molecules within the sample. The plurality of molecules may be included within a biological particle (e.g., a cell, cell nucleus, or cell bead), and the method may comprise lysing or permeabilizing a biological particle to provide access to the plurality of molecules. The plurality of molecules (e.g., included within a biological particle) may be included within a partition (e.g., a droplet or well). Further processing or analysis of the sample may subsequently take place within or external to the partition. The first and second sets of molecules may comprise ribonucleic acid (RNA) molecules. The first set of RNA molecules may share one or more characteristics such as a sequence, cap structure, or other moiety. In some cases, the first set of RNA molecules may comprise messenger RNA (mRNA) molecules. The present disclosure also provides a partition comprising a biological particle (e.g., a cell, cell nucleus, or cell bead) comprising a plurality of molecules (e.g., RNA molecules), where the plurality of molecules comprises a first set of molecules and a second set of molecules, and an enzyme that is configured to selectively degrade molecules of the second set of molecules.

### Methods of Selective Enrichment Within Partitions

In an aspect, the present disclosure provides a method for use in processing or analyzing a sample. The method may comprise providing a biological particle (e.g., a cell, cell nucleus, or cell bead) comprising a plurality of molecules (e.g., a plurality of ribonucleic acid [RNA] molecules), wherein the plurality of molecules comprises a first set of molecules (e.g., a first set of RNA molecules) and a second set of molecules (e.g., a second set of RNA molecules). The biological particle comprising the plurality of molecules may be co-partitioned with an enzyme in a partition (e.g., a droplet or well). Within the partition, the biological particle may be lysed or permeabilized, thereby providing access to the plurality of molecules of the biological particle. The plurality of molecules within the partition may be subjected to conditions suitable for the enzyme to digest molecules of the second set of molecules of the plurality of molecules, thereby increasing a concentration or amount of the first set of molecules relative to the second set of molecules within the partition.

The plurality of molecules of a sample may comprise a plurality of nucleic acid molecules such as deoxyribonucleic acid (DNA) and ribonucleic acid (RNA) molecules. In some cases, the plurality of molecules derives from a biological particle (e.g., a cell) and comprises RNA molecules. The plurality of RNA molecules may comprise two or more different types of RNA molecules. For example, the plurality of RNA molecules may comprise transfer RNA (tRNA) molecules, ribosomal RNA (rRNA) molecules, mitochondrial RNA (mtRNA) molecules, small nucleolar RNA (snoRNA) molecules, messenger RNA (mRNA) molecules, long non-coding RNAs (lncRNA), and/or other types of RNA molecules. The total RNA derived from a biological particle may comprise a small amount (e.g., less than 10%, such as less than 7%, 6%, 5%, 4%, 3%, 2%, or 1%) of mRNA.

The plurality of molecules of a sample may comprise a first set of molecules that are RNA molecules and a second set of molecules that are also RNA molecules. The first set of RNA molecules may share one or more features. For example, the RNA molecules of the first set of RNA molecules may comprise one or more features selected from the group consisting of a 5′ cap structure, an untranslated region (UTR), a 5′ triphosphate moiety, and a 5′ hydroxyl moiety. One or more RNA molecules of the first set of RNA molecules may also comprise one or more features selected from the group consisting of Kozak sequences, Shine-Dalgarno sequences, coding sequences, and poly(A) sequences (e.g., poly(A) tails). The features shared between RNA molecules of the first set of RNA molecules may be the same or different. For example, RNA molecules of the first set of RNA molecules may all have 5′ cap structures, but may not all have the same 5′ cap structures. In some cases, all RNA molecules of the first set of RNA molecules may share one or more features, and the one or more features may be the same or substantially the same. For example, all RNA molecules of the first set of RNA molecules may share the same 5′ cap structure. RNA molecules of the first set of RNA molecules may comprise multiple features described above, such as both a 5′ cap structure and one or more untranslated regions. For example, RNA molecules of the first set of RNA molecules may comprise a 5′ cap structure, a 5′ UTR, and a 3′ UTR. In some cases, RNA molecules of the first set of RNA molecules may comprise a 5′ cap structure, a 5′ UTR, a coding sequence, a 3′ UTR, and a polyA tail.

Features of RNA molecules may have any useful characteristics. A 5′ cap structure may comprise one or more nucleoside moieties joined by a linker such as a triphosphate (ppp) linker. A 5′ cap structure may comprise naturally occurring nucleoside and/or non-naturally occurring (e.g., modified) nucleosides. For example, a 5′ cap structure may comprise a guanine moiety or a modified (e.g., alkylated, reduced, or oxidized) guanine moiety such as a 7-methylguanylate (m⁷G) cap. Examples of 5′ cap structures include, but are not limited to, m⁷GpppG, m⁷Gpppm⁷G, m⁷GpppA, m⁷GpppC, GpppG, m^{2,7}GpppG, m^{2,2,7}GpppG, and anti-reverse cap analogs such as m^{7,2′Ome}GpppG, m^{7,2′d}GpppG, m^{7,3′Ome}GpppG, and m^{7,3′d}GpppG. An untranslated region (UTR) may be a 5′ UTR or a 3′ UTR. A UTR may include any number of nucleotides. For example, a UTR may comprise at least 3, 5, 7, 10, 20, 30, 40, 50, 60, 70, 80, 90, 100, or more nucleotides. In some cases, a UTR may comprise fewer than 20 nucleotides. In other cases, a UTR may comprise at least 100 nucleotides, such as more than 200, 300, 400, 500, 600, 700, 800, 900, or 1000 nucleotides. Similarly, a coding sequence may include any number of nucleotides, such as at least 3, 5, 10, 20, 30, 40, 50, 60, 70, 80, 90, 100, or more nucleotides. A UTR, coding sequence, or other sequence of an RNA molecule or a collection of RNA molecules (e.g., the first set of RNA molecules of the plurality of RNA molecules of a cell) may have any nucleotide or base content or arrangement. For example, a sequence of an RNA molecule or a collection of RNA molecules may comprise any number or concentration of guanine, cytosine, uracil, and adenine bases. An RNA molecule or a collection of RNA molecules may also include non-naturally occurring (e.g., modified) nucleosides. A modified nucleoside may comprise one or more modifications (e.g., alkylations, hydroxylation, oxidation, or other modification) in its nucleobase and/or sugar moieties. In some embodiments, the RNA molecules having one or more of the features described herein are messenger RNA (mRNA) molecules.

The first set of RNA molecules of the plurality of molecules of a sample may comprise mRNA molecules. In some cases, all of the RNA molecules of the first set of RNA molecules may be mRNA molecules. Alternatively, the first set of RNA molecules may comprise one or more mRNA molecules as well as one or more other RNA molecules comprising one or more features selected from the group consisting of a 5′ cap structure, a UTR, a 5′ triphosphate moiety, and a 5′ hydroxyl moiety. In some cases, the first set of RNA molecules may comprise one or more ribosomal RNA (rRNA) molecules and/or one or more transfer RNA (tRNA) molecules. In some cases, one or more of the RNA molecules of the first set may be a microRNA, a long non-coding RNA, a small nucleolar RNA, a circular RNA, small RNA, RNA isoforms, snoRNA, piRNA, or any RNA molecule that may contribute to cellular phenotype.

The second set of RNA molecules of the plurality of molecules of a sample may also share one or more features such as those described herein. In some cases, RNA molecules of the second set of RNA molecules may not comprise a feature selected from the group consisting of a 5′ cap structure, a UTR, a 5′ triphosphate moiety, and a 5′ hydroxyl moiety. The second set of RNA molecules may comprise one or more rRNA molecules and/or one or more mitochondrial RNA (mtRNA) molecules. In some cases, the second set of RNA molecules may not include an mRNA molecule.

The plurality of molecules (e.g., RNA molecules) of a sample may be included within one or more biological particles (e.g., cells, cell beads, or cell nuclei). For example, the sample may comprise a cell comprising a plurality of RNA molecules comprising a first set of RNA molecules (e.g., as described herein) and a second set of RNA molecules (e.g., as described herein). The cell may be, for example, a human cell, an animal cell, or a plant cell. In some cases, the cell may be derived from a tissue or fluid, as described herein. The cell may be a prokaryotic cell or a eukaryotic cell. The cell may be a lymphocyte such as a B cell or T cell.

A biological particle (e.g., cell, cell nucleus, or cell bead) may be fixed, embedded, and/or embalmed. Fixation may preserve one or more morphological features of a cell and may provide a rigid cell. For example, fixation may preserve a size of a cell and/or relative locations of cellular components within a cell. In some cases, fixation may comprise dehydration of the cell and/or may result in shrinkage or size reduction of the cell. Fixation may be achieved through the use of a fixative such as an aldehyde (e.g., formaldehyde (such as formalin), paraformaldehyde, or glutaraldehyde), alcohol (e.g., ethanol or methanol), acetic acid, a ketone (e.g., acetone), osmium tetraoxide, potassium dichromate, chronic acid, potassium permanganate, Zenker's fixative, picrates, Hepes-glutamic acid buffer-mediated organic solvent protection effect (HOPE), a labile group such as dithiobis(succinimidyl propionate), disuccinimidyl suberate (DSS), dimethylsuberimidate (DMS), formalin, and dimethyladipimidate (DMA), dithio-bis(-succinimidyl propionate) (DSP), disuccinimidyl tartrate (DST), or ethylene glycol bis(succinimidyl succinate) (EGS). In some cases, a fixative may be a cross-linking agent such as a photocleavable crosslinker or an aldehyde (e.g., formaldehyde or glutaraldehyde). In some cases, a combination of fixatives may be used. For example, a first fixative may be used to change a first characteristic of a cell (e.g., cell size) and a second fixative may be used to change a second characteristic of the cell (e.g., fluidity or rigidity). For example, one or more fixatives may be used to reduce the size of a cell in one or more dimensions. The first and second fixatives may be used at the same or different times. In some cases, a fixative may be used to form a gel matrix comprising the cell. Gel matrix formation may cause sufficient force on a cell, causing it to lyse. Alternatively or in addition, a cell may be embalmed (e.g., using an embalming fluid) and/or embedded (e.g., using an embedding media). An embedded cell may be hardened using, for example, a resin or wax (e.g., paraffin wax).

Access to a plurality of molecules included in a biological particle (e.g., a cell, cell nucleus, or cell bead) may be provided by lysing or permeabilizing the biological particle. Lysing a cell may release the plurality of RNA molecules contained therein from a cell. A cell may be lysed using a lysis agent such as a bioactive agent. A bioactive agent useful for lysing a cell may be, for example, an enzyme (e.g., as described herein). An enzyme used to lyse a cell may or may not be capable of carrying out additional actions such as degrading one or more RNA molecules. Alternatively, an ionic, zwitterionic, or non-ionic surfactant may be used to lyse a cell. Examples of surfactants include, but are not limited to, TritonX-100, CHAPS, Tween 20, sarcosyl, or sodium dodecyl sulfate. Cell lysis may also be achieved using a cellular disruption method such as an electroporation or a thermal, acoustic, or mechanical disruption method, or through use of osmotic pressure, e.g., using a hypotonic lysis buffer. In some cases, a cell may be lysed via formation of a gel matrix (e.g., within the cell, such as by use of a cross-linking agent). Alternatively, a cell may be permeabilized to provide access to a plurality of nucleic acid molecules included therein. Permeabilization may involve partially or completely dissolving or disrupting a cell membrane or a portion thereof. Permeabilization may be achieved by, for example, contacting a cell membrane with an organic solvent or a detergent such as Triton X-100 or NP-40.

A biological particle (e.g., a cell, cell nucleus, or cell bead) may be partitioned within a partition such as a well or droplet, e.g., as described herein. One or more reagents may be co-partitioned with a biological particle. For example, a biological particle may be co-partitioned with one or more reagents selected from the group consisting of lysis agents or buffers, permeabilizing agents, enzymes (e.g., enzymes capable of digesting one or more nucleic acid (e.g., RNA) molecules, extending one or more nucleic acid molecules, ligating one or more nucleic acid molecules, reverse transcribing an RNA molecule, tagmenting (e.g., fragmenting and in some cases, adding a tag to) nucleic acid molecules, permeabilizing or lysing a cell, or carrying out other actions), fluorophores, oligonucleotides, primers, barcodes, nucleic acid barcode molecules (e.g., nucleic acid barcode molecules comprising one or more barcode sequences), buffers, deoxynucleotide triphosphates, detergents, reducing agents, chelating agents, oxidizing agents, nanoparticles, beads, and antibodies. In some cases, a biological particle may be co-partitioned with one or more reagents selected from the group consisting of temperature-sensitive enzymes, pH-sensitive enzymes, light-sensitive enzymes, reverse transcriptases, proteases, ligase, polymerases, restriction enzymes, nucleases, protease inhibitors, exonucleases, transposases, and nuclease inhibitors. For example, a biological particle may be co-partitioned with a reverse transcriptase and nucleotide molecules. Alternatively or in addition, a biological particle may be co-partitioned with an enzyme such as an exonuclease capable of digesting molecules of the plurality of molecules included on or within the biological particle. Partitioning a biological particle and one or more reagents may comprise flowing a first phase comprising an aqueous fluid, the biological particle, and the one or more reagents and a second phase comprising a fluid that is immiscible with the aqueous fluid toward a junction. Upon interaction of the first and second phases, a discrete droplet of the first phase comprising the biological particle and the one or more reagents may be formed. In some cases, the partition may comprise a single biological particle. The biological particle (e.g., cell) may be lysed or permeabilized within the partition (e.g., droplet) to provide access to the plurality of molecules of the biological particle. Accordingly, molecules originating from the same biological particle may be isolated within the same partition.

A biological particle comprising a plurality of molecules (e.g., RNA molecules) may be co-partitioned with an enzyme. Alternatively, the plurality of molecules inside or released from the biological particle may be brought into contact with the enzyme outside a partition. The enzyme may be capable of selectively digesting one or more molecules of the plurality of molecules of the biological particle. The enzyme may be an exonuclease such as a 5′-to-3′ exonuclease (e.g., a 5′-phosphate dependent exonuclease). The activity of such an enzyme may be modulated by an agent such as an inorganic ion such as a magnesium ion. The enzyme may digest molecules having particular features. For example, the enzyme may digest RNA molecules having a 5′-monophosphate moiety. Such molecules include prokaryotic 16S and 23S rRNA and eukaryotic 18S and 28S rRNA. Similarly, the enzyme may not digest molecules having other particular features. For example, the enzyme may not digest RNA molecules having a 5′ cap structure (e.g., eukaryotic RNA with a 5′ cap structure), a 5′-triphosphate moiety (e.g., prokaryotic mRNA with a 5-triphosphate moiety), or a 5′-hydroxyl moiety (e.g., degraded RNA with a 5′-hydroxyl moiety). The enzyme may not be capable of digesting, for example, 5S rRNA molecules, which have a 5′-triphosphate moiety, and tRNA, which has an inaccessible 5′-monophosphate moiety. Such species may be removable from a sample using, for example, selective precipitation with lithium chloride. The enzyme may not be inhibited by proteinaceous RNase inhibitors. Such an enzyme may allow for selective digestion of RNA molecules without the use of columns, beads, or immobilized oligo(dT) matrices. Accordingly, such an enzyme may be capable of increasing the concentration of a first set of RNA molecules relative to a second set of RNA molecules that is at least partially digestable by the enzyme. For example, the enzyme may be capable of selectively digesting RNA molecules that are not mRNA molecules, thereby enriching mRNA molecules within a container or partition. An example of such an enzyme is the Terminator Exonuclease (Epicentre® Biotechnologies). Digestion of RNA molecules by an enzyme may take place within a partition (e.g., as described herein), or at any convenient step or location. In some cases, digestion of RNA molecules by an enzyme may occur outside a partition (e.g., in bulk) and may occur prior to or following partitioning.

The method of the present disclosure may comprise subjecting the plurality of RNA molecules of a biological particle (e.g., within a partition) to conditions suitable for the enzyme to digest RNA molecules of the second set of RNA molecules, thereby increasing a relative concentration or amount of the first set of RNA molecules relative to the second set of RNA molecules (e.g., within a partition). Subjecting RNA molecules to conditions suitable for enzymatic digestion may comprise incubating the partition (e.g., droplet or well) or a sample comprising the partition (e.g., a container including an emulsion of droplets) at a particular temperature for a period of time. An incubation temperature may be, for example, at least about 25° C., 30° C., 35° C., 40° C., 45° C., 50° C., 55° C., 60° C., or higher. A partition or a sample comprising the partition may be incubated for at least 5, 10, 15, 20, 25, 30, 35, 40, 45, 50, 55, 60, or more minutes. For example, a partition or a sample comprising the partition may be incubated at about 30° C. for about 30-60 minutes. In some cases, a partition or a sample comprising the partition may optionally be incubated again. For example, the partition or a sample comprising the partition may be incubated again at about 53° C. for about 30-60 minutes. The second incubation period may allow for further digestion of RNA molecules within the partition and/or may facilitate other processes such as, for example, reverse transcription of mRNA molecules to form cDNA molecules (e.g., as described herein). Subjecting RNA molecules to conditions suitable for enzymatic digestion may further comprise subjecting RNA molecules to a given pH. For example, a buffer provided in a partition comprising a biological particle (e.g., a cell, cell nucleus, or cell bead) comprising a plurality of RNA molecules may be used to adjust the pH of the partition to a desired value such as at least 5.5, 6, 6.5, 7, 7.5, or 8. The concentration of the enzyme may also be altered. For example, the concentration of the enzyme may be determined based on the size or other characteristics of the partition or biological particle.

Digestion of all or a portion of a second set of molecules of a plurality of molecules of a biological particle (e.g., within a partition) may increase the relative concentration or amount of a first set of molecules of the plurality of molecules (e.g., relative to the total set of the first and second sets of molecules). For example, digestion of all or a portion of a second set of RNA molecules in a partition may increase the relative concentration of a first set of RNA molecules. The relative concentration of the first set of RNA molecules may increase by at least 5%, 10%, 15%, 20%, 30%, 35%, 40%, 45%, 50%, 55%, 60%, 65%, 70%, 75%, 80%, 85%, 90%, 95%, 100%, or more. In some cases, the relative concentration of the first set of RNA molecules may increase by at least twofold, such as at least threefold, fourfold, fivefold, tenfold, or more. Correspondingly, the relative concentration of the second set of RNA molecules may decrease by at least 5%, 10%, 15%, 20%, 30%, 35%, 40%, 45%, 50%, 55%, 60%, 65%, 70%, 75%, 80%, 85%, 90%, 95%, or 100%. In some cases, all of the molecules of the second set of molecules may be digested. Alternatively, at least 1%, 2%, 5%, 10%, 15%, 20%, 30%, 35%, 40%, 45%, 50%, 55%, 60%, 65%, 70%, 75%, 80%, 85%, 90%, 95%, 96%, 97%, 98%, or 99% of the molecules of the second set of molecules may be digested. In one example, the plurality of RNA molecules of a biological particle (e.g., a cell, cell nucleus, or cell bead) comprises a first set of RNA molecules comprising one or more mRNA molecules and a second set of RNA molecules that does not comprise an mRNA molecule, and selective digestion of one or more RNA molecules of the second set of RNA molecules results in a tenfold enrichment of the first set of RNA molecules relative to the second set of RNA molecules. The enrichment of the first set of RNA molecules may correspond to a greater than 40% reduction of the second set of RNA molecules (e.g., the combined fraction of rRNA and tRNA in the plurality of molecules).

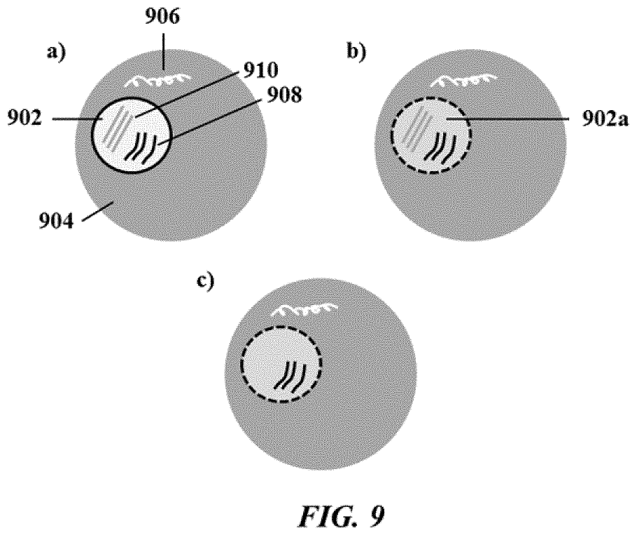

FIG. 9 shows a schematic of a selective digestive process carried out within a partition. Panel (a) of FIG. 9) shows a biological particle (e.g., a cell, cell nucleus, or cell bead) 902 and enzyme 906 contained within partition 904. Partition 904 may be a droplet or well. Biological particle 902 contains a plurality of RNA molecules comprising a first set of RNA molecules 908 and a second set of RNA molecules 910. Panel (b) of FIG. 9 shows a permeabilized biological particle 902a. Biological particle 902 may be permeabilized using a reagent such as a detergent contained within partition 904 or may be permeabilized prior to partitioning. Panel (c) of FIG. 9 shows permeabilized biological particle 902a after selective digestion of second set of RNA molecules 910 by enzyme 906. After digestion of second set of RNA molecules 910, the concentration and amount of first set of RNA molecules 908 in permeabilized cell 902a and partition 904 is increased relative to the second set of RNA molecules 910.

Subsequent to selective digestion of RNA molecules of a second set of RNA molecules (e.g., within a partition such as a well or droplet) and the corresponding relative increase in concentration or amount of a first set of RNA molecules, one or more RNA molecules of the first set of RNA molecules may undergo further processing. For example, one or more RNA molecules of the first set of RNA molecules may be used to synthesize one or more barcoded nucleic acid molecules (e.g., via a primer extension and/or nucleic acid amplification process). Alternatively or in addition, one or more RNA molecules of the first set of RNA molecules may be subjected to conditions suitable for carrying out reverse transcription. For example, in a partition comprising a reverse transcriptase and nucleotide molecules, mRNA molecules of the first set of RNA molecules may be reserve transcribed to generate one or more complementary deoxyribonucleic acid (cDNA) molecules (e.g., as described herein). cDNA molecules, or the partition in which they are contained, may then be subjected to conditions suitable to synthesize one or more nucleic acid molecules (e.g., barcoded nucleic acid molecules). For example, one or more primer extension and/or nucleic acid amplification reactions may be performed. Such reactions may comprise the use of one or more primers and one or more polymerases. In some cases, a primer extension and/or amplification reaction may comprise the use of a splint sequence that is configured to bind to a sequence of a target molecule (e.g., an RNA molecule of the first set of RNA molecules) and to a sequence of a primer molecule or nucleic acid barcode molecule. Amplification reactions may be, for example, polymerase chain reactions (PCR). Where digestion of RNA molecules takes place within a partition (e.g., a well or droplet), RNA molecules of the first set of RNA molecules may be released from the partition prior to any subsequent processing. For example, reverse transcription, amplification, and any other processing may take place outside of a partition, such as in a bulk container. Alternatively, one or more additional processes may be carried out within a partition. For example, RNA molecules of the first set of RNA molecules may undergo reverse transcription within the partition to generate cDNA molecules, which may then be released from the partition (e.g., by breaking an emulsion). The cDNA molecules may comprise barcode sequences (e.g., as described herein). Subsequent to release from the partition, the cDNA molecules (e.g., barcoded cDNA molecules) may be used to synthesize a plurality of nucleic acid molecules, which plurality of nucleic acid molecules comprise sequences of the cDNA molecules or complements thereof (e.g., including barcode sequences of complements thereof). The plurality of nucleic acid molecules may be a plurality of amplified products. The plurality of nucleic acid molecules may be subjected to further analysis such as nucleic acid sequencing. In another example, RNA molecules of the first set of RNA molecules may undergo reverse transcription within the partition to generate cDNA molecules, which cDNA molecules may comprise barcode sequences (e.g., as described herein). Within the partition, the cDNA molecules (e.g., barcoded cDNA molecules) may be used to synthesize a plurality of nucleic acid molecules, which plurality of nucleic acid molecules comprise sequences of the cDNA molecules or complements thereof (e.g., including barcode sequences of complements thereof). The resultant plurality of nucleic acid molecules may be a plurality of amplified products. The plurality of nucleic acid molecules (e.g., amplified products) may then be released from the partition for further analysis such as nucleic acid sequencing (e.g., by breaking an emulsion). In some cases, RNA molecules of the first set of RNA molecules may be captured by a bead (e.g., a magnetic particle or other solid particle) included within the partition. The particle having RNA molecules attached thereto may then be released from the partition for subsequent processing steps including, for example, reverse transcription to generate cDNA molecules and/or primer extension and/or amplification reactions to generate nucleic acid molecules comprising sequences of the RNA molecules or complements thereof. In some cases, the bead may be an affinity bead for which certain nucleic acid molecules have a higher affinity. For example, the affinity bead may capture RNA molecules of the first set of RNA molecules but not other RNA molecules of the plurality of RNA molecules of the initial biological particle included in the partition. In some cases, RNA molecules of the plurality of RNA molecules included in the partition may undergo processing (e.g., before or subsequent to a digestion/enrichment process) to induce or increase affinity of at least a subset of the RNA molecules for the affinity bead. For example, at least a subset of the RNA molecules may be processed to include a label or sequence that is configured to attach to the affinity bead or to a linker moiety that is attached to or configured to attach to the affinity bead.

A partition (e.g., a well or droplet) comprising a biological particle (e.g., a cell, cell nucleus, or cell bead) may further comprise a bead (e.g., as described herein). The bead may be a gel bead. The bead may comprise a plurality of nucleic acid barcode molecules (e.g., nucleic acid molecules each comprising one or more barcode sequences, as described herein). The bead may comprise at least 10,000 nucleic acid barcode molecules attached thereto. For example, the bead may comprise at least 100,000, 1,000,000, or 10,000,000 nucleic acid barcode molecules attached thereto. The plurality of nucleic acid barcode molecules may be releasably attached to the bead. The plurality of nucleic acid barcode molecules may be attached to the bead via a plurality of labile moieties. The plurality of nucleic acid barcode molecules may be releasable from the bead upon application of a stimulus. Such a stimulus may be selected from the group consisting of a thermal stimulus, a photo stimulus, and a chemical stimulus. For example, the stimulus may be a reducing agent such as dithiothreitol Application of a stimulus may result in one or more of (i) cleavage of a linkage between nucleic acid barcode molecules of the plurality of nucleic acid barcode molecules and the bead, and (ii) degradation or dissolution of the bead to release nucleic acid barcode molecules of the plurality of nucleic acid barcode molecules from the bead.

A nucleic acid barcode molecule (e.g., a nucleic acid barcode molecule coupled to a bead) may comprise any useful structure and combination of sequences. A nucleic acid barcode molecule may comprise DNA or RNA. In some cases, a nucleic acid barcode molecule may comprise both RNA and DNA. A nucleic acid barcode molecule may comprise canonical nucleotides (e.g., A, T, C, G, and U) and/or one or more nucleotide analogs. For example, a nucleic acid barcode molecule may comprise one or more modifications, combinations, derivatives, or variations of nucleotides and/or nucleic acid molecules (e.g., as described elsewhere herein). A nucleic acid barcode molecule may be single-stranded or double-stranded. Alternatively, a nucleic acid barcode molecule may be partially double-stranded and/or partially single-stranded. For example, a first strand of a nucleic acid barcode molecule may be coupled to a bead and comprise a first sequence, and the second strand of the nucleic acid barcode molecule may comprise a second sequence that is hybridized to the first sequence and may not be directly coupled to the bead. In some cases, the nucleic acid barcode molecule may comprise an overhang sequence (e.g., disposed at an end distal to the bead to which the nucleic acid barcode molecule is coupled) that is configured to hybridize to a first sequence of a splint molecule. The splint molecule may comprise a second sequence that is configured to hybridize to a nucleic acid molecule (e.g., mRNA or cDNA molecule) or a molecule hybridized or ligated thereto (e.g., an adapter nucleic acid molecule). In some cases, a nucleic acid barcode molecule may be configured to ligate to a nucleic acid molecule (e.g., mRNA or cDNA molecule) or to a molecule hybridized or ligated thereto (e.g., an adapter nucleic acid molecule). For example, an enzyme (e.g., ligase) or click chemistry approach may be used to ligate a nucleic acid barcode molecule to another molecule to provide a barcoded nucleic acid molecule. In such an instance, amplification and/or primer extension may not be necessary to barcode a nucleic acid molecule (e.g., mRNA or cDNA molecule). A barcode sequence of a nucleic acid barcode molecule may comprise one or more segments. In some cases, a barcode sequence may be generated using a combinatorial assembly method such as a split-pool approach. Alternatively or in addition, a set of molecules (e.g. mRNA) molecules within a cell or cell bead may be barcoded in a combinatorial manner (e.g., via a split-pool approach) to generate combinatorially barcoded cells.

A partition comprising a biological particle comprising a plurality of molecules (e.g., a first set of molecules and a second set of molecules, as described herein) may comprise a plurality of nucleic acid barcode molecules. The plurality of nucleic acid barcode molecules may be coupled to a single bead. Alternatively, the plurality of nucleic acid barcode molecules may be coupled to multiple beads, such as two beads, included within the partition. Each nucleic acid barcode molecule of the plurality of nucleic acid barcode molecules may comprise a common barcode sequence. In some cases, the common barcode sequences may be unique to the partition, such that no two partitions among a plurality of partitions each comprising a biological particle and a plurality of nucleic acid barcode molecules comprise the same common barcode sequence. Each nucleic acid barcode molecule of the plurality of nucleic acid barcode molecules of a partition may also comprise an additional barcode sequence such as a unique molecular identifier sequence. In some cases, each nucleic acid barcode molecule of the plurality of nucleic acid barcode molecules may comprise a common barcode sequence and one or more additional common sequences, such as one or more sequencing primers, flow cell sequences, overhang sequences, promoter sequences, primer annealing sequences, immobilization sequences, or other functional sequences. In some cases, the plurality of nucleic acid barcode molecules of a partition comprises a first set of nucleic acid barcode molecules and a second set of nucleic acid barcode molecules. The nucleic acid barcode molecules of the first set of nucleic acid barcode molecules and nucleic acid barcode molecules of the second set of nucleic acid barcode molecules may comprise a common barcode sequences and one or more different functional sequences. For example, nucleic acid barcode molecules of the first set of nucleic acid barcode molecules may comprise a first functional sequence and nucleic acid barcode molecules of the second set of nucleic acid barcode molecules may comprise a second functional sequence that is different than the first functional sequence. The first set of nucleic acid barcode molecules and the second set of nucleic acid barcode molecules may be attached to the same or different beads. Details of nucleic acid barcode molecules and their generation can be found, for example, in PCT/US2018/061391.

A nucleic acid barcode molecules may be used to generate a barcoded nucleic acid molecule (e.g., barcoded RNA, barcoded cDNA, etc.). Barcoding may take place at any convenient time during nucleic acid processing. For example, barcoding may be performed on the first set of molecules (e.g., mRNA) subsequent to their enrichment within the partition (e.g., as described herein). In some cases, barcoding may be performed on the plurality of molecules within the partition prior to a digestion/enrichment process. For example, the biological particle comprising the plurality of molecules may be provided within the partition and the plurality of molecules may be barcoded. The barcoded plurality of molecules may be released from the partition and then subjected to a digestion/enrichment process (e.g., as described herein). Alternatively, the barcoded plurality of molecules may be subjected to a digestion/enrichment process (e.g., as described herein) within the partition. In some cases, barcoding may be performed on derivatives of the plurality of molecules or a subset thereof of a biological particle within a partition. For example, barcoding may be performed on cDNA molecules synthesized from the first set of molecules (e.g., prior or subsequent to a digestion/enrichment process). In such cases, a first set of molecules (e.g., RNA molecules) may be reverse transcribed, and the resultant products (e.g., cDNA molecules) may be barcoded. One or more of these processes may occur in a partition or in a bulk container.

The plurality of nucleic acid barcode molecules attached to a bead within a partition (e.g., a well or droplet) comprising a plurality of molecules comprising a first set of molecules (e.g., RNA molecules) and a second set of molecules (e.g., RNA molecules) may be suitable for barcoding the first set of molecules of the plurality of molecules of the biological particle, and/or corresponding cDNA molecules generated through reverse transcription. In some cases, the plurality of nucleic acid barcode molecules attached to the bead may comprise a common barcode sequence. The plurality of nucleic acid barcode molecules may also comprise one or more functional sequences selected from the group consisting of a primer sequence, a primer annealing sequence, and an immobilization sequence. Nucleic acid barcode molecules comprising a primer sequence may be useful in the amplification of one or more nucleic acid molecules (e.g., RNA or cDNA molecules). For example, one or more cDNA molecules generated from reverse transcription within a partition (e.g., as described herein) may be subjected to conditions suitable for performing amplification reactions (e.g., PCR), thereby generating amplification products, where the amplification reactions comprise annealing a primer sequence of one or more of the nucleic acid barcode molecules to cDNA molecules. Prior to performing the amplification reactions, nucleic acid barcode molecules of the plurality of nucleic acid barcode molecules may be released from the bead upon application of a stimulus to enhance the probability of interaction between the nucleic acid barcode molecules and the cDNA molecules.

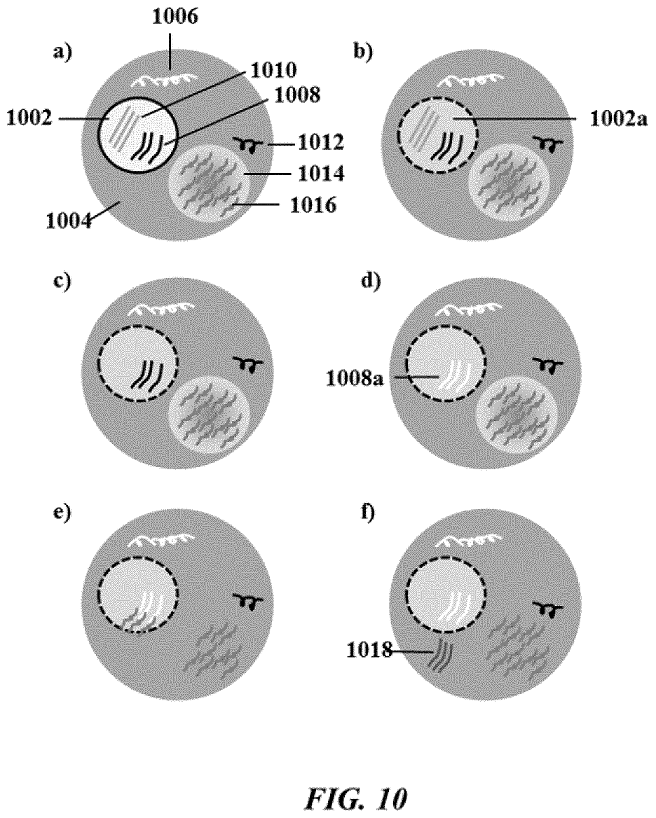

FIG. 10 show a schematic of a selective digestion process carried out within a partition with a bead. Panel (a) of FIG. 10 shows biological particle (e.g., cell, cell nucleus, or cell bead) 1002 and enzyme 1006 contained within partition 1004. Partition 1004 may be a droplet or well. In addition to biological particle 1002 and enzyme 1006, partition 1004 comprises reverse transcriptase 1012 and bead 1014 having a plurality of nucleic acid barcode molecules 1016 attached thereto. Biological particle 1002 contains a plurality of RNA molecules comprising a first set of RNA molecules 1008 and a second set of RNA molecules 1010. Panel (b) of FIG. 10 shows a permeabilized biological particle 1002a. Biological particle 1002 may be permeabilized using a reagent such as a detergent contained within partition 1004 or permeabilized prior to partitioning. Panel (c) of FIG. 10 shows permeabilized biological particle 1002a after selective digestion of second set of RNA molecules 1010 by enzyme 1006. After digestion of second set of RNA molecules 1010, the concentration and amount of first set of RNA molecules 1008 in permeabilized biological particle 1002a and partition 1004 is increased relative to second set of RNA molecules 1010. Panel (d) of FIG. 10 shows first set of RNA molecules 1008a reverse transcribed by reverse transcriptase 1012. Panel (e) of FIG. 10 shows reversed transcribed RNA molecules 1008a annealing to primer sequences of nucleic acid barcode molecules 1016 of bead 1014. Nucleic acid barcode molecules 1016 are released from bead 1014 by application of a stimulus. In some cases, application of a stimulus causes all or a portion of bead 1014 to degrade or dissolve. Panel (f) of FIG. 10 shows amplification products 1018 produced by amplification reactions involving reversed transcribed RNA molecules 1008a and nucleic acid barcode molecules 1016. Amplification products 1018 each include a common nucleic acid barcode sequence of nucleic acid barcode molecules 1016. Repetition of the processes illustrated in FIG. 10 may result in a plurality of partitions each including amplification products sharing a common nucleic acid barcode sequence, where each partition includes a different nucleic acid barcode sequence. In some examples, first set of RNA molecules 1008 are released from partition 1004 prior to undergoing reverse transcription, and reverse transcription and amplification may be performed outside of a partition. In other examples, first set of RNA molecules 1008 are reverse transcribed within partition 1004 and released prior to undergoing amplification, and amplification may be performed outside of a partition.

The methods described herein may comprise the use of a cell bead. Analytes such as molecules derived from a cell (e.g., nucleic acid molecules, metabolites, proteins, and other molecules) may be comprised within a cell bead matrix, attached to a cell bead, and/or attached to a particle (e.g., magnetic particle) within a cell bead. A cell bead may be generated by, for example, flowing a first fluid comprising polymeric or gel precursors and a second fluid comprising cell or virus reagents (e.g., via action of an applied force, such as negative pressure via a vacuum or positive pressure via a pump) from reservoirs to a first channel junction at which point they combine to form an aqueous stream. This aqueous stream may then be flowed to a second channel junction to which an immiscible fluid such as oil may be provided, resulting in the generation of a suspension of aqueous droplets in the oil. The droplets may then be subjected to conditions suitable to polymerize or gel the polymeric or gel precursors in the droplets to generate cell beads that encapsulate the cell or virus reagents. The cell may be alive or dead. In some cases, the cell may be fixed, and in some instances, permeabilized, prior to generating the cell bead. Solvent exchange may then be used to resuspend the cell beads in an aqueous phase and subjected to further analysis. In some cases, cell beads may be repartitioned (e.g., as described herein) to generate a partition (e.g., a droplet or well) including a cell bead. In some cases, a cell bead may be partitioned together with a bead comprising a plurality of nucleic acid barcode molecules (e.g., as described herein). Cell beads may be useful for hindering diffusion of larger molecules such as nucleic acid molecules and proteins within a partition. Like the beads comprising nucleic acid barcode molecules described elsewhere herein, the contents of cell beads may be released upon application of a stimulus. For example, a cell bead may be completely or partially dissolved or degraded within a partition to release trapped constituents of a biological particle to the interior of the partition. The freed cellular components may then interact with other components of the particle including nucleic acid barcode molecules of a gel bead, an exonuclease, and/or other components.

In an example, a cell comprising a first set of RNA molecules and a second set of RNA molecules is partitioned with polymeric or gel precursors to form a first droplet. The first droplet is subjected to conditions suitable to polymerize the polymeric or gel precursors to form a cell bead. The resultant cell bead is then partitioned in an aqueous droplet with various reagents including an exonuclease and a gel bead comprising a plurality of nucleic acid barcode molecules attached thereto. A stimulus may be applied to partially degrade or dissolve the cell bead to release RNA molecules included therein. In some cases, the stimulus may also release nucleic acid barcode molecules from the gel bead. In some cases, the stimulus may be a chemical reagent (e.g., a reducing agent) and may be co-partitioned with the cell bead and the gel bead. RNA molecules of the second set of RNA molecules may then be selectively digested by the exonuclease to enrich the first set of RNA molecules within the partition. RNA molecules of the first set of RNA molecules may then undergo subsequent analysis and processing such as reverse transcription, amplification, and sequencing (e.g., as described herein).

The methods described herein may be applied to a single biological particle (e.g., a single cell, cell nucleus, or cell bead) or a plurality of biological particles (e.g., a plurality of cells, cell nuclei, or cell beads). A method of processing a sample comprising a plurality of biological particles may comprise providing the plurality of biological particles, where each biological particle of the plurality of biological particles comprises a plurality of RNA molecules, where the plurality of RNA molecules of each biological particle comprises a first set of RNA molecules and a second set of RNA molecules. The plurality of biological particles may be co-partitioned with a plurality of enzymes (e.g., as described herein) into a plurality of separate partitions, such that each partition of a plurality of different partitions of the plurality of separate partitions contains a single biological particle and an enzyme. In some cases, some partitions of the plurality of separate partitions may not include a biological particle. Each enzyme included in the plurality of different partitions may be of a same type. Subsequent to co-partitioning, each biological particle of the plurality of biological particles within each partition of the plurality of different partitions of the plurality of separate partitions may be lysed or permeabilized, thereby providing access to RNA molecules of the plurality of RNA molecules of each biological particle. The RNA molecules may then be subjected to conditions suitable for each enzyme within each partition to digest RNA molecules of the second set of RNA molecules of each biological particle, thereby enriching the first set of RNA molecules of each biological particle within each partition. In this manner, a first set of RNA molecules (e.g., mRNA molecules) of a population of biological particles may be enriched simultaneously and without risk of contamination while retaining the RNA molecules in separate compartments. Subsequently, RNA molecules of the first set of RNA molecules of each biological particle within each partition of the plurality of different partitions of the plurality of separate partitions may be reverse transcribed using reverse transcriptase and nucleotide molecules co-partitioned with the biological particles. Reverse transcription of mRNA molecules within each partition may generate one or more cDNA molecules within each partition. These cDNA molecules may then be subjected to conditions suitable for performing one or more primer extension and/or amplification reactions (e.g., PCR) to generate amplification products in each partition. The amplification products may then be pooled to generate a pooled mixture. Nucleic acid sequences of at least a portion of the amplification products in the pooled mixture may then be detected using, for example, sequencing methods (e.g., as described herein). In some cases, each partition of the plurality of different partitions each including a single biological particle further comprise a bead comprising a plurality of nucleic acid barcode molecules attached thereto, where the plurality of nucleic acid barcode molecules comprise a common barcode sequence and a primer annealing sequence. The barcode sequence may be the same for each nucleic acid barcode molecule of the plurality of nucleic acid barcode molecules contained within a given partition, and the barcode sequence associated with each partition may be different. The plurality of nucleic acid barcode molecules may therefore be suitable for barcoding cDNA molecules. Amplification of the cDNA molecules within a given partition may comprise annealing a primer sequence of the nucleic acid barcode molecules within that partition to the cDNA molecules. The resultant amplification products of a given partition will then include a common barcode sequence that may be unique among a plurality of different partitions which may facilitate rapid and efficient sequencing of nucleic acid sequences originating from the plurality of biological particles. In some cases, the nucleic acid barcode molecules may additionally or alternatively be ligated to the cDNA molecules (e.g., using a ligase). In such cases, the ligation of the barcode molecule to the cDNA molecule may generate a barcoded nucleic acid molecule. In some instances, using a ligation approach may not be necessary to perform an amplification reaction to generate barcoded nucleic acid molecules. Alternatively, the plurality of nucleic acid barcode molecules coupled to a bead within a given partition may be suitable for barcoding RNA molecules. RNA molecules (e.g., RNA molecules of the first set of RNA molecules) may be barcoded and reverse transcribed simultaneously to provide barcoded cDNA molecules within the given partition. The barcoded cDNA molecules may then be released from the partition and undergo primer extension and/or amplification (e.g., in a pooled solution). Alternatively, the barcoded cDNA molecules may undergo primer extension and/or amplification within the given partition.

In an example, a method of processing a sample comprises providing a biological particle comprising a plurality of ribonucleic acid (RNA) molecules, wherein one or more RNA molecules of the plurality of RNA molecules is an mRNA molecule; co-partitioning the biological particle and one or more enrichment enzymes; lysing or permeabilizing the biological particle, thereby providing access to the plurality of RNA molecules of the biological particle; and bringing the plurality of RNA molecules in contact with an enrichment enzyme of the one or more enrichment enzymes to digest RNA molecules of the plurality of RNA molecules, wherein the RNA molecules of the plurality of RNA molecules digested by the enrichment enzyme are not mRNA molecules, thereby enriching the one or more mRNA molecules within the partition. The one or more enrichment enzymes may be exonucleases (e.g., as described herein). The biological particle and the one or more enrichment enzymes may be co-partitioned with one or more reagents, including, for example, reverse transcription enzymes and nucleotide molecules. The reverse transcription enzymes may be used to reverse transcribe the one or more mRNA molecules within the partition, thereby generating one or more cDNA molecules. The partition may further comprise a bead comprising a plurality of nucleic acid barcode molecules attached (e.g., releasably attached) thereto (e.g., as described herein). Each nucleic acid barcode molecule of the plurality of nucleic acid barcode molecules may comprise a common barcode sequence and one or more functional sequences, including, for example, a primer sequence. Following release of nucleic acid barcode molecules of the plurality of nucleic acid barcode molecules attached to the bead (e.g., upon application of a stimulus), the nucleic acid barcode molecules and the cDNA molecules may be used to synthesize barcoded nucleic acid products comprising sequences of the cDNA molecules and nucleic acid barcode molecules, or complements thereof. Synthesis of the barcoded nucleic acid products may comprise performing a primer extension reaction and/or nucleic acid amplification reaction (e.g., PCR). Such a reaction may comprise subjecting the partition to conditions sufficient to anneal primer sequences of the nucleic acid barcode molecules to the cDNA molecules to generate amplification products within the partition. The barcoded nucleic acid products may comprise a barcode sequence common to the plurality of nucleic acid barcode molecules attached (e.g., releasably attached) to the bead, or a complement thereof. The barcoded nucleic acid products may be recovered from the partition (e.g., by breaking an emulsion of droplets) and subjected to additional processing (e.g., amplification) and/or nucleic acid sequencing (e.g., as described herein). Alternatively, the plurality of nucleic acid barcode molecules may be used to barcode mRNA molecules within the partition. In some cases, barcoding may be achieved via a template switching process (e.g., using a nucleic acid barcode molecule that comprises a template switching oligonucleotide comprising a barcode sequence) to generate barcoded nucleic acid products. Where barcoding and reverse transcription are performed separately, barcoding may be performed within the partition while reverse transcription may be performed outside of the partition after recovery of barcoded mRNA molecules from the partition. In either case, amplification of barcoded mRNA or cDNA molecules may be performed outside of the partition. The methods provided herein may also be carried out for a plurality of biological particles in a plurality of different partitions (e.g., as described herein).

In another example, a method of processing a sample comprises providing a biological particle comprising a plurality of RNA molecules, wherein one or more RNA molecules of the plurality of RNA molecules is an mRNA molecule; co-partitioning the biological particle and one or more 5′-to-3′ exonucleases; lysing or permeabilizing the biological particle, thereby providing access to the plurality of RNA molecules of the biological particle; and bringing the plurality of RNA molecules in contact with a 5′-to-3′ exonuclease (e.g., the Terminator 5′-Phosphate-Dependent Exonuclease from Epicentre® Biotechnologies) to digest RNA molecules of the plurality of RNA molecules, wherein the RNA molecules of the plurality of RNA molecules digested by the 5′-to-3′ exonuclease are not mRNA molecules, thereby enriching the one or more mRNA molecules within the partition. The biological particle and the one or more 5′-to-3′ exonucleases may be co-partitioned with one or more reverse transcription enzymes and nucleotide molecules, as well as a bead comprising a plurality of nucleic acid barcode molecules attached thereto. Additional processing and analysis may be carried out as described in the preceding example. In some cases, the biological particle and the one or more 5′-to-3′ exonucleases may be co-partitioned with one or more reverse transcription enzymes and nucleotide molecules, polymerase enzymes, and a bead comprising a plurality of nucleic acid barcode molecules attached thereto. In some cases, the RNA molecules may be barcoded prior to undergoing prior to reverse transcription. In other cases, RNA molecules may undergo simultaneous barcoding and reverse transcription (e.g., as described herein).

In an example, a method of processing a sample comprises providing a cell comprising a plurality of RNA molecules, wherein one or more RNA molecules of the plurality of RNA molecules is an mRNA molecule; co-partitioning the cell and one or more 5′-to-3′ exonucleases within a droplet; lysing or permeabilizing the cell to provide access to the plurality of RNA molecules therein; and bringing the plurality of RNA molecules in contact with a 5′-to-3′ exonuclease to digest RNA molecules of the plurality of RNA molecules, wherein the RNA molecules of the plurality of RNA molecules digested by the 5′-to-3′ exonuclease are not mRNA molecules, thereby enriching the one or more mRNA molecules within the droplet. The biological particle and the one or more 5′-to-3′ exonucleases may be co-partitioned with one or more reverse transcription enzymes and nucleotide molecules, as well as a bead comprising a plurality of nucleic acid barcode molecules attached thereto. Additional processing and analysis may be carried out as described in the preceding examples.

In any of the preceding examples, the biological particle (e.g., cell, cell bead, or cell nucleus) may be co-partitioned with a gel bead comprising a plurality of nucleic acid barcode molecules releasably attached thereto. For example, the method may comprise providing a cell comprising a plurality of RNA molecules, wherein one or more RNA molecules of the plurality of RNA molecules is an mRNA molecule; co-partitioning the cell, a gel bead comprising a plurality of nucleic acid barcode molecules releasably attached thereto, reverse transcription enzymes, and one or more 5′-to-3′ exonucleases within a droplet; lysing or permeabilizing the cell to provide access to the plurality of RNA molecules therein; and bringing the plurality of RNA molecules in contact with a 5′-to-3′ exonuclease to digest RNA molecules of the plurality of RNA molecules, wherein the RNA molecules of the plurality of RNA molecules digested by the 5′-to-3′ exonuclease are not mRNA molecules, thereby enriching the one or more mRNA molecules within the droplet. The reverse transcription enzymes may then be used to reverse transcribe the one or more mRNA molecules within the droplet, thereby generating one or more cDNA molecules. A stimulus may then be applied to release nucleic acid barcode molecules from the bead. The nucleic acid barcode molecules may include primer sequences that may be annealed to sequences of the cDNA molecules and used to perform primer extension and/or amplification reactions (e.g., PCR) to generate barcoded nucleic acid products within the droplet. The nucleic acid barcode molecules may also include unique molecular identifiers and/or read primer sequences such as P5 and P7 primers that may be used in sequencing applications such as Illumina bridge amplification methods. The barcoded nucleic acid products may comprise a barcode sequence common to the plurality of nucleic acid barcode molecules releasably attached to the bead as well as a unique molecular identifier and/or read primer sequence, if used. The contents of the partition may then be recovered (e.g., by breaking an emulsion of droplets) and barcoded nucleic acid products may be sequenced (e.g., as described herein). In some cases, amplification may be performed after the contents of the partition are recovered. The method may also be carried out for a plurality of biological particles in a plurality of different partitions (e.g., as described herein).

In any of the preceding examples, the biological particle (e.g., cell, cell bead, or cell nucleus) may be co-partitioned with a gel bead comprising a plurality of nucleic acid barcode molecules releasably attached thereto. For example, the method may comprise providing a cell comprising a plurality of RNA molecules, wherein one or more RNA molecules of the plurality of RNA molecules is an mRNA molecule; co-partitioning the cell, a gel bead comprising a plurality of nucleic acid barcode molecules releasably attached thereto, reverse transcription enzymes, and one or more 5′-to-3′ exonucleases within a droplet; lysing or permeabilizing the cell to provide access to the plurality of RNA molecules therein; and bringing the plurality of RNA molecules in contact with a 5′-to-3′ exonuclease to digest RNA molecules of the plurality of RNA molecules, wherein the RNA molecules of the plurality of RNA molecules digested by the 5′-to-3′ exonuclease are not mRNA molecules, thereby enriching the one or more mRNA molecules within the droplet. A stimulus may then be applied to release nucleic acid barcode molecules of the plurality of nucleic acid barcode molecules from the bead. The nucleic acid barcode molecules may include primer sequences that may be configured to anneal to sequences of the one or more RNA molecules. Following annealing of nucleic acid barcode molecules to sequences of the one or more RNA molecules, the nucleic acid barcode molecules may be ligated to the one or more RNA molecules or to other molecules coupled thereto, e.g., using ligase to generate barcoded RNA molecules. For example, a nucleic acid barcode molecule may comprise a sequence complementary to a first sequence of an RNA molecule. A second molecule (e.g., a probe molecule) may comprise a sequence complementary to a second sequence of an RNA molecule. The nucleic acid barcode molecule may anneal to the first sequence of the RNA molecule and the second molecule may anneal to the second sequence of RNA molecule. The first and second sequences of the RNA molecule may be adjacent to one another. Alternatively, the first and second sequences may be separated by one or more nucleotides (such as between 1-500 nucleotides, such as between 10-100 nucleotides). The nucleic acid barcode molecule and the second molecule may be ligated to one another (e.g., using a chemical or enzymatic ligation process). An RNA molecule coupled to a nucleic acid barcode molecule may be considered a barcoded RNA molecule. Barcoded RNA molecules may be subjected to conditions sufficient to amplify and/or reverse transcribe the barcoded RNA molecules to generate barcoded cDNA molecules, which barcoded cDNA molecules each comprise one or more sequences complementary to one or more sequences of a barcoded RNA molecules. In some cases, the nucleic acid barcode molecules may also include unique molecular identifiers and/or read primer sequences such as P5 and P7 primers, or portions thereof that may be used in sequencing applications such as Illumina bridge amplification methods. Barcoded nucleic acid products synthesized using RNA molecules and nucleic acid barcode molecules may comprise a barcode sequence common to the plurality of nucleic acid barcode molecules releasably attached to the bead, or a complement thereof, as well as a unique molecular identifier and/or read primer sequence or complement thereof, if used. The contents of the partition may then be recovered and barcoded nucleic acid products may be subjected to further processing such as amplification and/or nucleic acid sequencing (e.g., as described herein). In some cases, a primer extension and/or amplification reaction may be performed after the contents of the partition are recovered. The method may also be carried out for a plurality of biological particles in a plurality of different partitions (e.g., as described herein).

In some cases, the nucleic acid molecules (e.g., mRNA or cDNA molecules) of or derived from a biological particle (e.g., cell, cell nucleus, or cell bead) may be barcoded combinatorially. The combinatorial barcoding scheme can be implemented using, e.g., a split-pool approach. For example, a plurality of permeabilized cells (or permeabilized nuclei or cell beads) may be partitioned into a first plurality of partitions (e.g., a plurality of wells) wherein each partition of the first plurality of partitions comprises a different (i.e., unique) first barcode sequence segment. Within each partition, the nucleic acid molecules may be barcoded using the first barcode sequence segment (e.g., via annealing, ligation and/or amplification, as described herein). After addition of the first barcode sequence segment, cells (or nuclei or cell beads) can be collected from the first plurality of partitions and pooled. The pooled cells (or nuclei or cell beads) may then be partitioned into a second plurality of partitions (e.g., a plurality of wells) wherein each partition of the second plurality of partitions comprises a different (i.e., unique) second barcode sequence segment. Repeating this split-pool process allows the generation of barcodes comprising any suitable number of barcode sequence segments. Combinatorial barcoding as described herein may comprise at least 1, 2, 3, 4, 5, 6, 7, 8 or more operations (e.g., split-pool cycles). Combinatorial barcoding comprising multiple operations may be useful, for example, in generation of greater barcode diversity and to synthesize a unique barcode sequence on nucleic acid molecules derived from each single cell (or cell nucleus or cell bead) of a plurality of cells (or cell nuclei or cell beads). For example, combinatorial barcoding comprising three operations, each comprising attachment of a unique nucleic acid sequence in each of 96 partitions, will yield up to 884,736 unique barcode combinations. Cells may be partitioned such that at least one cell (or nuclei or cell bead) is present in each partition of a plurality of partitions. Cells may be partitioned such that at least 1; 2; 3; 4; 5; 10; 20; 50; 100; 500; 1,000; 5,000; 10,000; 100,000; 1,000,000; or more cells are present in a single partition. Cells may be partitioned such that at most 1,000,000; 100,000; 10,000; 5,000; 1,000; 500; 100; 50; 20; 10; 5; 4; 3; 2; or 1 cell is present in a single partition. Cells may be partitioned in a random configuration.

In another example, the method may comprise providing a cell comprising a plurality of RNA molecules, wherein one or more RNA molecules of the plurality of RNA molecules is an mRNA molecule encoding at least a portion of a V(D)J sequence of an immune cell receptor, or a complement thereof. The method may comprise co-partitioning the cell, a gel bead comprising a plurality of nucleic acid barcode molecules releasably attached thereto, reverse transcription enzymes, and one or more 5′-to-3′ exonucleases within a droplet; lysing or permeabilizing the cell to provide access to the plurality of RNA molecules therein; and bringing the plurality of RNA molecules in contact with a 5′-to-3′ exonuclease to digest RNA molecules of the plurality of RNA molecules, wherein the RNA molecules of the plurality of RNA molecules digested by the 5′-to-3′ exonuclease are not mRNA molecules, thereby enriching the one or more mRNA molecules within the droplet. A stimulus may then be applied to release nucleic acid barcode molecules from the bead. The nucleic acid barcode molecules may include one or more features such as, for example, unique molecular identifiers, switch oligonucleotides, primer sequences, and read primer sequences such as P5 and P7 primers that may be used in sequencing applications such as Illumina bridge amplification methods. The nucleic acid barcode molecules may include a double-stranded region and/or a single-stranded region. In some cases, the nucleic acid barcode molecules are single-stranded. In some cases, the nucleic acid barcode molecules are double-stranded. In some cases, the nucleic acid barcode molecules comprise both a double-stranded and a single-stranded region. A nucleic acid barcode molecule may terminate in a sequence complementary to a poly(C) sequence, such as a poly(G) priming sequence. The poly(G) priming sequence may be a component of a template switching oligonucleotide that may be used in conjunction with a terminal transferase (or, for example, a reverse transcriptase with terminal transferase activity). An mRNA molecule comprising a poly(A) sequence (e.g., at its 3′ end) may be primed with a primer molecule comprising a poly(dT) sequence and a non-poly(dT) sequence. The primer may be extended (e.g., to the 5′ end of the mRNA molecule). The reverse transcriptase, which may have terminal transferase activity, may then add a poly(C) sequence at the 5′ end of the mRNA molecule and the molecule may then anneal to an end of a nucleic acid barcode molecule, priming the template switching oligonucleotide. Template switching may then occur and the transcript extension may be completed to include complements of the various components of the nucleic acid barcode molecule. In this manner, a cDNA molecule encoding at least a portion of a V(D)J sequence of an immune cell receptor may be generated from reverse transcription of a corresponding mRNA with a poly-T containing primer. The resultant cDNA molecule is barcoded with a barcode sequence and, in some cases, a UMI sequence. The original mRNA template and template switching oligonucleotide may be denatured from the cDNA and the barcoded primer comprising a sequence complementary to at least a portion of the generated priming region on the cDNA may then hybridize with the cDNA and a barcoded construct comprising the barcode sequence (and any optional UMI sequence) and a complement of the cDNA generated.

In another aspect, the present disclosure provides a partition comprising a biological particle (e.g., cell, cell bead, or cell nucleus) comprising a plurality of RNA molecules, wherein the plurality of RNA molecules comprises one or more mRNA molecules; and an enrichment enzyme that is configured to selectively degrade RNA molecules that are not mRNA molecules. The enrichment enzyme may be an exonuclease. The partition may be a droplet (e.g., an aqueous droplet) or a well. In some cases, the partition may further comprise one or more reagents selected from the group consisting of fluorophores, oligonucleotides, primers, nucleic acid barcode molecules, barcodes, buffers, deoxynucleotide triphosphates, detergents, reducing agents, chelating agents, oxidizing agents, nanoparticles, antibodies, temperature-sensitive enzymes, pH-sensitive enzymes, light-sensitive enzymes, proteases, ligases, polymerases, reverse transcription enzymes, restriction enzymes, transposase enzymes, nucleases, protease inhibitors, and nuclease inhibitors. The partition may also comprise a bead (e.g., a gel bead) comprising a plurality of nucleic acid barcode molecules attached thereto. The plurality of nucleic acid barcode molecules may be releasably attached to the bead, and may be released from the bead upon application of a stimulus (e.g., as described herein).

In another aspect, the present disclosure provides a method for sample processing comprising providing a sample comprising a nucleic acid molecule (e.g., a messenger ribonucleic acid (mRNA) molecule). The nucleic acid molecule of the sample may be included within a biological particle (e.g., cell, cell bead, or cell nucleus), which biological particle may comprise a plurality of nucleic acid molecules, including one or more nucleic acid molecules that are digestible by an enzyme such as an exonuclease. The nucleic acid molecule may be provided within a partition. The nucleic acid molecule (e.g., mRNA molecule) may have a first target region and a second target region. A portion of a first probe may be hybridized to a portion of the first target region and a portion of a second probe may be hybridized to a portion of the second target region. In some cases, this hybridization process may take place within a partition (e.g., droplet or well). In other cases, the hybridization may take place outside of a partition. The first and second probes may hybridize to the nucleic acid molecule at the same or different times during sample processing. For example, the first probe may hybridize to the nucleic acid molecule outside of a partition, and the second probe may hybridize to the nucleic acid molecule inside a partition (e.g., subsequent to a partitioning process). In some cases, the nucleic acid molecule and the first probe may be subjected to conditions sufficient to hybridize the first probe to the first target region of the nucleic acid molecule. The nucleic acid molecule may be included within a biological particle within a partition, where the biological particle may comprise a plurality of nucleic acid molecules and where the partition comprises the second probe. The nucleic acid molecule and the second probe may be subjected to conditions sufficient to hybridize the second probe to the second target region of the nucleic acid molecule. Nucleic acid molecules of the plurality of nucleic acid molecules may be digested (e.g., using an exonuclease, as described herein) to enrich the nucleic acid molecule within the plurality of nucleic acid molecules. Digestion may take place before hybridization of the first probe to the nucleic acid molecule, between hybridization of the first and second probes to the nucleic acid molecule, or after hybridization of the first and second probes to the nucleic acid molecule. Digestion may take place within or outside of the partition.

The first target region of the nucleic acid molecule may be adjacent to the second target region of the nucleic acid molecule. Alternatively, the first target region may be separated from the second target region by one or more nucleotides, such as between 1-500 nucleotides, such as between 50-100 nucleotides.

The portion of the first probe may be a nucleic acid sequence, and the portion of the second probe may also be a nucleic acid sequence. Each of the probes may comprise sequences that are capable of binding to adapter molecules (e.g., adapter molecules having the same or different sequences). The portion of the first probe may also comprise a first reactive moiety, and the portion of the second probe may comprise a second reactive moiety. When the first and second probes are hybridized to the first and second target regions of the nucleic acid molecule, the first and second reactive moieties may be adjacent to one another. A reactive moiety of a probe may be selected from the non-limiting group consisting of azides, alkynes, nitrones (e.g., 1,3-nitrones), strained alkenes (e.g., trans-cycloalkenes such as cyclooctenes or oxanorbornadiene), tetrazines, tetrazoles, iodides, thioates (e.g., phorphorothioate), acids, amines, and phosphates. For example, the first reactive moiety of a first probe may comprise an azide moiety, and a second reactive moiety of a second probe may comprise an alkyne moiety. The reactive moieties may be subjected to conditions sufficient to cause them to react to yield a probe-linked nucleic acid molecule comprising the first probe linked to the second probe. The first and second reactive moieties may react to form a linking moiety. A reaction between the first and second reactive moieties may be, for example, a cycloaddition reaction such as a strain-promoted azide-alkyne cycloaddition, a copper-catalyzed azide-alkyne cycloaddition, a strain-promoted alkyne-nitrone cycloaddition, a Diels-Alder reaction, a [3+2] cycloaddition, a [4+2] cycloaddition, or a [4+1] cycloaddition; a thiol-ene reaction; a nucleophilic substation reaction; or another reaction. In some cases, reaction between the first and second reactive moieties may yield a triazole moiety or an isoxazoline moiety. A reaction between the first and second reactive moieties may involve subjecting the reactive moieties to suitable conditions such as a suitable temperature, pH, or pressure and providing one or more reagents or catalysts for the reaction. For example, a reaction between the first and second reactive moieties may be catalyzed by a copper catalyst, a ruthenium catalyst, or a strained species such as a difluorooctyne, dibenzylcyclooctyne, or biarylazacyclooctynone. Reaction between a first reactive moiety of a first probe sequence of a first probe hybridized to a first target region of the nucleic acid molecule and a second reactive moiety of a third probe sequence of a second probe hybridized to a second target region of the nucleic acid molecule may link the first probe and the second probe to provide a probe-linked nucleic acid molecule. Upon linking, the first and second probes may be considered ligated. Accordingly, reaction of the first and second reactive moieties may comprise a chemical ligation reaction such as a copper-catalyzed 5′ azide to 3′ alkyne “click” chemistry reaction to form a triazole linkage between two probes. In other non-limiting examples, an iodide moiety may be chemically ligated to a phosphorothioate moiety to form a phosphorothioate bond, an acid may be ligated to an amine to form an amide bond, and/or a phosphate and amine may be ligated to form a phosphoramidate bond.

In some cases, the first and second probes do not comprise reactive moieties. Such first and second probes may be subjected to a nucleic acid reaction to provide a probe-linked nucleic acid molecule. For example, the first and second probes may be subjected to an enzymatic ligation reaction, e.g., using a ligase (e.g., SplintR ligase and/or T4 or T4 ligase). Following the enzymatic ligation reaction, the first and second probes may be considered ligated. Where the first and second target regions are not adjacent to one another, the probes and/or the nucleic acid molecule may be subjected to conditions sufficient to link the first probe to the second probe (e.g., “fill in” the gap or space disposed between the first and the second probes). The probes may be subjected to an enzymatic ligation reaction, using a ligase, e.g., SplintR ligases, T4 ligases, PBCV1 enzymes. Gaps between the first and second probes hybridized to the nucleic acid molecule may first be filled prior to ligation, using, for example, Mu polymerase, DNA polymerase, RNA polymerase, reverse transcriptase, VENT polymerase, Taq polymerase, and/or any combinations, derivatives, and variants (e.g., engineered mutants) thereof. In some embodiments, ribonucleotides are ligated between the first and second probes. In some embodiments, deoxyribonucleotides are ligated between the first and second probes.

The first and second probes may comprise any number and combination of useful sequences. For example, the first and/or second probe may comprise an adapter sequence, a barcode sequence, a UMI sequence, a sequencing primer sequence (e.g., a P5 or P7 primer sequence) or portion thereof, a restriction site, a spacer sequence, a transposition site, etc. In some cases, the first probe or the second probe may be attached to a bead (e.g., as described herein). In some cases, both the first probe and the second probe are attached to a bead.

In some cases, the first and second probes may be both present in a linear nucleic acid molecule. The linear nucleic acid molecule may be a molecular inversion probe. In some cases, one or more probes may comprise a padlock probe or a molecular inversion probe.

The nucleic acid molecule having the first and/or second probe hybridized thereto (in some cases, the probe-linked nucleic acid molecule) may be barcoded (e.g., within a partition) to provide a barcoded nucleic acid molecule (e.g., barcoded probe-linked nucleic acid molecule). Barcoding may comprise hybridizing a binding sequence of a nucleic acid barcode molecule to a portion of the first probe hybridized to the nucleic acid molecule. The nucleic acid barcode molecule may be attached (e.g., releasably attached) to a bead (e.g., as described herein). The first probe or nucleic acid barcode molecule may subsequently be subjected to a primer extension reaction. For example, the first probe may be extended from an end of the first probe to an end of the nucleic acid barcode molecule to which it is hybridized to provide an extended nucleic acid molecule. The extended nucleic acid barcode molecule may comprise the first probe, the second probe, a sequence complementary to the barcode sequence of the nucleic acid barcode molecule, and a sequence complementary to another sequence (e.g., another binding sequence) of the nucleic acid barcode molecule. The extended nucleic acid molecule may be denatured from the nucleic acid barcode molecule and the nucleic acid molecule of interest and then duplicated or amplified (e.g., using polymerase chain reactions (PCR) or linear amplification) to facilitate detection of the extended nucleic acid molecule or a complement thereof (e.g., an amplified product) by, e.g., sequencing. One or more of the methods described herein may allow for genomic, transcriptomic, or exomic profiling. One or more of the methods described herein may allow for profiling of non-polyadenylated targets (e.g., non-poly-A RNAs), splice junctions, single nucleotide polymorphisms (SNPs), fixed cells, etc.

The methods described herein may facilitate gene expression profiling with single cell resolution using, for example, chemical ligation-mediated barcoding, amplification, and sequencing. The methods described herein may allow for gene expression analysis while avoiding the use of enzymatic ligation, specialized imaging equipment, and reverse transcription, which may be highly error prone and inefficient. For example, the methods may be used to analyze a pre-determined panel of target genes in a population of single cells in a sensitive and accurate manner. In some cases, the nucleic acid molecule analyzed by the methods described herein may be a fusion gene (e.g., a hybrid gene generated via translocation, interstitial deletion, or chromosomal inversion).

Any combination or variations of the processes described herein may be performed in any convenient step of the methods described herein. In one non-limiting example, the method may comprise providing a cell comprising a plurality of RNA molecules, wherein one or more RNA molecules of the plurality of RNA molecules is an mRNA molecule; co-partitioning the cell, a gel bead comprising a plurality of first and/or second probes releasably attached thereto, reverse transcription enzymes, and one or more 5′-to-3′ exonucleases within a droplet; lysing or permeabilizing the cell to provide access to the plurality of RNA molecules therein; and bringing the plurality of RNA molecules in contact with a 5′-to-3′ exonuclease to digest RNA molecules of the plurality of RNA molecules, wherein the RNA molecules of the plurality of RNA molecules digested by the 5′-to-3′ exonuclease are not mRNA molecules, thereby enriching the one or more mRNA molecules within the droplet. The reverse transcription enzymes may then be used to reverse transcribe the one or more mRNA molecules within the droplet, thereby generating one or more cDNA molecules. A stimulus may then be applied to release the plurality of first and/or second probe molecules from the bead. The first probes and/or the second probes may comprise a barcode sequence. Additionally or alternatively, the first probes and/or the second probes may include primer sequences that may be annealed to cDNA molecules. First and second probes may be hybridized to first and second target regions of the cDNA molecules. The first and second probe hybridized to a given cDNA molecule may be linked (e.g., enzymatically or via “click” chemistry approaches, as described herein). In some cases, the cDNA molecule may be barcoded via hybridization of a first and/or second probe comprising a barcode sequence. In other cases, the cDNA molecule may be barcoded via coupling of a nucleic acid barcode molecule to a first or second probe hybridized to the cDNA molecule (e.g., via hybridization or ligation, and/or via a splint molecule, as described herein). In some cases, an mRNA molecule may be barcoded (e.g., as described herein), such that reverse transcription of the barcoded mRNA molecule provides a barcoded cDNA molecule. In some cases, a barcoded nucleic acid product may be generated during via a template switching process (e.g., using a barcoded template switch oligonucleotide, as described herein). Additional barcoding processes are described herein. In addition to a barcode sequence, a barcoding process may add one or more additional sequences to a cDNA molecule or mRNA molecule. For example, a barcoding process may add a UMI sequence and/or a read primer sequences such as P5 and P7 primers that may be used in sequencing applications such as Illumina bridge amplification methods. Such sequences may be components of a nucleic acid barcode molecule (e.g., as described herein).

A primer molecule (e.g., a nucleic acid barcode molecule) and the cDNA molecule may then be used to synthesize one or more barcoded nucleic acid products (e.g., via a primer extension and/or nucleic acid amplification reaction). The one or more barcoded nucleic acid products may be synthesized within the partition (e.g., droplet) or outside of the droplet. For example, a barcoded cDNA molecule may be generated within the partition and subsequently recovered from the partition, and then the barcoded cDNA molecule may be used to synthesize one or more barcoded nucleic acid products (e.g., in a pooled solution). The barcoded nucleic acid products may comprise a barcode sequence common to the plurality of nucleic acid barcode molecules releasably attached to the bead as well as a unique molecular identifier and/or read primer sequence, if used. Barcoded nucleic acid products may undergo nucleic acid sequencing (e.g., as described herein). The method may also be carried out for a plurality of biological particles in a plurality of different partitions (e.g., as described herein).

In another example, the method may comprise providing a cell comprising a plurality of RNA molecules, wherein one or more RNA molecules of the plurality of RNA molecules is an mRNA molecule; co-partitioning the cell, a bead (e.g., gel bead) comprising a plurality of nucleic acid barcode molecules releasably attached thereto, one or more 5′-to-3′ exonucleases within a droplet, and, optionally, first and second probes; lysing or permeabilizing the cell to provide access to the plurality of RNA molecules therein; bringing the plurality of RNA molecules in contact with a 5′-to-3′ exonuclease to digest RNA molecules of the plurality of RNA molecules, wherein the RNA molecules of the plurality of RNA molecules digested by the 5′-to-3′ exonuclease are not mRNA molecules, thereby enriching the one or more mRNA molecules within the droplet; and hybridizing the first and the second probes to the mRNA molecules. The first and second probes may not comprise barcode sequences. The plurality of nucleic acid barcode molecules may each comprise a common barcode sequence as well as one or more additional functional sequences, such as a UMI sequence, a reader primer sequence (e.g., P5 or P7 sequence) or portion thereof, an overhang sequence, and a sequencing primer or portion thereof. The plurality of nucleic acid barcode molecules may comprise a first set of nucleic acid barcode molecules and a second set of nucleic acid barcode molecules, where the first set of nucleic acid barcode molecules and the second set of nucleic acid barcode molecules comprise one or more different functional sequences (e.g., as described herein). Nucleic acid barcode molecules of the plurality of nucleic acid barcode molecules releasably attached to the bead may be released from the bead (e.g., upon application of a stimulus, as described herein). Nucleic acid barcode molecules may hybridize or ligate to first probe hybridized to mRNA molecules of the one or more mRNA molecules. The first probes hybridized to the mRNA molecules may be linked to the second probes hybridized to the mRNA molecules (e.g., before or after coupling of nucleic acid barcode molecules to the first probes (e.g., via enzymatic or chemical ligation, as described herein). The mRNA molecules may be reverse transcribed (e.g., using reverse transcription enzymes) to provide cDNA molecules within the partition. Reverse transcription may take place before or after barcoding of the mRNA molecules. For example, mRNA molecules may be barcoded as described above and may then undergo reverse transcription to provide barcoded cDNA molecules, which barcoded cDNA molecules or derivatives thereof may be used to synthesize barcoded nucleic acid products (e.g., via primer extension and/or amplification reactions carried out within or outside of the partition). The barcoded nucleic acid products may comprise the common barcode sequence of the nucleic acid barcode molecules or a complement thereof. Alternatively, first and second probes may not be used, and reverse transcription may be performed using reverse transcriptase enzymes with terminal transferase activity may append a sequence such as a poly(C) sequence to an end of each of the cDNA molecules, and template switching oligonucleotides comprising a sequence complementary to the appended sequence (e.g., a poly(G) sequence) and a common barcode sequence may be used to provide barcoded nucleic acid products comprising the common barcode sequence or a complement thereof. The barcoded nucleic acid products may then undergo amplification (e.g., within or outside of the partition) and sequencing (e.g., as described herein). The template switching oligonucleotides may be the nucleic acid barcode molecules releasably attached to the bead or other nucleic acid molecules. The method may also be carried out for a plurality of biological particles in a plurality of different partitions (e.g., as described herein).

One or more processes described herein may occur inside a partition (e.g., well or droplet) or outside a partition (e.g., in bulk or in a bulk container). One or more processes may also occur in any convenient or useful order and may be repeated any number of times. For example, barcoding may occur using the first set of molecules (e.g., mRNA molecules) prior to reverse transcription. In examples where multiple probes (e.g., first and second probes) are used, a first probe may be hybridized to the target nucleic acid molecule (e.g., mRNA molecule) or a subset of the first set of molecules (e.g., mRNA molecules). The first probe may then be barcoded, e.g., using an adapter molecule and a barcode molecule, a splinted barcode molecule, or any combination or derivatives thereof (e.g., as described herein). The barcode molecule and the probe may be ligated (e.g., using click chemistry or enzymatically). In some cases, unhybridized probes may then be digested (e.g., using an exonuclease). Subsequently, a second probe molecule may be introduced, which may hybridize to the target nucleic acid molecule, adjacent to the barcoded probe molecule. The second probe molecule may then be ligated (e.g. using click chemistry or enzymatically) to the first probe to form a barcoded probe-linked nucleic acid molecule. In some cases, the barcoding may occur prior to, during, or following partitioning. Similarly, ligation and/or digestion (e.g., as described herein) may occur in a partition or outside of a partition.

The methods described herein may be applied to any nucleic acid molecule of interest. For example, the present disclosure may provide a method for use in processing or analyzing a sample, comprising: providing a biological particle (e.g., a cell, cell nucleus, or cell bead) comprising a plurality of molecules (e.g., a plurality of deoxyribonucleic acid [DNA] molecules), wherein the plurality of molecules comprises a first set of molecules (e.g., a first set of DNA molecules) and a second set of molecules (e.g., a second set of DNA molecules, a set of RNA molecules, etc.); co-partitioning the biological particle and an enzyme in a partition (e.g., a droplet or well); lysing or permeabilizing the biological particle, thereby providing access to the plurality of molecules of the biological particle; and subjecting the plurality of molecules within the partition to conditions suitable for the enzyme to digest molecules of the second set of molecules, thereby increasing a concentration or amount of the first set of molecules relative to the second set of molecules within the partition.

A method of enriching and processing RNA molecules (e.g., mRNA molecules) within a plurality of molecules (e.g., within a partition) may be performed in tandem with a method of processing other nucleic acid molecules. For example, in any of the methods described herein, a biological particle (e.g., cell, cell bead, or cell nucleus) may be provided within a partition (e.g., droplet or well), wherein the biological particle comprises a plurality of molecules. The plurality of molecules may comprise a plurality of RNA molecules and a plurality of DNA molecules. The plurality of RNA molecules may comprise a first set of RNA molecules (e.g., mRNA molecules) and a second set of RNA molecules, wherein the second set of RNA molecules may not comprise an mRNA molecule. The first set of RNA molecules (e.g., mRNA molecules) may be enriched within the plurality of molecules within the partition via digestion of at least a subset of the second set of RNA molecules (e.g., as described herein). The first set of RNA molecules (e.g., mRNA molecules) may then be subjected to processing (e.g., barcoding, reverse transcription, amplification, sequencing, etc., as described herein). All or a subset of the plurality of DNA molecules may be processed in tandem. For example, all or a subset of the plurality of DNA molecules may be subjected to a tagmentation process (e.g., prior to providing the biological particle in the partition) to provide tagmented fragments, which tagmented fragments correspond to regions of accessible chromatin. The tagmented fragments may then undergo processing including barcoding within the partition and may undergo amplification (e.g., within or outside of the partition) and sequencing. In some cases, the same nucleic acid barcode molecules that are used in the processing of RNA molecules (e.g., mRNA molecules) may be used in the processing of the tagmented fragments. In other cases, a first set of nucleic acid barcode molecules may be used to process RNA molecules and a second set of nucleic acid barcode molecules may be used to process tagmented fragments. The first and second sets of nucleic acid barcode molecules may be coupled to the same bead (e.g., as described herein).

### Systems and Methods for Sample Compartmentalization

In an aspect, the systems and methods described herein provide for the compartmentalization, depositing, or partitioning of one or more particles (e.g., biological particles, macromolecular constituents of biological particles, beads, reagents, etc.) into discrete compartments or partitions (referred to interchangeably herein as partitions), where each partition maintains separation of its own contents from the contents of other partitions. The partition can be a droplet in an emulsion. A partition may comprise one or more other partitions.

A partition may include one or more particles. A partition may include one or more types of particles. For example, a partition of the present disclosure may comprise one or more biological particles and/or macromolecular constituents thereof. A partition may comprise one or more gel beads. A partition may comprise one or more cell beads. A partition may include a single gel bead, a single cell bead, or both a single cell bead and single gel bead. A partition may include one or more reagents. Alternatively, a partition may be unoccupied. For example, a partition may not comprise a bead. A cell bead can be a biological particle and/or one or more of its macromolecular constituents encased inside of a gel or polymer matrix, such as via polymerization of a droplet containing the biological particle and precursors capable of being polymerized or gelled. Unique identifiers, such as barcodes, may be injected into the droplets previous to, subsequent to, or concurrently with droplet generation, such as via a microcapsule (e.g., bead), as described elsewhere herein. Microfluidic channel networks (e.g., on a chip) can be utilized to generate partitions as described herein. Alternative mechanisms may also be employed in the partitioning of individual biological particles, including porous membranes through which aqueous mixtures of cells are extruded into non-aqueous fluids.

The partitions can be flowable within fluid streams. The partitions may comprise, for example, micro-vesicles that have an outer barrier surrounding an inner fluid center or core. In some cases, the partitions may comprise a porous matrix that is capable of entraining and/or retaining materials within its matrix. The partitions can be droplets of a first phase within a second phase, wherein the first and second phases are immiscible. For example, the partitions can be droplets of aqueous fluid within a non-aqueous continuous phase (e.g., oil phase). In another example, the partitions can be droplets of a non-aqueous fluid within an aqueous phase. In some examples, the partitions may be provided in a water-in-oil emulsion or oil-in-water emulsion. A variety of different vessels are described in, for example, U.S. Patent Application Publication No. 2014/0155295, which is entirely incorporated herein by reference for all purposes. Emulsion systems for creating stable droplets in non-aqueous or oil continuous phases are described in, for example, U.S. Patent Application Publication No. 2010/0105112, which is entirely incorporated herein by reference for all purposes.

In the case of droplets in an emulsion, allocating individual particles to discrete partitions may in one non-limiting example be accomplished by introducing a flowing stream of particles in an aqueous fluid into a flowing stream of a non-aqueous fluid, such that droplets are generated at the junction of the two streams. Fluid properties (e.g., fluid flow rates, fluid viscosities, etc.), particle properties (e.g., volume fraction, particle size, particle concentration, etc.), microfluidic architectures (e.g., channel geometry, etc.), and other parameters may be adjusted to control the occupancy of the resulting partitions (e.g., number of biological particles per partition, number of beads per partition, etc.). For example, partition occupancy can be controlled by providing the aqueous stream at a certain concentration and/or flow rate of particles. To generate single biological particle partitions, the relative flow rates of the immiscible fluids can be selected such that, on average, the partitions may contain less than one biological particle per partition in order to ensure that those partitions that are occupied are primarily singly occupied. In some cases, partitions among a plurality of partitions may contain at most one biological particle (e.g., bead, DNA, cell or cellular material). In some embodiments, the various parameters (e.g., fluid properties, particle properties, microfluidic architectures, etc.) may be selected or adjusted such that a majority of partitions are occupied, for example, allowing for only a small percentage of unoccupied partitions. The flows and channel architectures can be controlled as to ensure a given number of singly occupied partitions, less than a certain level of unoccupied partitions and/or less than a certain level of multiply occupied partitions.

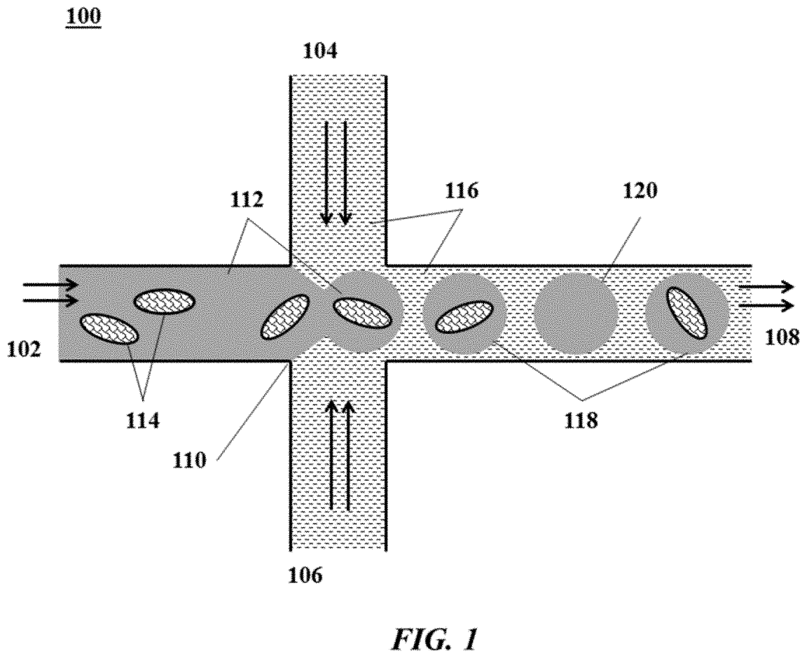

FIG. 1 shows an example of a microfluidic channel structure 100 for partitioning individual biological particles. The channel structure 100 can include channel segments 102, 104, 106 and 108 communicating at a channel junction 110. In operation, a first aqueous fluid 112 that includes suspended biological particles (or cells) 114 may be transported along channel segment 102 into junction 110, while a second fluid 116 that is immiscible with the aqueous fluid 112 is delivered to the junction 110 from each of channel segments 104 and 106 to create discrete droplets 118, 120 of the first aqueous fluid 112 flowing into channel segment 108, and flowing away from junction 110. The channel segment 108 may be fluidically coupled to an outlet reservoir where the discrete droplets can be stored and/or harvested. A discrete droplet generated may include an individual biological particle 114 (such as droplets 118). A discrete droplet generated may include more than one individual biological particle 114 (not shown in FIG. 1). A discrete droplet may contain no biological particle 114 (such as droplet 120). Each discrete partition may maintain separation of its own contents (e.g., individual biological particle 114) from the contents of other partitions.

The second fluid 116 can comprise an oil, such as a fluorinated oil, that includes a fluorosurfactant for stabilizing the resulting droplets, for example, inhibiting subsequent coalescence of the resulting droplets 118, 120. Examples of particularly useful partitioning fluids and fluorosurfactants are described, for example, in U.S. Patent Application Publication No. 2010/0105112, which is entirely incorporated herein by reference for all purposes.

As will be appreciated, the channel segments described herein may be coupled to any of a variety of different fluid sources or receiving components, including reservoirs, tubing, manifolds, or fluidic components of other systems. As will be appreciated, the microfluidic channel structure 100 may have other geometries. For example, a microfluidic channel structure can have more than one channel junction. For example, a microfluidic channel structure can have 2, 3, 4, or 5 channel segments each carrying particles (e.g., biological particles, cell beads, and/or gel beads) that meet at a channel junction. Fluid may be directed to flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electrokinetic pumping, vacuum, capillary or gravity flow, or the like.

The generated droplets may comprise two subsets of droplets: (1) occupied droplets 118, containing one or more biological particles 114, and (2) unoccupied droplets 120, not containing any biological particles 114. Occupied droplets 118 may comprise singly occupied droplets (having one biological particle) and multiply occupied droplets (having more than one biological particle). As described elsewhere herein, in some cases, the majority of occupied partitions can include no more than one biological particle per occupied partition and some of the generated partitions can be unoccupied (of any biological particle). In some cases, though, some of the occupied partitions may include more than one biological particle. In some cases, the partitioning process may be controlled such that fewer than about 25% of the occupied partitions contain more than one biological particle, and in many cases, fewer than about 20% of the occupied partitions have more than one biological particle, while in some cases, fewer than about 10% or even fewer than about 5% of the occupied partitions include more than one biological particle per partition.

In some cases, it may be desirable to minimize the creation of excessive numbers of empty partitions, such as to reduce costs and/or increase efficiency. While this minimization may be achieved by providing a sufficient number of biological particles (e.g., biological particles 114) at the partitioning junction 110, such as to ensure that at least one biological particle is encapsulated in a partition, the Poissonian distribution may expectedly increase the number of partitions that include multiple biological particles. As such, where singly occupied partitions are to be obtained, at most about 95%, 90%, 85%, 80%, 75%, 70%, 65%, 60%, 55%, 50%, 45%, 40%, 35%, 30%, 25%, 20%, 15%, 10%, 5% or less of the generated partitions can be unoccupied.

In some cases, the flow of one or more of the biological particles (e.g., in channel segment 102), or other fluids directed into the partitioning junction (e.g., in channel segments 104, 106) can be controlled such that, in many cases, no more than about 50% of the generated partitions, no more than about 25% of the generated partitions, or no more than about 10% of the generated partitions are unoccupied. These flows can be controlled so as to present a non-Poissonian distribution of single-occupied partitions while providing lower levels of unoccupied partitions. The above noted ranges of unoccupied partitions can be achieved while still providing any of the single occupancy rates described above. For example, in many cases, the use of the systems and methods described herein can create resulting partitions that have multiple occupancy rates of less than about 25%, less than about 20%, less than about 15%, less than about 10%, and in many cases, less than about 5%, while having unoccupied partitions of less than about 50%, less than about 40%, less than about 30%, less than about 20%, less than about 10%, less than about 5%, or less.

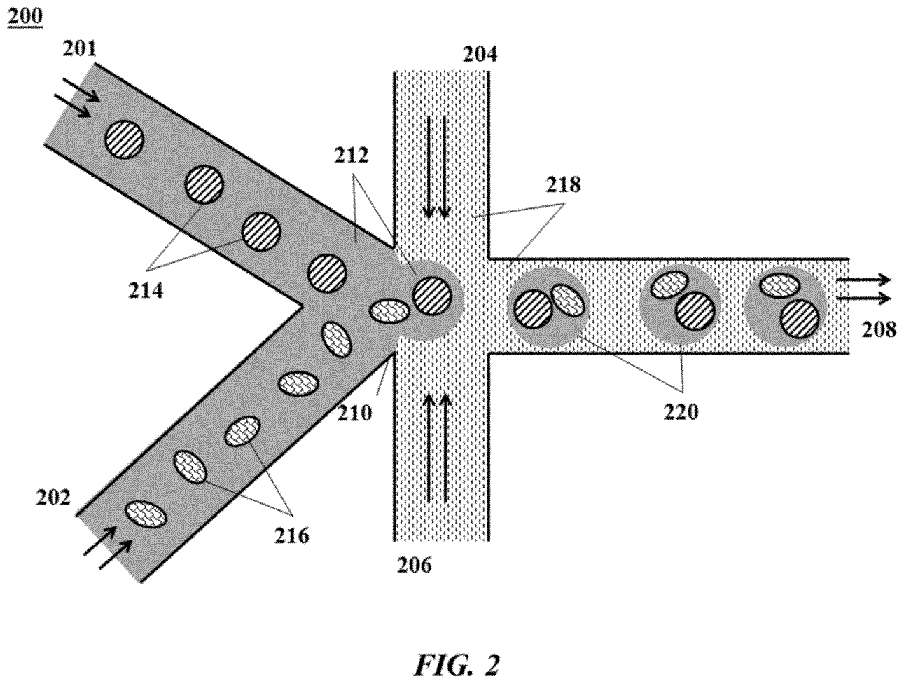

As will be appreciated, the above-described occupancy rates are also applicable to partitions that include both biological particles and additional reagents, including, but not limited to, microcapsules or beads (e.g., gel beads) carrying barcoded nucleic acid molecules (e.g., oligonucleotides) (described in relation to FIG. 2). The occupied partitions (e.g., at least about 10%, 20%, 30%, 40%, 50%, 60%, 70%, 80%, 90%, 95%, or 99% of the occupied partitions) can include both a microcapsule (e.g., bead) comprising barcoded nucleic acid molecules and a biological particle.

In another aspect, in addition to or as an alternative to droplet based partitioning, biological particles may be encapsulated within a microcapsule that comprises an outer shell, layer or porous matrix in which is entrained one or more individual biological particles or small groups of biological particles. The microcapsule may include other reagents. Encapsulation of biological particles may be performed by a variety of processes. Such processes may combine an aqueous fluid containing the biological particles with a polymeric precursor material that may be capable of being formed into a gel or other solid or semi-solid matrix upon application of a particular stimulus to the polymer precursor. Such stimuli can include, for example, thermal stimuli (e.g., either heating or cooling), photo-stimuli (e.g., through photo-curing), chemical stimuli (e.g., through crosslinking, polymerization initiation of the precursor (e.g., through added initiators)), mechanical stimuli, or a combination thereof.

Preparation of microcapsules comprising biological particles may be performed by a variety of methods. For example, air knife droplet or aerosol generators may be used to dispense droplets of precursor fluids into gelling solutions in order to form microcapsules that include individual biological particles or small groups of biological particles. Likewise, membrane based encapsulation systems may be used to generate microcapsules comprising encapsulated biological particles as described herein. Microfluidic systems of the present disclosure, such as that shown in FIG. 1, may be readily used in encapsulating cells as described herein. In particular, and with reference to FIG. 1, the aqueous fluid 112 comprising (i) the biological particles 114 and (ii) the polymer precursor material (not shown) is flowed into channel junction 110, where it is partitioned into droplets 118, 120 through the flow of non-aqueous fluid 116. In the case of encapsulation methods, non-aqueous fluid 116 may also include an initiator (not shown) to cause polymerization and/or crosslinking of the polymer precursor to form the microcapsule that includes the entrained biological particles. Examples of polymer precursor/initiator pairs include those described in U.S. Patent Application Publication No. 2014/0378345, which is entirely incorporated herein by reference for all purposes.

For example, in the case where the polymer precursor material comprises a linear polymer material, such as a linear polyacrylamide, PEG, or other linear polymeric material, the activation agent may comprise a cross-linking agent, or a chemical that activates a cross-linking agent within the formed droplets. Likewise, for polymer precursors that comprise polymerizable monomers, the activation agent may comprise a polymerization initiator. For example, in certain cases, where the polymer precursor comprises a mixture of acrylamide monomer with a N,N′-bis-(acryloyl)cystamine (BAC) comonomer, an agent such as tetraethylmethylenediamine (TEMED) may be provided within the second fluid streams 116 in channel segments 104 and 106, which can initiate the copolymerization of the acrylamide and BAC into a cross-linked polymer network, or hydrogel.

Upon contact of the second fluid stream 116 with the first fluid stream 112 at junction 110, during formation of droplets, the TEMED may diffuse from the second fluid 116 into the aqueous fluid 112 comprising the linear polyacrylamide, which will activate the crosslinking of the polyacrylamide within the droplets 118, 120, resulting in the formation of gel (e.g., hydrogel) microcapsules, as solid or semi-solid beads or particles entraining the cells 114. Although described in terms of polyacrylamide encapsulation, other ‘activatable’ encapsulation compositions may also be employed in the context of the methods and compositions described herein. For example, formation of alginate droplets followed by exposure to divalent metal ions (e.g., Ca' ions), can be used as an encapsulation process using the described processes. Likewise, agarose droplets may also be transformed into capsules through temperature based gelling (e.g., upon cooling, etc.).

In some cases, encapsulated biological particles can be selectively releasable from the microcapsule, such as through passage of time or upon application of a particular stimulus, that degrades the microcapsule sufficiently to allow the biological particles (e.g., cell), or its other contents to be released from the microcapsule, such as into a partition (e.g., droplet). For example, in the case of the polyacrylamide polymer described above, degradation of the microcapsule may be accomplished through the introduction of an appropriate reducing agent, such as DTT or the like, to cleave disulfide bonds that cross-link the polymer matrix. See, for example, U.S. Patent Application Publication No. 2014/0378345, which is entirely incorporated herein by reference for all purposes.

The biological particle can be subjected to other conditions sufficient to polymerize or gel the precursors. The conditions sufficient to polymerize or gel the precursors may comprise exposure to heating, cooling, electromagnetic radiation, and/or light. The conditions sufficient to polymerize or gel the precursors may comprise any conditions sufficient to polymerize or gel the precursors. Following polymerization or gelling, a polymer or gel may be formed around the biological particle. The polymer or gel may be diffusively permeable to chemical or biochemical reagents. The polymer or gel may be diffusively impermeable to macromolecular constituents of the biological particle. In this manner, the polymer or gel may act to allow the biological particle to be subjected to chemical or biochemical operations while spatially confining the macromolecular constituents to a region of the droplet defined by the polymer or gel. The polymer or gel may include one or more of disulfide cross-linked polyacrylamide, agarose, alginate, polyvinyl alcohol, polyethylene glycol (PEG)-diacrylate, PEG-acrylate, PEG-thiol, PEG-azide, PEG-alkyne, other acrylates, chitosan, hyaluronic acid, collagen, fibrin, gelatin, or elastin. The polymer or gel may comprise any other polymer or gel.

The polymer or gel may be functionalized to bind to targeted analytes, such as nucleic acids, proteins, carbohydrates, lipids or other analytes. The polymer or gel may be polymerized or gelled via a passive mechanism. The polymer or gel may be stable in alkaline conditions or at elevated temperature. The polymer or gel may have mechanical properties similar to the mechanical properties of the bead. For instance, the polymer or gel may be of a similar size to the bead. The polymer or gel may have a mechanical strength (e.g. tensile strength) similar to that of the bead. The polymer or gel may be of a lower density than an oil. The polymer or gel may be of a density that is roughly similar to that of a buffer. The polymer or gel may have a tunable pore size. The pore size may be chosen to, for instance, retain denatured nucleic acids. The pore size may be chosen to maintain diffusive permeability to exogenous chemicals such as sodium hydroxide (NaOH) and/or endogenous chemicals such as inhibitors. The polymer or gel may be biocompatible. The polymer or gel may maintain or enhance cell viability. The polymer or gel may be biochemically compatible. The polymer or gel may be polymerized and/or depolymerized thermally, chemically, enzymatically, and/or optically.

The polymer may comprise poly(acrylamide-co-acrylic acid) crosslinked with disulfide linkages. The preparation of the polymer may comprise a two-step reaction. In the first activation step, poly(acrylamide-co-acrylic acid) may be exposed to an acylating agent to convert carboxylic acids to esters. For instance, the poly(acrylamide-co-acrylic acid) may be exposed to 4-(4,6-dimethoxy-1,3,5-triazin-2-yl)-4-methylmorpholinium chloride (DMTMM). The polyacrylamide-co-acrylic acid may be exposed to other salts of 4-(4,6-dimethoxy-1,3,5-triazin-2-yl)-4-methylmorpholinium. In the second cross-linking step, the ester formed in the first step may be exposed to a disulfide crosslinking agent. For instance, the ester may be exposed to cystamine (2,2′-dithiobis(ethylamine)). Following the two steps, the biological particle may be surrounded by polyacrylamide strands linked together by disulfide bridges. In this manner, the biological particle may be encased inside of or comprise a gel or matrix (e.g., polymer matrix) to form a “cell bead.” A cell bead can contain biological particles (e.g., a virus, a cell, or a cell nucleus) or macromolecular constituents (e.g., RNA, DNA, proteins, etc.) of biological particles. For example after lysing and washing the cells, inhibitory components from cell lysates can be washed away and the macromolecular constituents can be bound as cell beads. Systems and methods disclosed herein can be applicable to both cell beads (and/or droplets or other partitions) containing biological particles and cell beads (and/or droplets or other partitions) containing macromolecular constituents of biological particles.

A cell bead may include a single cell or multiple cells, or a derivative of the single cell or multiple cells (e.g., multiple cells adhered together). A cell bead may include any type of cell, including without limitation prokaryotic cells, eukaryotic cells, bacterial, fungal, plant, mammalian, or other animal cell types, mycoplasmas, normal tissue cells, tumor cells, a T-cell (e.g., CD4 T-cell, CD4 T-cell that comprises a dormant copy of human immunodeficiency virus (HIV)), a fixed cell, a cross-linked cell, a rare cell from a population of cells, or any other cell type, whether derived from single cell or multicellular organisms. Furthermore, a cell bead may comprise a live cell, such as, for example, a cell may be capable of being cultured. Moreover, in some examples, a cell bead may comprise a derivative of a cell, such as one or more components of the cell (e.g., an organelle, a cell protein, a cellular nucleic acid, genomic nucleic acid, messenger ribonucleic acid, a ribosome, a cellular enzyme, etc.). In some examples, a cell bead may comprise material obtained from a biological tissue, such as, for example, obtained from a subject. In some cases, cells, viruses or macromolecular constituents thereof are encapsulated within a cell bead. Encapsulation can be within a polymer or gel matrix that forms a structural component of the cell bead. In some cases, a cell bead is generated by fixing a cell in a fixation medium or by cross-linking elements of the cell, such as the cell membrane, the cell cytoskeleton, etc. In some cases, beads may or may not be generated without encapsulation within a larger cell bead.

Encapsulated biological particles can provide certain potential advantages of being more storable and more portable than droplet-based partitioned biological particles. Furthermore, in some cases, it may be desirable to allow biological particles to incubate for a select period of time before analysis, such as in order to characterize changes in such biological particles over time, either in the presence or absence of different stimuli. In such cases, encapsulation may allow for longer incubation than partitioning in emulsion droplets, although in some cases, droplet partitioned biological particles may also be incubated for different periods of time, e.g., at least 10 seconds, at least 30 seconds, at least 1 minute, at least 5 minutes, at least 10 minutes, at least 30 minutes, at least 1 hour, at least 2 hours, at least 5 hours, or at least 10 hours or more. The encapsulation of biological particles may constitute the partitioning of the biological particles into which other reagents are co-partitioned. Alternatively or in addition, encapsulated biological particles may be readily deposited into other partitions (e.g., droplets) as described above.

### Beads

A partition may comprise one or more unique identifiers, such as barcodes. Barcodes may be previously, subsequently or concurrently delivered to the partitions that hold the compartmentalized or partitioned biological particle. For example, barcodes may be injected into droplets previous to, subsequent to, or concurrently with droplet generation. The delivery of the barcodes to a particular partition allows for the later attribution of the characteristics of the individual biological particle to the particular partition. Barcodes may be delivered, for example on a nucleic acid molecule (e.g., an oligonucleotide), to a partition via any suitable mechanism. Barcoded nucleic acid molecules can be delivered to a partition via a microcapsule. A microcapsule, in some instances, can comprise a bead. Beads are described in further detail below.

In some cases, barcoded nucleic acid molecules can be initially associated with the microcapsule and then released from the microcapsule. Release of the barcoded nucleic acid molecules can be passive (e.g., by diffusion out of the microcapsule). In addition or alternatively, release from the microcapsule can be upon application of a stimulus which allows the barcoded nucleic acid nucleic acid molecules to dissociate or to be released from the microcapsule. Such stimulus may disrupt the microcapsule, an interaction that couples the barcoded nucleic acid molecules to or within the microcapsule, or both. Such stimulus can include, for example, a thermal stimulus, photo-stimulus, chemical stimulus (e.g., change in pH or use of a reducing agent(s)), a mechanical stimulus, a radiation stimulus; a biological stimulus (e.g., enzyme), or any combination thereof.

FIG. 2 shows an example of a microfluidic channel structure 200 for delivering barcode carrying beads to droplets. The channel structure 200 can include channel segments 201, 202, 204, 206 and 208 communicating at a channel junction 210. In operation, the channel segment 201 may transport an aqueous fluid 212 that includes a plurality of beads 214 (e.g., with nucleic acid molecules, oligonucleotides, molecular tags) along the channel segment 201 into junction 210. The plurality of beads 214 may be sourced from a suspension of beads. For example, the channel segment 201 may be connected to a reservoir comprising an aqueous suspension of beads 214. The channel segment 202 may transport the aqueous fluid 212 that includes a plurality of biological particles 216 along the channel segment 202 into junction 210. The plurality of biological particles 216 may be sourced from a suspension of biological particles. For example, the channel segment 202 may be connected to a reservoir comprising an aqueous suspension of biological particles 216. In some instances, the aqueous fluid 212 in either the first channel segment 201 or the second channel segment 202, or in both segments, can include one or more reagents, as further described below. A second fluid 218 that is immiscible with the aqueous fluid 212 (e.g., oil) can be delivered to the junction 210 from each of channel segments 204 and 206. Upon meeting of the aqueous fluid 212 from each of channel segments 201 and 202 and the second fluid 218 from each of channel segments 204 and 206 at the channel junction 210, the aqueous fluid 212 can be partitioned as discrete droplets 220 in the second fluid 218 and flow away from the junction 210 along channel segment 208. The channel segment 208 may deliver the discrete droplets to an outlet reservoir fluidly coupled to the channel segment 208, where they may be harvested.

As an alternative, the channel segments 201 and 202 may meet at another junction upstream of the junction 210. At such junction, beads and biological particles may form a mixture that is directed along another channel to the junction 210 to yield droplets 220. The mixture may provide the beads and biological particles in an alternating fashion, such that, for example, a droplet comprises a single bead and a single biological particle.

Beads, biological particles and droplets may flow along channels at substantially regular flow profiles (e.g., at regular flow rates). Such regular flow profiles may permit a droplet to include a single bead and a single biological particle. Such regular flow profiles may permit the droplets to have an occupancy (e.g., droplets having beads and biological particles) greater than 5%, 10%, 20%, 30%, 40%, 50%, 60%, 70%, 80%, 90%, or 95%. Such regular flow profiles and devices that may be used to provide such regular flow profiles are provided in, for example, U.S. Patent Publication No. 2015/0292988, which is entirely incorporated herein by reference.

The second fluid 218 can comprise an oil, such as a fluorinated oil, that includes a fluorosurfactant for stabilizing the resulting droplets, for example, inhibiting subsequent coalescence of the resulting droplets 220.

A discrete droplet that is generated may include an individual biological particle 216. A discrete droplet that is generated may include a barcode or other reagent carrying bead 214. A discrete droplet generated may include both an individual biological particle and a barcode carrying bead, such as droplets 220. In some instances, a discrete droplet may include more than one individual biological particle or no biological particle. In some instances, a discrete droplet may include more than one bead or no bead. A discrete droplet may be unoccupied (e.g., no beads, no biological particles).

Beneficially, a discrete droplet partitioning a biological particle and a barcode carrying bead may effectively allow the attribution of the barcode to macromolecular constituents of the biological particle within the partition. The contents of a partition may remain discrete from the contents of other partitions.

As will be appreciated, the channel segments described herein may be coupled to any of a variety of different fluid sources or receiving components, including reservoirs, tubing, manifolds, or fluidic components of other systems. As will be appreciated, the microfluidic channel structure 200 may have other geometries. For example, a microfluidic channel structure can have more than one channel junctions. For example, a microfluidic channel structure can have 2, 3, 4, or 5 channel segments each carrying beads that meet at a channel junction. Fluid may be directed flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electrokinetic pumping, vacuum, capillary or gravity flow, or the like.

A bead may be porous, non-porous, solid, semi-solid, semi-fluidic, fluidic, and/or a combination thereof. In some instances, a bead may be dissolvable, disruptable, and/or degradable. In some cases, a bead may not be degradable. In some cases, the bead may be a gel bead. A gel bead may be a hydrogel bead. A gel bead may be formed from molecular precursors, such as a polymeric or monomeric species. A semi-solid bead may be a liposomal bead. Solid beads may comprise metals including iron oxide, gold, and silver. In some cases, the bead may be a silica bead. In some cases, the bead can be rigid. In other cases, the bead may be flexible and/or compressible.

A bead may be of any suitable shape. Examples of bead shapes include, but are not limited to, spherical, non-spherical, oval, oblong, amorphous, circular, cylindrical, and variations thereof.

Beads may be of uniform size or heterogeneous size. In some cases, the diameter of a bead may be at least about 10 nanometers (nm), 100 nm, 500 nm, 1 micrometer (μm), 5 μm, 10 μm, 20 μm, 30 μm, 40 μm, 50 μm, 60 μm, 70 μm, 80 μm, 90 μm, 100 μm, 250 μm, 500 μm, 1 mm, or greater. In some cases, a bead may have a diameter of less than about 10 nm, 100 nm, 500 nm, 1 μm, 5 μm, 10 μm, 20 μm, 30 μm, 40 μm, 50 μm, 60 μm, 70 μm, 80 μm, 90 μm, 100 μm, 250 μm, 500 μm, 1 mm, or less. In some cases, a bead may have a diameter in the range of about 40-75 μm, 30-75 μm, 20-75 μm, 40-85 μm, 40-95 μm, 20-100 μm, 10-100 μm, 1-100 μm, 20-250 μm, or 20-500 μm.

In certain aspects, beads can be provided as a population or plurality of beads having a relatively monodisperse size distribution. Where it may be desirable to provide relatively consistent amounts of reagents within partitions, maintaining relatively consistent bead characteristics, such as size, can contribute to the overall consistency. In particular, the beads described herein may have size distributions that have a coefficient of variation in their cross-sectional dimensions of less than 50%, less than 40%, less than 30%, less than 20%, and in some cases less than 15%, less than 10%, less than 5%, or less.

A bead may comprise natural and/or synthetic materials. For example, a bead can comprise a natural polymer, a synthetic polymer or both natural and synthetic polymers. Examples of natural polymers include proteins and sugars such as deoxyribonucleic acid, rubber, cellulose, starch (e.g., amylose, amylopectin), proteins, enzymes, polysaccharides, silks, polyhydroxyalkanoates, chitosan, dextran, collagen, carrageenan, ispaghula, acacia, agar, gelatin, shellac, sterculia gum, xanthan gum, Corn sugar gum, guar gum, gum karaya, agarose, alginic acid, alginate, or natural polymers thereof. Examples of synthetic polymers include acrylics, nylons, silicones, spandex, viscose rayon, polycarboxylic acids, polyvinyl acetate, polyacrylamide, polyacrylate, polyethylene glycol, polyurethanes, polylactic acid, silica, polystyrene, polyacrylonitrile, polybutadiene, polycarbonate, polyethylene, polyethylene terephthalate, poly(chlorotrifluoroethylene), poly(ethylene oxide), poly(ethylene terephthalate), polyethylene, polyisobutylene, poly(methyl methacrylate), poly(oxymethylene), polyformaldehyde, polypropylene, polystyrene, poly(tetrafluoroethylene), poly(vinyl acetate), poly(vinyl alcohol), poly(vinyl chloride), poly(vinylidene dichloride), poly(vinylidene difluoride), poly(vinyl fluoride) and/or combinations (e.g., co-polymers) thereof. Beads may also be formed from materials other than polymers, including lipids, micelles, ceramics, glass-ceramics, material composites, metals, other inorganic materials, and others.

In some instances, the bead may contain molecular precursors (e.g., monomers or polymers), which may form a polymer network via polymerization of the molecular precursors. In some cases, a precursor may be an already polymerized species capable of undergoing further polymerization via, for example, a chemical cross-linkage. In some cases, a precursor can comprise one or more of an acrylamide or a methacrylamide monomer, oligomer, or polymer. In some cases, the bead may comprise prepolymers, which are oligomers capable of further polymerization. For example, polyurethane beads may be prepared using prepolymers. In some cases, the bead may contain individual polymers that may be further polymerized together. In some cases, beads may be generated via polymerization of different precursors, such that they comprise mixed polymers, co-polymers, and/or block co-polymers. In some cases, the bead may comprise covalent or ionic bonds between polymeric precursors (e.g., monomers, oligomers, linear polymers), nucleic acid molecules (e.g., oligonucleotides), primers, and other entities. In some cases, the covalent bonds can be carbon-carbon bonds, thioether bonds, or carbon-heteroatom bonds.

Cross-linking may be permanent or reversible, depending upon the particular cross-linker used. Reversible cross-linking may allow for the polymer to linearize or dissociate under appropriate conditions. In some cases, reversible cross-linking may also allow for reversible attachment of a material bound to the surface of a bead. In some cases, a cross-linker may form disulfide linkages. In some cases, the chemical cross-linker forming disulfide linkages may be cystamine or a modified cystamine.

In some cases, disulfide linkages can be formed between molecular precursor units (e.g., monomers, oligomers, or linear polymers) or precursors incorporated into a bead and nucleic acid molecules (e.g., oligonucleotides). Cystamine (including modified cystamines), for example, is an organic agent comprising a disulfide bond that may be used as a crosslinker agent between individual monomeric or polymeric precursors of a bead. Polyacrylamide may be polymerized in the presence of cystamine or a species comprising cystamine (e.g., a modified cystamine) to generate polyacrylamide gel beads comprising disulfide linkages (e.g., chemically degradable beads comprising chemically-reducible cross-linkers). The disulfide linkages may permit the bead to be degraded (or dissolved) upon exposure of the bead to a reducing agent.

In some cases, chitosan, a linear polysaccharide polymer, may be crosslinked with glutaraldehyde via hydrophilic chains to form a bead. Crosslinking of chitosan polymers may be achieved by chemical reactions that are initiated by heat, pressure, change in pH, and/or radiation.

In some cases, a bead may comprise an acrydite moiety, which in certain aspects may be used to attach one or more nucleic acid molecules (e.g., barcode sequence, barcoded nucleic acid molecule, barcoded oligonucleotide, primer, or other oligonucleotide) to the bead. In some cases, an acrydite moiety can refer to an acrydite analogue generated from the reaction of acrydite with one or more species, such as, the reaction of acrydite with other monomers and cross-linkers during a polymerization reaction. Acrydite moieties may be modified to form chemical bonds with a species to be attached, such as a nucleic acid molecule (e.g., barcode sequence, barcoded nucleic acid molecule, barcoded oligonucleotide, primer, or other oligonucleotide). Acrydite moieties may be modified with thiol groups capable of forming a disulfide bond or may be modified with groups already comprising a disulfide bond. The thiol or disulfide (via disulfide exchange) may be used as an anchor point for a species to be attached or another part of the acrydite moiety may be used for attachment. In some cases, attachment can be reversible, such that when the disulfide bond is broken (e.g., in the presence of a reducing agent), the attached species is released from the bead. In other cases, an acrydite moiety can comprise a reactive hydroxyl group that may be used for attachment.

Functionalization of beads for attachment of nucleic acid molecules (e.g., oligonucleotides) may be achieved through a wide range of different approaches, including activation of chemical groups within a polymer, incorporation of active or activatable functional groups in the polymer structure, or attachment at the pre-polymer or monomer stage in bead production.

For example, precursors (e.g., monomers, cross-linkers) that are polymerized to form a bead may comprise acrydite moieties, such that when a bead is generated, the bead also comprises acrydite moieties. The acrydite moieties can be attached to a nucleic acid molecule (e.g., oligonucleotide), which may include a priming sequence (e.g., a primer for amplifying target nucleic acids, random primer, primer sequence for messenger RNA) and/or one or more barcode sequences. The one more barcode sequences may include sequences that are the same for all nucleic acid molecules coupled to a given bead and/or sequences that are different across all nucleic acid molecules coupled to the given bead. The nucleic acid molecule may be incorporated into the bead.

In some cases, the nucleic acid molecule can comprise a functional sequence, for example, for attachment to a sequencing flow cell, such as, for example, a P5 sequence for Illumina® sequencing. In some cases, the nucleic acid molecule or derivative thereof (e.g., oligonucleotide or polynucleotide generated from the nucleic acid molecule) can comprise another functional sequence, such as, for example, a P7 sequence for attachment to a sequencing flow cell for Illumina sequencing. In some cases, the nucleic acid molecule can comprise a barcode sequence. In some cases, the primer can further comprise a unique molecular identifier (UMI). In some cases, the primer can comprise an R1 primer sequence for Illumina sequencing. In some cases, the primer can comprise an R2 primer sequence for Illumina sequencing. Examples of such nucleic acid molecules (e.g., oligonucleotides, polynucleotides, etc.) and uses thereof, as may be used with compositions, devices, methods and systems of the present disclosure, are provided in U.S. Patent Pub. Nos. 2014/0378345 and 2015/0376609, each of which is entirely incorporated herein by reference.

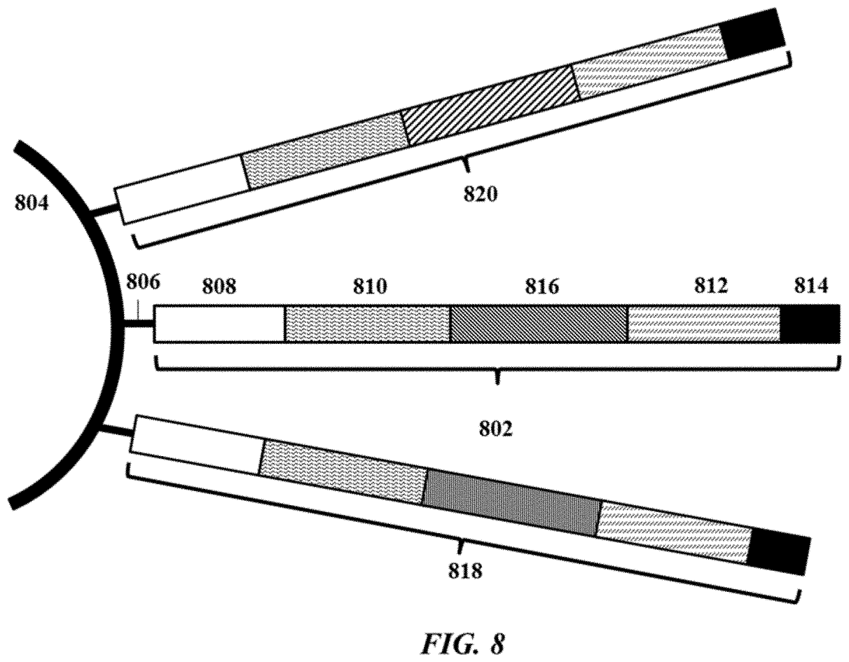

FIG. 8 illustrates an example of a barcode carrying bead. A nucleic acid molecule 802, such as an oligonucleotide, can be coupled to a bead 804 by a releasable linkage 806, such as, for example, a disulfide linker. The same bead 804 may be coupled (e.g., via releasable linkage) to one or more other nucleic acid molecules 818, 820. The nucleic acid molecule 802 may be or comprise a barcode. As noted elsewhere herein, the structure of the barcode may comprise a number of sequence elements. The nucleic acid molecule 802 may comprise a functional sequence 808 that may be used in subsequent processing. For example, the functional sequence 808 may include one or more of a sequencer specific flow cell attachment sequence (e.g., a P5 sequence for Illumina® sequencing systems) and a sequencing primer sequence (e.g., a R1 primer for Illumina® sequencing systems). The nucleic acid molecule 802 may comprise a barcode sequence 810 for use in barcoding the sample (e.g., DNA, RNA, protein, etc.). In some cases, the barcode sequence 810 can be bead-specific such that the barcode sequence 810 is common to all nucleic acid molecules (e.g., including nucleic acid molecule 802) coupled to the same bead 804. Alternatively or in addition, the barcode sequence 810 can be partition-specific such that the barcode sequence 810 is common to all nucleic acid molecules coupled to one or more beads that are partitioned into the same partition. The nucleic acid molecule 802 may comprise a specific priming sequence 812, such as an mRNA specific priming sequence (e.g., poly-T sequence), a targeted priming sequence, and/or a random priming sequence. The nucleic acid molecule 802 may comprise an anchoring sequence 814 to ensure that the specific priming sequence 812 hybridizes at the sequence end (e.g., of the mRNA). For example, the anchoring sequence 814 can include a random short sequence of nucleotides, such as a 1-mer, 2-mer, 3-mer or longer sequence, which can ensure that a poly-T segment is more likely to hybridize at the sequence end of the poly-A tail of the mRNA.

The nucleic acid molecule 802 may comprise a unique molecular identifying sequence 816 (e.g., unique molecular identifier (UMI)). In some cases, the unique molecular identifying sequence 816 may comprise from about 5 to about 8 nucleotides. Alternatively, the unique molecular identifying sequence 816 may compress less than about 5 or more than about 8 nucleotides. The unique molecular identifying sequence 816 may be a unique sequence that varies across individual nucleic acid molecules (e.g., 802, 818, 820, etc.) coupled to a single bead (e.g., bead 804). In some cases, the unique molecular identifying sequence 816 may be a random sequence (e.g., such as a random N-mer sequence). For example, the UMI may provide a unique identifier of the starting mRNA molecule that was captured, in order to allow quantitation of the number of original expressed RNA. As will be appreciated, although FIG. 8 shows three nucleic acid molecules 802, 818, 820 coupled to the surface of the bead 804, an individual bead may be coupled to any number of individual nucleic acid molecules, for example, from one to tens to hundreds of thousands or even millions of individual nucleic acid molecules. The respective barcodes for the individual nucleic acid molecules can comprise both common sequence segments or relatively common sequence segments (e.g., 808, 810, 812, etc.) and variable or unique sequence segments (e.g., 816) between different individual nucleic acid molecules coupled to the same bead.

In operation, a biological particle (e.g., cell, DNA, RNA, etc.) can be co-partitioned along with a barcode bearing bead 804. The barcoded nucleic acid molecules 802, 818, 820 can be released from the bead 804 in the partition. By way of example, in the context of analyzing sample RNA, the poly-T segment (e.g., 812) of one of the released nucleic acid molecules (e.g., 802) can hybridize to the poly-A tail of a mRNA molecule. Reverse transcription may result in a cDNA transcript of the mRNA, but which transcript includes each of the sequence segments 808, 810, 816 of the nucleic acid molecule 802. Because the nucleic acid molecule 802 comprises an anchoring sequence 814, it will more likely hybridize to and prime reverse transcription at the sequence end of the poly-A tail of the mRNA. Within any given partition, all of the cDNA transcripts of the individual mRNA molecules may include a common barcode sequence segment 810. However, the transcripts made from the different mRNA molecules within a given partition may vary at the unique molecular identifying sequence 812 segment (e.g., UMI segment). Beneficially, even following any subsequent amplification of the contents of a given partition, the number of different UMIs can be indicative of the quantity of mRNA originating from a given partition, and thus from the biological particle (e.g., cell). As noted above, the transcripts can be amplified, cleaned up and sequenced to identify the sequence of the cDNA transcript of the mRNA, as well as to sequence the barcode segment and the UMI segment. While a poly-T primer sequence is described, other targeted or random priming sequences may also be used in priming the reverse transcription reaction. Likewise, although described as releasing the barcoded oligonucleotides into the partition, in some cases, the nucleic acid molecules bound to the bead (e.g., gel bead) may be used to hybridize and capture the mRNA on the solid phase of the bead, for example, in order to facilitate the separation of the RNA from other cell contents.

In some cases, precursors comprising a functional group that is reactive or capable of being activated such that it becomes reactive can be polymerized with other precursors to generate gel beads comprising the activated or activatable functional group. The functional group may then be used to attach additional species (e.g., disulfide linkers, primers, other oligonucleotides, etc.) to the gel beads. For example, some precursors comprising a carboxylic acid (COOH) group can co-polymerize with other precursors to form a gel bead that also comprises a COOH functional group. In some cases, acrylic acid (a species comprising free COOH groups), acrylamide, and bis(acryloyl)cystamine can be co-polymerized together to generate a gel bead comprising free COOH groups. The COOH groups of the gel bead can be activated (e.g., via 1-Ethyl-3-(3-dimethylaminopropyl)carbodiimide (EDC) and N-Hydroxysuccinimide (NHS) or 4-(4,6-Dimethoxy-1,3,5-triazin-2-yl)-4-methylmorpholinium chloride (DMTMM)) such that they are reactive (e.g., reactive to amine functional groups where EDC/NHS or DMTMM are used for activation). The activated COOH groups can then react with an appropriate species (e.g., a species comprising an amine functional group where the carboxylic acid groups are activated to be reactive with an amine functional group) comprising a moiety to be linked to the bead.

Beads comprising disulfide linkages in their polymeric network may be functionalized with additional species via reduction of some of the disulfide linkages to free thiols. The disulfide linkages may be reduced via, for example, the action of a reducing agent (e.g., DTT, TCEP, etc.) to generate free thiol groups, without dissolution of the bead. Free thiols of the beads can then react with free thiols of a species or a species comprising another disulfide bond (e.g., via thiol-disulfide exchange) such that the species can be linked to the beads (e.g., via a generated disulfide bond). In some cases, free thiols of the beads may react with any other suitable group. For example, free thiols of the beads may react with species comprising an acrydite moiety. The free thiol groups of the beads can react with the acrydite via Michael addition chemistry, such that the species comprising the acrydite is linked to the bead. In some cases, uncontrolled reactions can be prevented by inclusion of a thiol capping agent such as N-ethylmalieamide or iodoacetate.

Activation of disulfide linkages within a bead can be controlled such that only a small number of disulfide linkages are activated. Control may be exerted, for example, by controlling the concentration of a reducing agent used to generate free thiol groups and/or concentration of reagents used to form disulfide bonds in bead polymerization. In some cases, a low concentration (e.g., molecules of reducing agent:gel bead ratios of less than or equal to about 1:100,000,000,000, less than or equal to about 1:10,000,000,000, less than or equal to about 1:1,000,000,000, less than or equal to about 1:100,000,000, less than or equal to about 1:10,000,000, less than or equal to about 1:1,000,000, less than or equal to about 1:100,000, less than or equal to about 1:10,000) of reducing agent may be used for reduction. Controlling the number of disulfide linkages that are reduced to free thiols may be useful in ensuring bead structural integrity during functionalization. In some cases, optically-active agents, such as fluorescent dyes may be coupled to beads via free thiol groups of the beads and used to quantify the number of free thiols present in a bead and/or track a bead.

In some cases, addition of moieties to a gel bead after gel bead formation may be advantageous. For example, addition of an oligonucleotide (e.g., barcoded oligonucleotide) after gel bead formation may avoid loss of the species during chain transfer termination that can occur during polymerization. Moreover, smaller precursors (e.g., monomers or cross linkers that do not comprise side chain groups and linked moieties) may be used for polymerization and can be minimally hindered from growing chain ends due to viscous effects. In some cases, functionalization after gel bead synthesis can minimize exposure of species (e.g., oligonucleotides) to be loaded with potentially damaging agents (e.g., free radicals) and/or chemical environments. In some cases, the generated gel may possess an upper critical solution temperature (UCST) that can permit temperature driven swelling and collapse of a bead. Such functionality may aid in oligonucleotide (e.g., a primer) infiltration into the bead during subsequent functionalization of the bead with the oligonucleotide. Post-production functionalization may also be useful in controlling loading ratios of species in beads, such that, for example, the variability in loading ratio is minimized. Species loading may also be performed in a batch process such that a plurality of beads can be functionalized with the species in a single batch.

A bead injected or otherwise introduced into a partition may comprise releasably, cleavably, or reversibly attached barcodes. A bead injected or otherwise introduced into a partition may comprise activatable barcodes. A bead injected or otherwise introduced into a partition may be degradable, disruptable, or dissolvable beads.

Barcodes can be releasably, cleavably or reversibly attached to the beads such that barcodes can be released or be releasable through cleavage of a linkage between the barcode molecule and the bead, or released through degradation of the underlying bead itself, allowing the barcodes to be accessed or be accessible by other reagents, or both. In non-limiting examples, cleavage may be achieved through reduction of di-sulfide bonds, use of restriction enzymes, photo-activated cleavage, or cleavage via other types of stimuli (e.g., chemical, thermal, pH, enzymatic, etc.) and/or reactions, such as described elsewhere herein. Releasable barcodes may sometimes be referred to as being activatable, in that they are available for reaction once released. Thus, for example, an activatable barcode may be activated by releasing the barcode from a bead (or other suitable type of partition described herein). Other activatable configurations are also envisioned in the context of the described methods and systems.

In addition to, or as an alternative to the cleavable linkages between the beads and the associated molecules, such as barcode containing nucleic acid molecules (e.g., barcoded oligonucleotides), the beads may be degradable, disruptable, or dissolvable spontaneously or upon exposure to one or more stimuli (e.g., temperature changes, pH changes, exposure to particular chemical species or phase, exposure to light, reducing agent, etc.). In some cases, a bead may be dissolvable, such that material components of the beads are solubilized when exposed to a particular chemical species or an environmental change, such as a change temperature or a change in pH. In some cases, a gel bead can be degraded or dissolved at elevated temperature and/or in basic conditions. In some cases, a bead may be thermally degradable such that when the bead is exposed to an appropriate change in temperature (e.g., heat), the bead degrades. Degradation or dissolution of a bead bound to a species (e.g., a nucleic acid molecule, e.g., barcoded oligonucleotide) may result in release of the species from the bead.

As will be appreciated from the above disclosure, the degradation of a bead may refer to the disassociation of a bound or entrained species from a bead, both with and without structurally degrading the physical bead itself. For example, the degradation of the bead may involve cleavage of a cleavable linkage via one or more species and/or methods described elsewhere herein. In another example, entrained species may be released from beads through osmotic pressure differences due to, for example, changing chemical environments. By way of example, alteration of bead pore sizes due to osmotic pressure differences can generally occur without structural degradation of the bead itself In some cases, an increase in pore size due to osmotic swelling of a bead can permit the release of entrained species within the bead. In other cases, osmotic shrinking of a bead may cause a bead to better retain an entrained species due to pore size contraction.

A degradable bead may be introduced into a partition, such as a droplet of an emulsion or a well, such that the bead degrades within the partition and any associated species (e.g., oligonucleotides) are released within the droplet when the appropriate stimulus is applied. The free species (e.g., oligonucleotides, nucleic acid molecules) may interact with other reagents contained in the partition. For example, a polyacrylamide bead comprising cystamine and linked, via a disulfide bond, to a barcode sequence, may be combined with a reducing agent within a droplet of a water-in-oil emulsion. Within the droplet, the reducing agent can break the various disulfide bonds, resulting in bead degradation and release of the barcode sequence into the aqueous, inner environment of the droplet. In another example, heating of a droplet comprising a bead-bound barcode sequence in basic solution may also result in bead degradation and release of the attached barcode sequence into the aqueous, inner environment of the droplet.

Any suitable number of molecular tag molecules (e.g., primer, barcoded oligonucleotide) can be associated with a bead such that, upon release from the bead, the molecular tag molecules (e.g., primer, e.g., barcoded oligonucleotide) are present in the partition at a pre-defined concentration. Such pre-defined concentration may be selected to facilitate certain reactions for generating a sequencing library, e.g., amplification, within the partition. In some cases, the pre-defined concentration of the primer can be limited by the process of producing nucleic acid molecule (e.g., oligonucleotide) bearing beads.

In some cases, beads can be non-covalently loaded with one or more reagents. The beads can be non-covalently loaded by, for instance, subjecting the beads to conditions sufficient to swell the beads, allowing sufficient time for the reagents to diffuse into the interiors of the beads, and subjecting the beads to conditions sufficient to de-swell the beads. The swelling of the beads may be accomplished, for instance, by placing the beads in a thermodynamically favorable solvent, subjecting the beads to a higher or lower temperature, subjecting the beads to a higher or lower ion concentration, and/or subjecting the beads to an electric field. The swelling of the beads may be accomplished by various swelling methods. The de-swelling of the beads may be accomplished, for instance, by transferring the beads in a thermodynamically unfavorable solvent, subjecting the beads to lower or high temperatures, subjecting the beads to a lower or higher ion concentration, and/or removing an electric field. The de-swelling of the beads may be accomplished by various de-swelling methods. Transferring the beads may cause pores in the bead to shrink. The shrinking may then hinder reagents within the beads from diffusing out of the interiors of the beads. The hindrance may be due to steric interactions between the reagents and the interiors of the beads. The transfer may be accomplished microfluidically. For instance, the transfer may be achieved by moving the beads from one co-flowing solvent stream to a different co-flowing solvent stream. The swellability and/or pore size of the beads may be adjusted by changing the polymer composition of the bead.

In some cases, an acrydite moiety linked to a precursor, another species linked to a precursor, or a precursor itself can comprise a labile bond, such as chemically, thermally, or photo-sensitive bond e.g., disulfide bond, UV sensitive bond, or the like. Once acrydite moieties or other moieties comprising a labile bond are incorporated into a bead, the bead may also comprise the labile bond. The labile bond may be, for example, useful in reversibly linking (e.g., covalently linking) species (e.g., barcodes, primers, etc.) to a bead. In some cases, a thermally labile bond may include a nucleic acid hybridization based attachment, e.g., where an oligonucleotide is hybridized to a complementary sequence that is attached to the bead, such that thermal melting of the hybrid releases the oligonucleotide, e.g., a barcode containing sequence, from the bead or microcapsule.

The addition of multiple types of labile bonds to a gel bead may result in the generation of a bead capable of responding to varied stimuli. Each type of labile bond may be sensitive to an associated stimulus (e.g., chemical stimulus, light, temperature, enzymatic, etc.) such that release of species attached to a bead via each labile bond may be controlled by the application of the appropriate stimulus. Such functionality may be useful in controlled release of species from a gel bead. In some cases, another species comprising a labile bond may be linked to a gel bead after gel bead formation via, for example, an activated functional group of the gel bead as described above. As will be appreciated, barcodes that are releasably, cleavably or reversibly attached to the beads described herein include barcodes that are released or releasable through cleavage of a linkage between the barcode molecule and the bead, or that are released through degradation of the underlying bead itself, allowing the barcodes to be accessed or accessible by other reagents, or both.

The barcodes that are releasable as described herein may sometimes be referred to as being activatable, in that they are available for reaction once released. Thus, for example, an activatable barcode may be activated by releasing the barcode from a bead (or other suitable type of partition described herein). Other activatable configurations are also envisioned in the context of the described methods and systems.

In addition to thermally cleavable bonds, disulfide bonds and UV sensitive bonds, other non-limiting examples of labile bonds that may be coupled to a precursor or bead include an ester linkage (e.g., cleavable with an acid, a base, or hydroxylamine), a vicinal diol linkage (e.g., cleavable via sodium periodate), a Diels-Alder linkage (e.g., cleavable via heat), a sulfone linkage (e.g., cleavable via a base), a silyl ether linkage (e.g., cleavable via an acid), a glycosidic linkage (e.g., cleavable via an amylase), a peptide linkage (e.g., cleavable via a protease), or a phosphodiester linkage (e.g., cleavable via a nuclease (e.g., DNase)). A bond may be cleavable via other nucleic acid molecule targeting enzymes, such as restriction enzymes (e.g., restriction endonucleases), as described further below.

Species may be encapsulated in beads during bead generation (e.g., during polymerization of precursors). Such species may or may not participate in polymerization. Such species may be entered into polymerization reaction mixtures such that generated beads comprise the species upon bead formation. In some cases, such species may be added to the gel beads after formation. Such species may include, for example, nucleic acid molecules (e.g., oligonucleotides), reagents for a nucleic acid amplification reaction (e.g., primers, polymerases, dNTPs, co-factors (e.g., ionic co-factors), buffers) including those described herein, reagents for enzymatic reactions (e.g., enzymes, co-factors, substrates, buffers), reagents for nucleic acid modification reactions such as polymerization, ligation, or digestion, and/or reagents for template preparation (e.g., tagmentation) for one or more sequencing platforms (e.g., Nextera® for Illumina®). Such species may include one or more enzymes described herein, including without limitation, polymerase, reverse transcriptase, restriction enzymes (e.g., endonuclease), transposase, ligase, proteinase K, DNase, etc. Such species may include one or more reagents described elsewhere herein (e.g., lysis agents, inhibitors, inactivating agents, chelating agents, stimulus). Trapping of such species may be controlled by the polymer network density generated during polymerization of precursors, control of ionic charge within the gel bead (e.g., via ionic species linked to polymerized species), or by the release of other species. Encapsulated species may be released from a bead upon bead degradation and/or by application of a stimulus capable of releasing the species from the bead. Alternatively or in addition, species may be partitioned in a partition (e.g., droplet) during or subsequent to partition formation. Such species may include, without limitation, the abovementioned species that may also be encapsulated in a bead.

A degradable bead may comprise one or more species with a labile bond such that, when the bead/species is exposed to the appropriate stimuli, the bond is broken and the bead degrades. The labile bond may be a chemical bond (e.g., covalent bond, ionic bond) or may be another type of physical interaction (e.g., van der Waals interactions, dipole-dipole interactions, etc.). In some cases, a crosslinker used to generate a bead may comprise a labile bond. Upon exposure to the appropriate conditions, the labile bond can be broken and the bead degraded. For example, upon exposure of a polyacrylamide gel bead comprising cystamine crosslinkers to a reducing agent, the disulfide bonds of the cystamine can be broken and the bead degraded.

A degradable bead may be useful in more quickly releasing an attached species (e.g., a nucleic acid molecule, a barcode sequence, a primer, etc) from the bead when the appropriate stimulus is applied to the bead as compared to a bead that does not degrade. For example, for a species bound to an inner surface of a porous bead or in the case of an encapsulated species, the species may have greater mobility and accessibility to other species in solution upon degradation of the bead. In some cases, a species may also be attached to a degradable bead via a degradable linker (e.g., disulfide linker). The degradable linker may respond to the same stimuli as the degradable bead or the two degradable species may respond to different stimuli. For example, a barcode sequence may be attached, via a disulfide bond, to a polyacrylamide bead comprising cystamine. Upon exposure of the barcoded-bead to a reducing agent, the bead degrades and the barcode sequence is released upon breakage of both the disulfide linkage between the barcode sequence and the bead and the disulfide linkages of the cystamine in the bead.

As will be appreciated from the above disclosure, while referred to as degradation of a bead, in many instances as noted above, that degradation may refer to the disassociation of a bound or entrained species from a bead, both with and without structurally degrading the physical bead itself. For example, entrained species may be released from beads through osmotic pressure differences due to, for example, changing chemical environments. By way of example, alteration of bead pore sizes due to osmotic pressure differences can generally occur without structural degradation of the bead itself In some cases, an increase in pore size due to osmotic swelling of a bead can permit the release of entrained species within the bead. In other cases, osmotic shrinking of a bead may cause a bead to better retain an entrained species due to pore size contraction.

Where degradable beads are provided, it may be beneficial to avoid exposing such beads to the stimulus or stimuli that cause such degradation prior to a given time, in order to, for example, avoid premature bead degradation and issues that arise from such degradation, including for example poor flow characteristics and aggregation. By way of example, where beads comprise reducible cross-linking groups, such as disulfide groups, it will be desirable to avoid contacting such beads with reducing agents, e.g., DTT or other disulfide cleaving reagents. In such cases, treatment to the beads described herein will, in some cases be provided free of reducing agents, such as DTT. Because reducing agents are often provided in commercial enzyme preparations, it may be desirable to provide reducing agent free (or DTT free) enzyme preparations in treating the beads described herein. Examples of such enzymes include, e.g., polymerase enzyme preparations, reverse transcriptase enzyme preparations, ligase enzyme preparations, as well as many other enzyme preparations that may be used to treat the beads described herein. The terms “reducing agent free” or “DTT free” preparations can refer to a preparation having less than about 1/10th, less than about 1/50th, or even less than about 1/100th of the lower ranges for such materials used in degrading the beads. For example, for DTT, the reducing agent free preparation can have less than about 0.01 millimolar (mM), 0.005 mM, 0.001 mM DTT, 0.0005 mM DTT, or even less than about 0.0001 mM DTT. In many cases, the amount of DTT can be undetectable.

Numerous chemical triggers may be used to trigger the degradation of beads. Examples of these chemical changes may include, but are not limited to pH-mediated changes to the integrity of a component within the bead, degradation of a component of a bead via cleavage of cross-linked bonds, and depolymerization of a component of a bead.

In some embodiments, a bead may be formed from materials that comprise degradable chemical crosslinkers, such as BAC or cystamine. Degradation of such degradable crosslinkers may be accomplished through a number of mechanisms. In some examples, a bead may be contacted with a chemical degrading agent that may induce oxidation, reduction or other chemical changes. For example, a chemical degrading agent may be a reducing agent, such as dithiothreitol (DTT). Additional examples of reducing agents may include β-mercaptoethanol, (2S)-2-amino-1,4-dimercaptobutane (dithiobutylamine or DTBA), tris(2-carboxyethyl) phosphine (TCEP), or combinations thereof. A reducing agent may degrade the disulfide bonds formed between gel precursors forming the bead, and thus, degrade the bead. In other cases, a change in pH of a solution, such as an increase in pH, may trigger degradation of a bead. In other cases, exposure to an aqueous solution, such as water, may trigger hydrolytic degradation, and thus degradation of the bead. In some cases, any combination of stimuli may trigger degradation of a bead. For example, a change in pH may enable a chemical agent (e.g., DTT) to become an effective reducing agent.

Beads may also be induced to release their contents upon the application of a thermal stimulus. A change in temperature can cause a variety of changes to a bead. For example, heat can cause a solid bead to liquefy. A change in heat may cause melting of a bead such that a portion of the bead degrades. In other cases, heat may increase the internal pressure of the bead components such that the bead ruptures or explodes. Heat may also act upon heat-sensitive polymers used as materials to construct beads.

Any suitable agent may degrade beads. In some embodiments, changes in temperature or pH may be used to degrade thermo-sensitive or pH-sensitive bonds within beads. In some embodiments, chemical degrading agents may be used to degrade chemical bonds within beads by oxidation, reduction or other chemical changes. For example, a chemical degrading agent may be a reducing agent, such as DTT, wherein DTT may degrade the disulfide bonds formed between a crosslinker and gel precursors, thus degrading the bead. In some embodiments, a reducing agent may be added to degrade the bead, which may or may not cause the bead to release its contents. Examples of reducing agents may include dithiothreitol (DTT), β-mercaptoethanol, (2S)-2-amino-1,4-dimercaptobutane (dithiobutylamine or DTBA), tris(2-carboxyethyl) phosphine (TCEP), or combinations thereof. The reducing agent may be present at a concentration of about 0.1 mM, 0.5 mM, 1 mM, 5 mM, or 10 mM. The reducing agent may be present at a concentration of at least about 0.1 mM, 0.5 mM, 1 mM, 5 mM, 10 mM, or greater than 10 mM. The reducing agent may be present at concentration of at most about 10 mM, 5 mM, 1 mM, 0.5 mM, 0.1 mM, or less.

Any suitable number of molecular tag molecules (e.g., primer, barcoded oligonucleotide) can be associated with a bead such that, upon release from the bead, the molecular tag molecules (e.g., primer, e.g., barcoded oligonucleotide) are present in the partition at a pre-defined concentration. Such pre-defined concentration may be selected to facilitate certain reactions for generating a sequencing library, e.g., amplification, within the partition. In some cases, the pre-defined concentration of the primer can be limited by the process of producing oligonucleotide bearing beads.

Although FIG. 1 and FIG. 2 have been described in terms of providing substantially singly occupied partitions, above, in certain cases, it may be desirable to provide multiply occupied partitions, e.g., containing two, three, four or more cells and/or microcapsules (e.g., beads) comprising barcoded nucleic acid molecules (e.g., oligonucleotides) within a single partition. Accordingly, as noted above, the flow characteristics of the biological particle and/or bead containing fluids and partitioning fluids may be controlled to provide for such multiply occupied partitions. In particular, the flow parameters may be controlled to provide a given occupancy rate at greater than about 50% of the partitions, greater than about 75%, and in some cases greater than about 80%, 90%, 95%, or higher.

In some cases, additional microcapsules can be used to deliver additional reagents to a partition. In such cases, it may be advantageous to introduce different beads into a common channel or droplet generation junction, from different bead sources (e.g., containing different associated reagents) through different channel inlets into such common channel or droplet generation junction (e.g., junction 210). In such cases, the flow and frequency of the different beads into the channel or junction may be controlled to provide for a certain ratio of microcapsules from each source, while ensuring a given pairing or combination of such beads into a partition with a given number of biological particles (e.g., one biological particle and one bead per partition).

The partitions described herein may comprise small volumes, for example, less than about 10 microliters (μL), 5 μL, 1 μL, 900 picoliters (pL), 800 pL, 700 pL, 600 pL, 500 pL, 400pL, 300 pL, 200 pL, 100pL, 50 pL, 20 pL, 10 pL, 1 pL, 500 nanoliters (nL), 100 nL, 50 nL, or less.

For example, in the case of droplet based partitions, the droplets may have overall volumes that are less than about 1000 pL, 900 pL, 800 pL, 700 pL, 600 pL, 500 pL, 400pL, 300 pL, 200 pL, 100pL, 50 pL, 20 pL, 10 pL, 1 pL, or less. Where co-partitioned with microcapsules, it will be appreciated that the sample fluid volume, e.g., including co-partitioned biological particles and/or beads, within the partitions may be less than about 90% of the above described volumes, less than about 80%, less than about 70%, less than about 60%, less than about 50%, less than about 40%, less than about 30%, less than about 20%, or less than about 10% of the above described volumes.

As is described elsewhere herein, partitioning species may generate a population or plurality of partitions. In such cases, any suitable number of partitions can be generated or otherwise provided. For example, at least about 1,000 partitions, at least about 5,000 partitions, at least about 10,000 partitions, at least about 50,000 partitions, at least about 100,000 partitions, at least about 500,000 partitions, at least about 1,000,000 partitions, at least about 5,000,000 partitions at least about 10,000,000 partitions, at least about 50,000,000 partitions, at least about 100,000,000 partitions, at least about 500,000,000 partitions, at least about 1,000,000,000 partitions, or more partitions can be generated or otherwise provided. Moreover, the plurality of partitions may comprise both unoccupied partitions (e.g., empty partitions) and occupied partitions.

### Reagents

In accordance with certain aspects, biological particles may be partitioned along with lysis reagents in order to release the contents of the biological particles within the partition. In such cases, the lysis agents can be contacted with the biological particle suspension concurrently with, or immediately prior to, the introduction of the biological particles into the partitioning junction/droplet generation zone (e.g., junction 210), such as through an additional channel or channels upstream of the channel junction. In accordance with other aspects, additionally or alternatively, biological particles may be partitioned along with other reagents, as will be described further below.

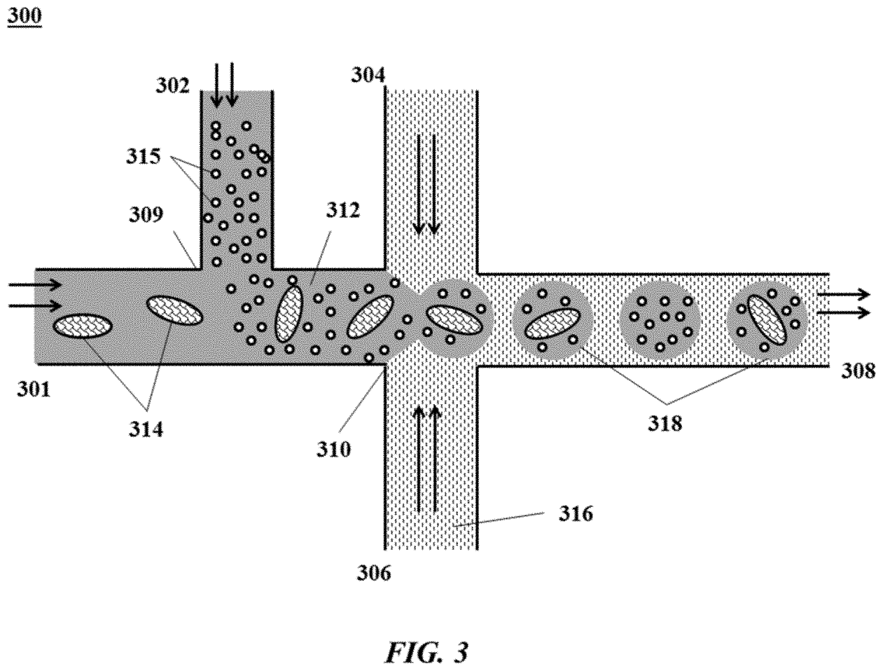

FIG. 3 shows an example of a microfluidic channel structure 300 for co-partitioning biological particles and reagents. The channel structure 300 can include channel segments 301, 302, 304, 306 and 308. Channel segments 301 and 302 communicate at a first channel junction 309. Channel segments 302, 304, 306, and 308 communicate at a second channel junction 310.

In an example operation, the channel segment 301 may transport an aqueous fluid 312 that includes a plurality of biological particles 314 along the channel segment 301 into the second junction 310. As an alternative or in addition to, channel segment 301 may transport beads (e.g., gel beads). The beads may comprise barcode molecules.

For example, the channel segment 301 may be connected to a reservoir comprising an aqueous suspension of biological particles 314. Upstream of, and immediately prior to reaching, the second junction 310, the channel segment 301 may meet the channel segment 302 at the first junction 309. The channel segment 302 may transport a plurality of reagents 315 (e.g., lysis agents) suspended in the aqueous fluid 312 along the channel segment 302 into the first junction 309. For example, the channel segment 302 may be connected to a reservoir comprising the reagents 315. After the first junction 309, the aqueous fluid 312 in the channel segment 301 can carry both the biological particles 314 and the reagents 315 towards the second junction 310. In some instances, the aqueous fluid 312 in the channel segment 301 can include one or more reagents, which can be the same or different reagents as the reagents 315. A second fluid 316 that is immiscible with the aqueous fluid 312 (e.g., oil) can be delivered to the second junction 310 from each of channel segments 304 and 306. Upon meeting of the aqueous fluid 312 from the channel segment 301 and the second fluid 316 from each of channel segments 304 and 306 at the second channel junction 310, the aqueous fluid 312 can be partitioned as discrete droplets 318 in the second fluid 316 and flow away from the second junction 310 along channel segment 308. The channel segment 308 may deliver the discrete droplets 318 to an outlet reservoir fluidly coupled to the channel segment 308, where they may be harvested.

The second fluid 316 can comprise an oil, such as a fluorinated oil, that includes a fluorosurfactant for stabilizing the resulting droplets, for example, inhibiting subsequent coalescence of the resulting droplets 318.

A discrete droplet generated may include an individual biological particle 314 and/or one or more reagents 315. In some instances, a discrete droplet generated may include a barcode carrying bead (not shown), such as via other microfluidics structures described elsewhere herein. In some instances, a discrete droplet may be unoccupied (e.g., no reagents, no biological particles).

Beneficially, when lysis reagents and biological particles are co-partitioned, the lysis reagents can facilitate the release of the contents of the biological particles within the partition. The contents released in a partition may remain discrete from the contents of other partitions.

As will be appreciated, the channel segments described herein may be coupled to any of a variety of different fluid sources or receiving components, including reservoirs, tubing, manifolds, or fluidic components of other systems. As will be appreciated, the microfluidic channel structure 300 may have other geometries. For example, a microfluidic channel structure can have more than two channel junctions. For example, a microfluidic channel structure can have 2, 3, 4, 5 channel segments or more each carrying the same or different types of beads, reagents, and/or biological particles that meet at a channel junction. Fluid flow in each channel segment may be controlled to control the partitioning of the different elements into droplets. Fluid may be directed flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electrokinetic pumping, vacuum, capillary or gravity flow, or the like.

Examples of lysis agents include bioactive reagents, such as lysis enzymes that are used for lysis of different cell types, e.g., gram positive or negative bacteria, plants, yeast, mammalian, etc., such as lysozymes, achromopeptidase, lysostaphin, labiase, kitalase, lyticase, and a variety of other lysis enzymes available from, e.g., Sigma-Aldrich, Inc. (St Louis, MO), as well as other commercially available lysis enzymes. Other lysis agents may additionally or alternatively be co-partitioned with the biological particles to cause the release of the biological particles' contents into the partitions. For example, in some cases, surfactant-based lysis solutions may be used to lyse cells, although these may be less desirable for emulsion based systems where the surfactants can interfere with stable emulsions. In some cases, lysis solutions may include non-ionic surfactants such as, for example, TritonX-100 and Tween 20. In some cases, lysis solutions may include ionic surfactants such as, for example, sarcosyl and sodium dodecyl sulfate (SDS). Electroporation, thermal, acoustic or mechanical cellular disruption may also be used in certain cases, e.g., non-emulsion based partitioning such as encapsulation of biological particles that may be in addition to or in place of droplet partitioning, where any pore size of the encapsulate is sufficiently small to retain nucleic acid fragments of a given size, following cellular disruption.

Alternatively or in addition to the lysis agents co-partitioned with the biological particles described above, other reagents can also be co-partitioned with the biological particles, including, for example, DNase and RNase inactivating agents or inhibitors, such as proteinase K, chelating agents, such as EDTA, and other reagents employed in removing or otherwise reducing negative activity or impact of different cell lysate components on subsequent processing of nucleic acids. In addition, in the case of encapsulated biological particles, the biological particles may be exposed to an appropriate stimulus to release the biological particles or their contents from a co-partitioned microcapsule. For example, in some cases, a chemical stimulus may be co-partitioned along with an encapsulated biological particle to allow for the degradation of the microcapsule and release of the cell or its contents into the larger partition. In some cases, this stimulus may be the same as the stimulus described elsewhere herein for release of nucleic acid molecules (e.g., oligonucleotides) from their respective microcapsule (e.g., bead). In alternative aspects, this may be a different and non-overlapping stimulus, in order to allow an encapsulated biological particle to be released into a partition at a different time from the release of nucleic acid molecules into the same partition.

Additional reagents may also be co-partitioned with the biological particles, such as endonucleases to fragment a biological particle's DNA, DNA polymerase enzymes and dNTPs used to amplify the biological particle's nucleic acid fragments and to attach the barcode molecular tags to the amplified fragments. Other enzymes may be co-partitioned, including without limitation, polymerase, transposase, ligase, proteinase K, DNase, etc. Additional reagents may also include reverse transcriptase enzymes, including enzymes with terminal transferase activity, primers and oligonucleotides, and switch oligonucleotides (also referred to herein as “switch oligos” or “template switching oligonucleotides”) which can be used for template switching. In some cases, template switching can be used to increase the length of a cDNA. In some cases, template switching can be used to append a predefined nucleic acid sequence to the cDNA. In an example of template switching, cDNA can be generated from reverse transcription of a template, e.g., cellular mRNA, where a reverse transcriptase with terminal transferase activity can add additional nucleotides, e.g., polyC, to the cDNA in a template independent manner. Switch oligos can include sequences complementary to the additional nucleotides, e.g., polyG. The additional nucleotides (e.g., polyC) on the cDNA can hybridize to the additional nucleotides (e.g., polyG) on the switch oligo, whereby the switch oligo can be used by the reverse transcriptase as template to further extend the cDNA. Template switching oligonucleotides may comprise a hybridization region and a template region. The hybridization region can comprise any sequence capable of hybridizing to the target. In some cases, as previously described, the hybridization region comprises a series of G bases to complement the overhanging C bases at the 3′ end of a cDNA molecule. The series of G bases may comprise 1 G base, 2 G bases, 3 G bases, 4 G bases, 5 G bases or more than 5 G bases. The template sequence can comprise any sequence to be incorporated into the cDNA. In some cases, the template region comprises at least 1 (e.g., at least 2, 3, 4, 5 or more) tag sequences and/or functional sequences. Switch oligos may comprise deoxyribonucleic acids; ribonucleic acids; modified nucleic acids including 2-Aminopurine, 2,6-Diaminopurine (2-Amino-dA), inverted dT, 5-Methyl dC, 2′-deoxyInosine, Super T (5-hydroxybutynl-2′-deoxyuridine), Super G (8-aza-7-deazaguanosine), locked nucleic acids (LNAs), unlocked nucleic acids (UNAs, e.g., UNA-A, UNA-U, UNA-C, UNA-G), Iso-dG, Iso-dC, 2′ Fluoro bases (e.g., Fluoro C, Fluoro U, Fluoro A, and Fluoro G), or any combination.

In some cases, the length of a switch oligo may be at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197 , 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249 or 250 nucleotides or longer.

In some cases, the length of a switch oligo may be at most about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197 , 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249 or 250 nucleotides.

Once the contents of the cells are released into their respective partitions, the macromolecular components (e.g., macromolecular constituents of biological particles, such as RNA, DNA, or proteins) contained therein may be further processed within the partitions. In accordance with the methods and systems described herein, the macromolecular component contents of individual biological particles can be provided with unique identifiers such that, upon characterization of those macromolecular components they may be attributed as having been derived from the same biological particle or particles. The ability to attribute characteristics to individual biological particles or groups of biological particles is provided by the assignment of unique identifiers specifically to an individual biological particle or groups of biological particles. Unique identifiers, e.g., in the form of nucleic acid barcodes can be assigned or associated with individual biological particles or populations of biological particles, in order to tag or label the biological particle's macromolecular components (and as a result, its characteristics) with the unique identifiers. These unique identifiers can then be used to attribute the biological particle's components and characteristics to an individual biological particle or group of biological particles.

In some aspects, this is performed by co-partitioning the individual biological particle or groups of biological particles with the unique identifiers, such as described above (with reference to FIG. 2). In some aspects, the unique identifiers are provided in the form of nucleic acid molecules (e.g., oligonucleotides) that comprise nucleic acid barcode sequences that may be attached to or otherwise associated with the nucleic acid contents of individual biological particle, or to other components of the biological particle, and particularly to fragments of those nucleic acids. The nucleic acid molecules are partitioned such that as between nucleic acid molecules in a given partition, the nucleic acid barcode sequences contained therein are the same, but as between different partitions, the nucleic acid molecule can, and do have differing barcode sequences, or at least represent a large number of different barcode sequences across all of the partitions in a given analysis. In some aspects, only one nucleic acid barcode sequence can be associated with a given partition, although in some cases, two or more different barcode sequences may be present.

The nucleic acid barcode sequences can include from about 6 to about 20 or more nucleotides within the sequence of the nucleic acid molecules (e.g., oligonucleotides). The nucleic acid barcode sequences can include from about 6 to about 20, 30, 40, 50, 60, 70, 80, 90, 100 or more nucleotides. In some cases, the length of a barcode sequence may be about 6, 7, 8, 9, 10, 11, 12, 13, 14, 15, 16, 17, 18, 19, 20 nucleotides or longer. In some cases, the length of a barcode sequence may be at least about 6, 7, 8, 9, 10, 11, 12, 13, 14, 15, 16, 17, 18, 19, 20 nucleotides or longer. In some cases, the length of a barcode sequence may be at most about 6, 7, 8, 9, 10, 11, 12, 13, 14, 15, 16, 17, 18, 19, 20 nucleotides or shorter. These nucleotides may be completely contiguous, i.e., in a single stretch of adjacent nucleotides, or they may be separated into two or more separate subsequences that are separated by 1 or more nucleotides. In some cases, separated barcode subsequences can be from about 4 to about 16 nucleotides in length. In some cases, the barcode subsequence may be about 4, 5, 6, 7, 8, 9, 10, 11, 12, 13, 14, 15, 16 nucleotides or longer. In some cases, the barcode subsequence may be at least about 4, 5, 6, 7, 8, 9, 10, 11, 12, 13, 14, 15, 16 nucleotides or longer. In some cases, the barcode subsequence may be at most about 4, 5, 6, 7, 8, 9, 10, 11, 12, 13, 14, 15, 16 nucleotides or shorter.

The co-partitioned nucleic acid molecules can also comprise other functional sequences useful in the processing of the nucleic acids from the co-partitioned biological particles. These sequences include, e.g., targeted or random/universal amplification primer sequences for amplifying the genomic DNA from the individual biological particles within the partitions while attaching the associated barcode sequences, sequencing primers or primer recognition sites, hybridization or probing sequences, e.g., for identification of presence of the sequences or for pulling down barcoded nucleic acids, or any of a number of other potential functional sequences. Other mechanisms of co-partitioning oligonucleotides may also be employed, including, e.g., coalescence of two or more droplets, where one droplet contains oligonucleotides, or microdispensing of oligonucleotides into partitions, e.g., droplets within microfluidic systems.

In an example, microcapsules, such as beads, are provided that each include large numbers of the above described barcoded nucleic acid molecules (e.g., barcoded oligonucleotides) releasably attached to the beads, where all of the nucleic acid molecules attached to a particular bead will include the same nucleic acid barcode sequence, but where a large number of diverse barcode sequences are represented across the population of beads used. In some embodiments, hydrogel beads, e.g., comprising polyacrylamide polymer matrices, are used as a solid support and delivery vehicle for the nucleic acid molecules into the partitions, as they are capable of carrying large numbers of nucleic acid molecules, and may be configured to release those nucleic acid molecules upon exposure to a particular stimulus, as described elsewhere herein. In some cases, the population of beads provides a diverse barcode sequence library that includes at least about 1,000 different barcode sequences, at least about 5,000 different barcode sequences, at least about 10,000 different barcode sequences, at least about 50,000 different barcode sequences, at least about 100,000 different barcode sequences, at least about 1,000,000 different barcode sequences, at least about 5,000,000 different barcode sequences, or at least about 10,000,000 different barcode sequences, or more. Additionally, each bead can be provided with large numbers of nucleic acid (e.g., oligonucleotide) molecules attached. In particular, the number of molecules of nucleic acid molecules including the barcode sequence on an individual bead can be at least about 1,000 nucleic acid molecules, at least about 5,000 nucleic acid molecules, at least about 10,000 nucleic acid molecules, at least about 50,000 nucleic acid molecules, at least about 100,000 nucleic acid molecules, at least about 500,000 nucleic acids, at least about 1,000,000 nucleic acid molecules, at least about 5,000,000 nucleic acid molecules, at least about 10,000,000 nucleic acid molecules, at least about 50,000,000 nucleic acid molecules, at least about 100,000,000 nucleic acid molecules, at least about 250,000,000 nucleic acid molecules and in some cases at least about 1 billion nucleic acid molecules, or more. Nucleic acid molecules of a given bead can include identical (or common) barcode sequences, different barcode sequences, or a combination of both. Nucleic acid molecules of a given bead can include multiple sets of nucleic acid molecules. Nucleic acid molecules of a given set can include identical barcode sequences. The identical barcode sequences can be different from barcode sequences of nucleic acid molecules of another set.

Moreover, when the population of beads is partitioned, the resulting population of partitions can also include a diverse barcode library that includes at least about 1,000 different barcode sequences, at least about 5,000 different barcode sequences, at least about 10,000 different barcode sequences, at least at least about 50,000 different barcode sequences, at least about 100,000 different barcode sequences, at least about 1,000,000 different barcode sequences, at least about 5,000,000 different barcode sequences, or at least about 10,000,000 different barcode sequences. Additionally, each partition of the population can include at least about 1,000 nucleic acid molecules, at least about 5,000 nucleic acid molecules, at least about 10,000 nucleic acid molecules, at least about 50,000 nucleic acid molecules, at least about 100,000 nucleic acid molecules, at least about 500,000 nucleic acids, at least about 1,000,000 nucleic acid molecules, at least about 5,000,000 nucleic acid molecules, at least about 10,000,000 nucleic acid molecules, at least about 50,000,000 nucleic acid molecules, at least about 100,000,000 nucleic acid molecules, at least about 250,000,000 nucleic acid molecules and in some cases at least about 1 billion nucleic acid molecules.

In some cases, it may be desirable to incorporate multiple different barcodes within a given partition, either attached to a single or multiple beads within the partition. For example, in some cases, a mixed, but known set of barcode sequences may provide greater assurance of identification in the subsequent processing, e.g., by providing a stronger address or attribution of the barcodes to a given partition, as a duplicate or independent confirmation of the output from a given partition.

The nucleic acid molecules (e.g., oligonucleotides) are releasable from the beads upon the application of a particular stimulus to the beads. In some cases, the stimulus may be a photo-stimulus, e.g., through cleavage of a photo-labile linkage that releases the nucleic acid molecules. In other cases, a thermal stimulus may be used, where elevation of the temperature of the beads environment will result in cleavage of a linkage or other release of the nucleic acid molecules form the beads. In still other cases, a chemical stimulus can be used that cleaves a linkage of the nucleic acid molecules to the beads, or otherwise results in release of the nucleic acid molecules from the beads. In one case, such compositions include the polyacrylamide matrices described above for encapsulation of biological particles, and may be degraded for release of the attached nucleic acid molecules through exposure to a reducing agent, such as DTT.

In some aspects, provided are systems and methods for controlled partitioning. Droplet size may be controlled by adjusting certain geometric features in channel architecture (e.g., microfluidics channel architecture). For example, an expansion angle, width, and/or length of a channel may be adjusted to control droplet size.

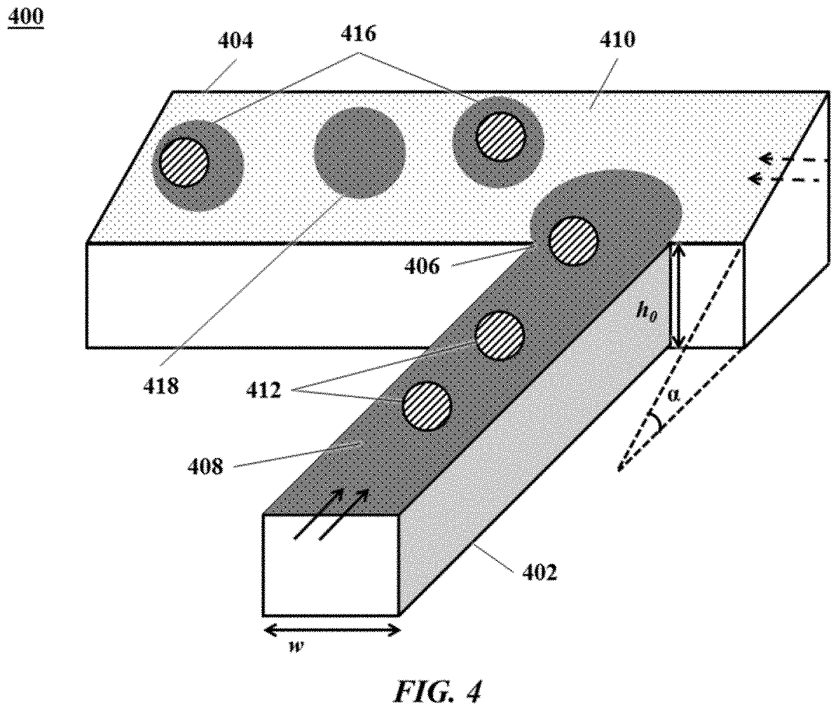

FIG. 4 shows an example of a microfluidic channel structure for the controlled partitioning of beads into discrete droplets. A channel structure 400 can include a channel segment 402 communicating at a channel junction 406 (or intersection) with a reservoir 404. The reservoir 404 can be a chamber. Any reference to “reservoir,” as used herein, can also refer to a “chamber.” In operation, an aqueous fluid 408 that includes suspended beads 412 may be transported along the channel segment 402 into the junction 406 to meet a second fluid 410 that is immiscible with the aqueous fluid 408 in the reservoir 404 to create droplets 416, 418 of the aqueous fluid 408 flowing into the reservoir 404. At the junction 406 where the aqueous fluid 408 and the second fluid 410 meet, droplets can form based on factors such as the hydrodynamic forces at the junction 406, flow rates of the two fluids 408, 410, fluid properties, and certain geometric parameters (e.g., w, h₀, α, etc.) of the channel structure 400. A plurality of droplets can be collected in the reservoir 404 by continuously injecting the aqueous fluid 408 from the channel segment 402 through the junction 406.

A discrete droplet generated may include a bead (e.g., as in occupied droplets 416). Alternatively, a discrete droplet generated may include more than one bead. Alternatively, a discrete droplet generated may not include any beads (e.g., as in unoccupied droplet 418). In some instances, a discrete droplet generated may contain one or more biological particles, as described elsewhere herein. In some instances, a discrete droplet generated may comprise one or more reagents, as described elsewhere herein.

In some instances, the aqueous fluid 408 can have a substantially uniform concentration or frequency of beads 412. The beads 412 can be introduced into the channel segment 402 from a separate channel (not shown in FIG. 4). The frequency of beads 412 in the channel segment 402 may be controlled by controlling the frequency in which the beads 412 are introduced into the channel segment 402 and/or the relative flow rates of the fluids in the channel segment 402 and the separate channel. In some instances, the beads can be introduced into the channel segment 402 from a plurality of different channels, and the frequency controlled accordingly.

In some instances, the aqueous fluid 408 in the channel segment 402 can comprise biological particles (e.g., described with reference to FIGS. 1 and 2). In some instances, the aqueous fluid 408 can have a substantially uniform concentration or frequency of biological particles. As with the beads, the biological particles can be introduced into the channel segment 402 from a separate channel. The frequency or concentration of the biological particles in the aqueous fluid 408 in the channel segment 402 may be controlled by controlling the frequency in which the biological particles are introduced into the channel segment 402 and/or the relative flow rates of the fluids in the channel segment 402 and the separate channel. In some instances, the biological particles can be introduced into the channel segment 402 from a plurality of different channels, and the frequency controlled accordingly. In some instances, a first separate channel can introduce beads and a second separate channel can introduce biological particles into the channel segment 402. The first separate channel introducing the beads may be upstream or downstream of the second separate channel introducing the biological particles.

The second fluid 410 can comprise an oil, such as a fluorinated oil, that includes a fluorosurfactant for stabilizing the resulting droplets, for example, inhibiting subsequent coalescence of the resulting droplets.

In some instances, the second fluid 410 may not be subjected to and/or directed to any flow in or out of the reservoir 404. For example, the second fluid 410 may be substantially stationary in the reservoir 404. In some instances, the second fluid 410 may be subjected to flow within the reservoir 404, but not in or out of the reservoir 404, such as via application of pressure to the reservoir 404 and/or as affected by the incoming flow of the aqueous fluid 408 at the junction 406. Alternatively, the second fluid 410 may be subjected and/or directed to flow in or out of the reservoir 404. For example, the reservoir 404 can be a channel directing the second fluid 410 from upstream to downstream, transporting the generated droplets.

The channel structure 400 at or near the junction 406 may have certain geometric features that at least partly determine the sizes of the droplets formed by the channel structure 400. The channel segment 402 can have a height, h₀ and width, w, at or near the junction 406. By way of example, the channel segment 402 can comprise a rectangular cross-section that leads to a reservoir 404 having a wider cross-section (such as in width or diameter). Alternatively, the cross-section of the channel segment 402 can be other shapes, such as a circular shape, trapezoidal shape, polygonal shape, or any other shapes. The top and bottom walls of the reservoir 404 at or near the junction 406 can be inclined at an expansion angle, α. The expansion angle, α, allows the tongue (portion of the aqueous fluid 408 leaving channel segment 402 at junction 406 and entering the reservoir 404 before droplet formation) to increase in depth and facilitate decrease in curvature of the intermediately formed droplet. Droplet size may decrease with increasing expansion angle. The resulting droplet radius, R_{d}, may be predicted by the following equation for the aforementioned geometric parameters of h₀, w, and α:

$${R}_{d} \approx 0.44 ( 1 + 2.2 \sqrt{tan α} \frac{w}{{h}_{0}} ) \frac{{h}_{0}}{\sqrt{tan α}}$$

By way of example, for a channel structure with w=21 μm, h=21 μm, and α=3°, the predicted droplet size is 121 μm. In another example, for a channel structure with w=25 h=25 μm, and α=5°, the predicted droplet size is 123 μm. In another example, for a channel structure with w=28 μm, h=28 μm, and α=7°, the predicted droplet size is 124 μm.

In some instances, the expansion angle, α, may be between a range of from about 0.5° to about 4°, from about 0.1° to about 10°, or from about 0° to about 90°. For example, the expansion angle can be at least about 0.01°, 0.1°, 0.2°, 0.3°, 0.4°, 0.5°, 0.6°, 0.7°, 0.8°, 0.9°, 1°, 2°, 3°, 4°, 5°, 6°, 7°, 8°, 9°, 10°, 15°, 20°, 25°, 30°, 35°, 40°, 45°, 50°, 55°, 60°, 65°, 70°, 75°, 80°, or higher. In some instances, the expansion angle can be at most about 89°, 88°, 87°, 86°, 85°, 84°, 83°, 82°, 81°, 80°, 75°, 70°, 65°, 60°, 55°, 50°,45°, 40°, 35°, 30°, 25°, 20°, 15°, 10°, 9°, 8°, 7°, 6°, 5°, 4°, 3°, 2°, 1°, 0.1°, 0.01°, or less. In some instances, the width, w, can be between a range of from about 100 micrometers (μm) to about 500 μm. In some instances, the width, w, can be between a range of from about 10 μm to about 200 μm. Alternatively, the width can be less than about 10 μm. Alternatively, the width can be greater than about 500 μm. In some instances, the flow rate of the aqueous fluid 408 entering the junction 406 can be between about 0.04 microliters (μL)/minute (min) and about 40 μL/min. In some instances, the flow rate of the aqueous fluid 408 entering the junction 406 can be between about 0.01 microliters (μL)/minute (min) and about 100 μL/min. Alternatively, the flow rate of the aqueous fluid 408 entering the junction 406 can be less than about 0.01 μL/min. Alternatively, the flow rate of the aqueous fluid 408 entering the junction 406 can be greater than about 40 μL/min, such as 45 μL/min, 50 μL/min, 55 μL/min, 60 μL/min, 65 μL/min, 70 μL/min, 75 μL/min, 80 μL/min, 85 μL/min, 90 μL/min, 95 μL/min, 100 μL/min, 110 μL/min, 120 μL/min, 130 μL/min, 140 μL/min, 150 μL/min, or greater. At lower flow rates, such as flow rates of about less than or equal to 10 microliters/minute, the droplet radius may not be dependent on the flow rate of the aqueous fluid 408 entering the junction 406.

In some instances, at least about 50% of the droplets generated can have uniform size. In some instances, at least about 55%, 60%, 65%, 70%, 75%, 80%, 85%, 90%, 95%, 96%, 97%, 98%, 99%, or greater of the droplets generated can have uniform size. Alternatively, less than about 50% of the droplets generated can have uniform size.

The throughput of droplet generation can be increased by increasing the points of generation, such as increasing the number of junctions (e.g., junction 406) between aqueous fluid 408 channel segments (e.g., channel segment 402) and the reservoir 404. Alternatively or in addition, the throughput of droplet generation can be increased by increasing the flow rate of the aqueous fluid 408 in the channel segment 402.

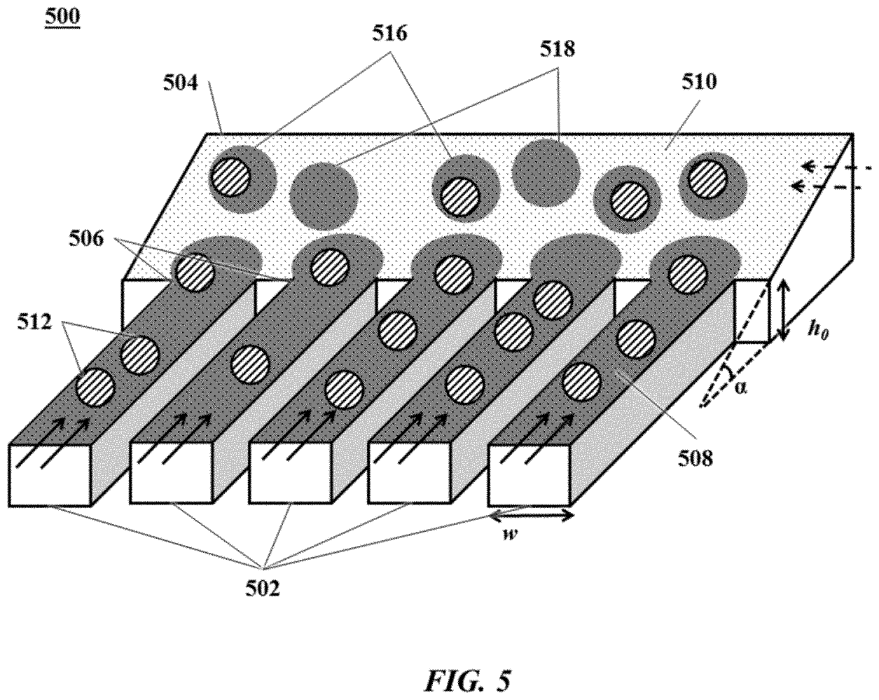

FIG. 5 shows an example of a microfluidic channel structure for increased droplet generation throughput. A microfluidic channel structure 500 can comprise a plurality of channel segments 502 and a reservoir 504. Each of the plurality of channel segments 502 may be in fluid communication with the reservoir 504. The channel structure 500 can comprise a plurality of channel junctions 506 between the plurality of channel segments 502 and the reservoir 504. Each channel junction can be a point of droplet generation. The channel segment 402 from the channel structure 400 in FIG. 4 and any description to the components thereof may correspond to a given channel segment of the plurality of channel segments 502 in channel structure 500 and any description to the corresponding components thereof. The reservoir 404 from the channel structure 400 and any description to the components thereof may correspond to the reservoir 504 from the channel structure 500 and any description to the corresponding components thereof.

Each channel segment of the plurality of channel segments 502 may comprise an aqueous fluid 508 that includes suspended beads 512. The reservoir 504 may comprise a second fluid 510 that is immiscible with the aqueous fluid 508. In some instances, the second fluid 510 may not be subjected to and/or directed to any flow in or out of the reservoir 504. For example, the second fluid 510 may be substantially stationary in the reservoir 504. In some instances, the second fluid 510 may be subjected to flow within the reservoir 504, but not in or out of the reservoir 504, such as via application of pressure to the reservoir 504 and/or as affected by the incoming flow of the aqueous fluid 508 at the junctions. Alternatively, the second fluid 510 may be subjected and/or directed to flow in or out of the reservoir 504. For example, the reservoir 504 can be a channel directing the second fluid 510 from upstream to downstream, transporting the generated droplets.

In operation, the aqueous fluid 508 that includes suspended beads 512 may be transported along the plurality of channel segments 502 into the plurality of junctions 506 to meet the second fluid 510 in the reservoir 504 to create droplets 516, 518. A droplet may form from each channel segment at each corresponding junction with the reservoir 504. At the junction where the aqueous fluid 508 and the second fluid 510 meet, droplets can form based on factors such as the hydrodynamic forces at the junction, flow rates of the two fluids 508, 510, fluid properties, and certain geometric parameters (e.g., w, h₀, α, etc.) of the channel structure 500, as described elsewhere herein. A plurality of droplets can be collected in the reservoir 504 by continuously injecting the aqueous fluid 508 from the plurality of channel segments 502 through the plurality of junctions 506. Throughput may significantly increase with the parallel channel configuration of channel structure 500. For example, a channel structure having five inlet channel segments comprising the aqueous fluid 508 may generate droplets five times as frequently than a channel structure having one inlet channel segment, provided that the fluid flow rate in the channel segments are substantially the same. The fluid flow rate in the different inlet channel segments may or may not be substantially the same. A channel structure may have as many parallel channel segments as is practical and allowed for the size of the reservoir. For example, the channel structure may have at least about 2, 3, 4, 5, 6, 7, 8, 9, 10, 20, 30, 40, 50, 60, 70, 80, 90, 100, 150, 500, 250, 300, 350, 400, 450, 500, 600, 700, 800, 900, 1000, 1500, 5000 or more parallel or substantially parallel channel segments.

The geometric parameters, w, h₀, and α, may or may not be uniform for each of the channel segments in the plurality of channel segments 502. For example, each channel segment may have the same or different widths at or near its respective channel junction with the reservoir 504. For example, each channel segment may have the same or different height at or near its respective channel junction with the reservoir 504. In another example, the reservoir 504 may have the same or different expansion angle at the different channel junctions with the plurality of channel segments 502. When the geometric parameters are uniform, beneficially, droplet size may also be controlled to be uniform even with the increased throughput. In some instances, when it is desirable to have a different distribution of droplet sizes, the geometric parameters for the plurality of channel segments 502 may be varied accordingly.

In some instances, at least about 50% of the droplets generated can have uniform size. In some instances, at least about 55%, 60%, 65%, 70%, 75%, 80%, 85%, 90%, 95%, 96%, 97%, 98%, 99%, or greater of the droplets generated can have uniform size. Alternatively, less than about 50% of the droplets generated can have uniform size.

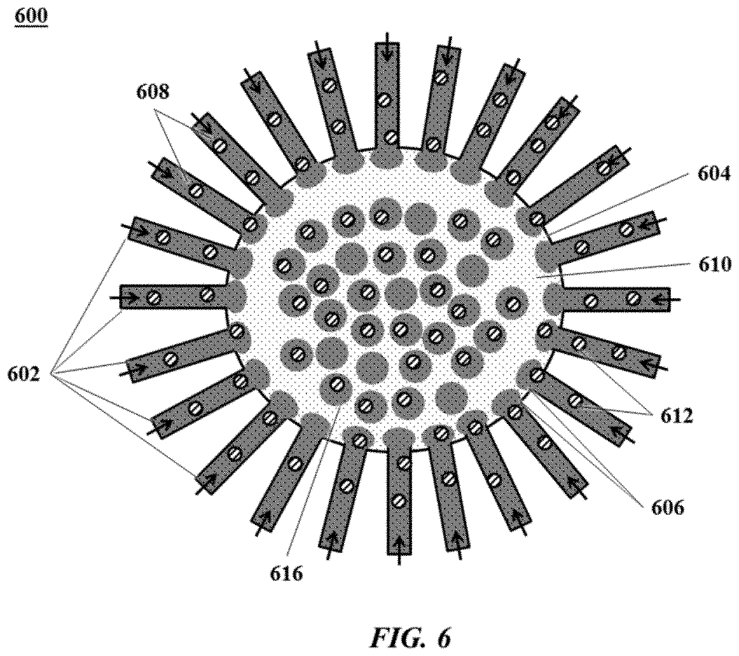

FIG. 6 shows another example of a microfluidic channel structure for increased droplet generation throughput. A microfluidic channel structure 600 can comprise a plurality of channel segments 602 arranged generally circularly around the perimeter of a reservoir 604. Each of the plurality of channel segments 602 may be in fluid communication with the reservoir 604. The channel structure 600 can comprise a plurality of channel junctions 606 between the plurality of channel segments 602 and the reservoir 604. Each channel junction can be a point of droplet generation. The channel segment 402 from the channel structure 400 in FIG. 2 and any description to the components thereof may correspond to a given channel segment of the plurality of channel segments 602 in channel structure 600 and any description to the corresponding components thereof. The reservoir 404 from the channel structure 400 and any description to the components thereof may correspond to the reservoir 604 from the channel structure 600 and any description to the corresponding components thereof.

Each channel segment of the plurality of channel segments 602 may comprise an aqueous fluid 608 that includes suspended beads 612. The reservoir 604 may comprise a second fluid 610 that is immiscible with the aqueous fluid 608. In some instances, the second fluid 610 may not be subjected to and/or directed to any flow in or out of the reservoir 604. For example, the second fluid 610 may be substantially stationary in the reservoir 604. In some instances, the second fluid 610 may be subjected to flow within the reservoir 604, but not in or out of the reservoir 604, such as via application of pressure to the reservoir 604 and/or as affected by the incoming flow of the aqueous fluid 608 at the junctions. Alternatively, the second fluid 610 may be subjected and/or directed to flow in or out of the reservoir 604. For example, the reservoir 604 can be a channel directing the second fluid 610 from upstream to downstream, transporting the generated droplets.

In operation, the aqueous fluid 608 that includes suspended beads 612 may be transported along the plurality of channel segments 602 into the plurality of junctions 606 to meet the second fluid 610 in the reservoir 604 to create a plurality of droplets 616. A droplet may form from each channel segment at each corresponding junction with the reservoir 604. At the junction where the aqueous fluid 608 and the second fluid 610 meet, droplets can form based on factors such as the hydrodynamic forces at the junction, flow rates of the two fluids 608, 610, fluid properties, and certain geometric parameters (e.g., widths and heights of the channel segments 602, expansion angle of the reservoir 604, etc.) of the channel structure 600, as described elsewhere herein. A plurality of droplets can be collected in the reservoir 604 by continuously injecting the aqueous fluid 608 from the plurality of channel segments 602 through the plurality of junctions 606. Throughput may significantly increase with the substantially parallel channel configuration of the channel structure 600. A channel structure may have as many substantially parallel channel segments as is practical and allowed for by the size of the reservoir. For example, the channel structure may have at least about 2, 3, 4, 5, 6, 7, 8, 9, 10, 20, 30, 40, 50, 60, 70, 80, 90, 100, 150, 200, 250, 300, 350, 400, 450, 500, 600, 700, 800, 900, 1000, 1500, 5000 or more parallel or substantially parallel channel segments. The plurality of channel segments may be substantially evenly spaced apart, for example, around an edge or perimeter of the reservoir. Alternatively, the spacing of the plurality of channel segments may be uneven.

The reservoir 604 may have an expansion angle, α (not shown in FIG. 6) at or near each channel junction. Each channel segment of the plurality of channel segments 602 may have a width, w, and α height, h₀, at or near the channel junction. The geometric parameters, w, h₀, and α, may or may not be uniform for each of the channel segments in the plurality of channel segments 602. For example, each channel segment may have the same or different widths at or near its respective channel junction with the reservoir 604. For example, each channel segment may have the same or different height at or near its respective channel junction with the reservoir 604.

The reservoir 604 may have the same or different expansion angle at the different channel junctions with the plurality of channel segments 602. For example, a circular reservoir (as shown in FIG. 6) may have a conical, dome-like, or hemispherical ceiling (e.g., top wall) to provide the same or substantially same expansion angle for each channel segments 602 at or near the plurality of channel junctions 606. When the geometric parameters are uniform, beneficially, resulting droplet size may be controlled to be uniform even with the increased throughput. In some instances, when it is desirable to have a different distribution of droplet sizes, the geometric parameters for the plurality of channel segments 602 may be varied accordingly.

In some instances, at least about 50% of the droplets generated can have uniform size. In some instances, at least about 55%, 60%, 65%, 70%, 75%, 80%, 85%, 90%, 95%, 96%, 97%, 98%, 99%, or greater of the droplets generated can have uniform size. Alternatively, less than about 50% of the droplets generated can have uniform size. The beads and/or biological particle injected into the droplets may or may not have uniform size.

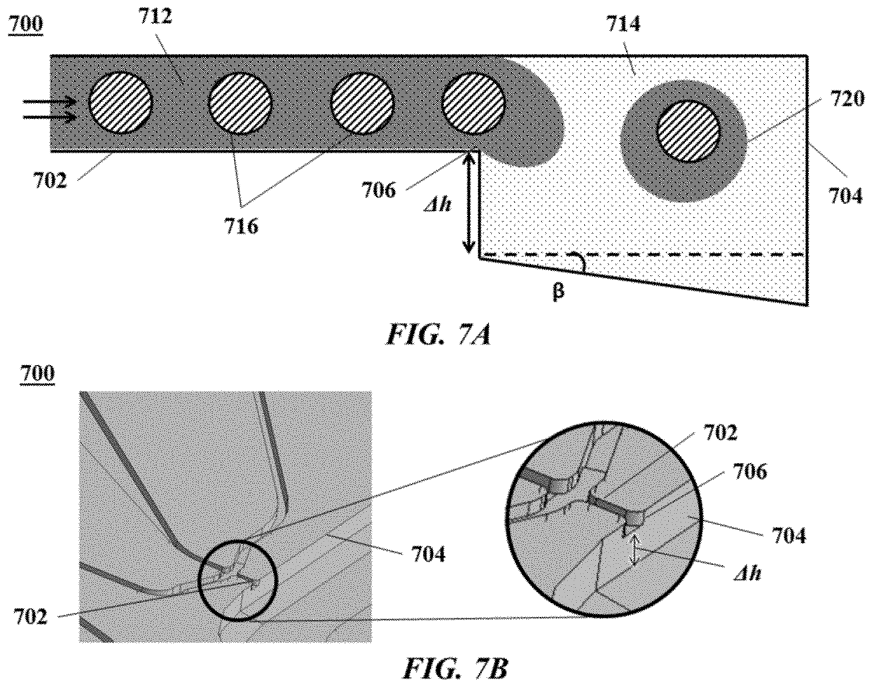

FIG. 7A shows a cross-section view of another example of a microfluidic channel structure with a geometric feature for controlled partitioning. A channel structure 700 can include a channel segment 702 communicating at a channel junction 706 (or intersection) with a reservoir 704. In some instances, the channel structure 700 and one or more of its components can correspond to the channel structure 100 and one or more of its components. FIG. 7B shows a perspective view of the channel structure 700 of FIG. 7A.

An aqueous fluid 712 comprising a plurality of particles 716 may be transported along the channel segment 702 into the junction 706 to meet a second fluid 714 (e.g., oil, etc.) that is immiscible with the aqueous fluid 712 in the reservoir 704 to create droplets 720 of the aqueous fluid 712 flowing into the reservoir 704. At the junction 706 where the aqueous fluid 712 and the second fluid 714 meet, droplets can form based on factors such as the hydrodynamic forces at the junction 706, relative flow rates of the two fluids 712, 714, fluid properties, and certain geometric parameters (e.g., Δh, etc.) of the channel structure 700. A plurality of droplets can be collected in the reservoir 704 by continuously injecting the aqueous fluid 712 from the channel segment 702 at the junction 706.

A discrete droplet generated may comprise one or more particles of the plurality of particles 716. As described elsewhere herein, a particle may be any particle, such as a bead, cell bead, gel bead, biological particle, macromolecular constituents of biological particle, or other particles. Alternatively, a discrete droplet generated may not include any particles.

In some instances, the aqueous fluid 712 can have a substantially uniform concentration or frequency of particles 716. As described elsewhere herein (e.g., with reference to FIG. 4), the particles 716 (e.g., beads) can be introduced into the channel segment 702 from a separate channel (not shown in FIG. 7). The frequency of particles 716 in the channel segment 702 may be controlled by controlling the frequency in which the particles 716 are introduced into the channel segment 702 and/or the relative flow rates of the fluids in the channel segment 702 and the separate channel. In some instances, the particles 716 can be introduced into the channel segment 702 from a plurality of different channels, and the frequency controlled accordingly. In some instances, different particles may be introduced via separate channels. For example, a first separate channel can introduce beads and a second separate channel can introduce biological particles into the channel segment 702. The first separate channel introducing the beads may be upstream or downstream of the second separate channel introducing the biological particles.

In some instances, the second fluid 714 may not be subjected to and/or directed to any flow in or out of the reservoir 704. For example, the second fluid 714 may be substantially stationary in the reservoir 704. In some instances, the second fluid 714 may be subjected to flow within the reservoir 704, but not in or out of the reservoir 704, such as via application of pressure to the reservoir 704 and/or as affected by the incoming flow of the aqueous fluid 712 at the junction 706. Alternatively, the second fluid 714 may be subjected and/or directed to flow in or out of the reservoir 704. For example, the reservoir 704 can be a channel directing the second fluid 714 from upstream to downstream, transporting the generated droplets.

The channel structure 700 at or near the junction 706 may have certain geometric features that at least partly determine the sizes and/or shapes of the droplets formed by the channel structure 700. The channel segment 702 can have a first cross-section height, h₁, and the reservoir 704 can have a second cross-section height, h₂. The first cross-section height, h₁, and the second cross-section height, h₂, may be different, such that at the junction 706, there is a height difference of Δh. The second cross-section height, h₂, may be greater than the first cross-section height, h₁. In some instances, the reservoir may thereafter gradually increase in cross-section height, for example, the more distant it is from the junction 706. In some instances, the cross-section height of the reservoir may increase in accordance with expansion angle, β, at or near the junction 706. The height difference, Δh, and/or expansion angle, β, can allow the tongue (portion of the aqueous fluid 712 leaving channel segment 702 at junction 706 and entering the reservoir 704 before droplet formation) to increase in depth and facilitate decrease in curvature of the intermediately formed droplet. For example, droplet size may decrease with increasing height difference and/or increasing expansion angle.

The height difference, Δh, can be at least about 1 μm. Alternatively, the height difference can be at least about 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 200, 300, 400, 500 μm or more. Alternatively, the height difference can be at most about 500, 400, 300, 200, 100, 90, 80, 70, 60, 50, 45, 40, 35, 30, 25, 20, 19, 18, 17, 16, 15, 14, 13, 12, 11, 10, 9, 8, 7, 6, 5, 4, 3, 2, 1 μm or less. In some instances, the expansion angle, β, may be between a range of from about 0.5° to about 4°, from about 0.1° to about 10°, or from about 0° to about 90°. For example, the expansion angle can be at least about 0.01°, 0.1°, 0.2°, 0.3°, 0.4°, 0.5°, 0.6°, 0.7°, 0.8°, 0.9°, 1°, 2°, 3°, 4°, 5°, 6°, 7°, 8°, 9°, 10°, 15°, 20°, 25°, 30°, 35°, 40°, 45°, 50°, 55°, 60°, 65°, 70°, 75°, 80°, 85°, or higher. In some instances, the expansion angle can be at most about 89°, 88°, 87°, 86°, 85°, 84°, 83°, 82°, 81°, 80°, 75°, 70°, 65°, 60°, 55°, 50°, 45°, 40°, 35°, 30°, 25°, 20°, 15°, 10°, 9°, 8°, 7°, 6°, 5°, 4°, 3°, 2°, 1°, 0.01°, or less.

In some instances, the flow rate of the aqueous fluid 712 entering the junction 706 can be between about 0.04 microliters (μL)/minute (min) and about 40 μL/min. In some instances, the flow rate of the aqueous fluid 712 entering the junction 706 can be between about 0.01 microliters (μL)/minute (min) and about 100 μL/min. Alternatively, the flow rate of the aqueous fluid 712 entering the junction 706 can be less than about 0.01 μL/min. Alternatively, the flow rate of the aqueous fluid 712 entering the junction 706 can be greater than about 40 μL/min, such as 45 μL/min, 50 μL/min, 55 μL/min, 60 μL/min, 65 μL/min, 70 μL/min, 75 μL/min, 80 μL/min, 85 μL/min, 90 μL/min, 95 μL/min, 100 μL/min, 110 μL/min, 120 μL/min, 130 μL/min, 140 μL/min, 150 μL/min, or greater. At lower flow rates, such as flow rates of about less than or equal to 10 microliters/minute, the droplet radius may not be dependent on the flow rate of the aqueous fluid 712 entering the junction 706. The second fluid 714 may be stationary, or substantially stationary, in the reservoir 704. Alternatively, the second fluid 714 may be flowing, such as at the above flow rates described for the aqueous fluid 712.

In some instances, at least about 50% of the droplets generated can have uniform size. In some instances, at least about 55%, 60%, 65%, 70%, 75%, 80%, 85%, 90%, 95%, 96%, 97%, 98%, 99%, or greater of the droplets generated can have uniform size. Alternatively, less than about 50% of the droplets generated can have uniform size.

While FIGS. 7A and 7B illustrate the height difference, Δh, being abrupt at the junction 706 (e.g., a step increase), the height difference may increase gradually (e.g., from about 0 μm to a maximum height difference). Alternatively, the height difference may decrease gradually (e.g., taper) from a maximum height difference. A gradual increase or decrease in height difference, as used herein, may refer to a continuous incremental increase or decrease in height difference, wherein an angle between any one differential segment of a height profile and an immediately adjacent differential segment of the height profile is greater than 90°. For example, at the junction 706, a bottom wall of the channel and a bottom wall of the reservoir can meet at an angle greater than 90°. Alternatively or in addition, a top wall (e.g., ceiling) of the channel and a top wall (e.g., ceiling) of the reservoir can meet an angle greater than 90°. A gradual increase or decrease may be linear or non-linear (e.g., exponential, sinusoidal, etc.). Alternatively or in addition, the height difference may variably increase and/or decrease linearly or non-linearly. While FIGS. 7A and 7B illustrate the expanding reservoir cross-section height as linear (e.g., constant expansion angle, β), the cross-section height may expand non-linearly. For example, the reservoir may be defined at least partially by a dome-like (e.g., hemispherical) shape having variable expansion angles. The cross-section height may expand in any shape.

The channel networks, e.g., as described above or elsewhere herein, can be fluidly coupled to appropriate fluidic components. For example, the inlet channel segments are fluidly coupled to appropriate sources of the materials they are to deliver to a channel junction. These sources may include any of a variety of different fluidic components, from simple reservoirs defined in or connected to a body structure of a microfluidic device, to fluid conduits that deliver fluids from off-device sources, manifolds, fluid flow units (e.g., actuators, pumps, compressors) or the like. Likewise, the outlet channel segment (e.g., channel segment 208, reservoir 604, etc.) may be fluidly coupled to a receiving vessel or conduit for the partitioned cells for subsequent processing. Again, this may be a reservoir defined in the body of a microfluidic device, or it may be a fluidic conduit for delivering the partitioned cells to a subsequent process operation, instrument or component.

The methods and systems described herein may be used to greatly increase the efficiency of single cell applications and/or other applications receiving droplet-based input. For example, following the sorting of occupied cells and/or appropriately-sized cells, subsequent operations that can be performed can include generation of amplification products, purification (e.g., via solid phase reversible immobilization (SPRI)), further processing (e.g., shearing, ligation of functional sequences, and subsequent amplification (e.g., via PCR)). These operations may occur in bulk (e.g., outside the partition). In the case where a partition is a droplet in an emulsion, the emulsion can be broken and the contents of the droplet pooled for additional operations. Additional reagents that may be co-partitioned along with the barcode bearing bead may include oligonucleotides to block ribosomal RNA (rRNA) and nucleases to digest genomic DNA from cells. Alternatively, rRNA removal agents may be applied during additional processing operations. The configuration of the constructs generated by such a method can help minimize (or avoid) sequencing of the poly-T sequence during sequencing and/or sequence the 5′ end of a polynucleotide sequence. The amplification products, for example, first amplification products and/or second amplification products, may be subject to sequencing for sequence analysis. In some cases, amplification may be performed using the Partial Hairpin Amplification for Sequencing (PHASE) method.

A variety of applications require the evaluation of the presence and quantification of different biological particle or organism types within a population of biological particles, including, for example, microbiome analysis and characterization, environmental testing, food safety testing, epidemiological analysis, e.g., in tracing contamination or the like.

### Computer Systems

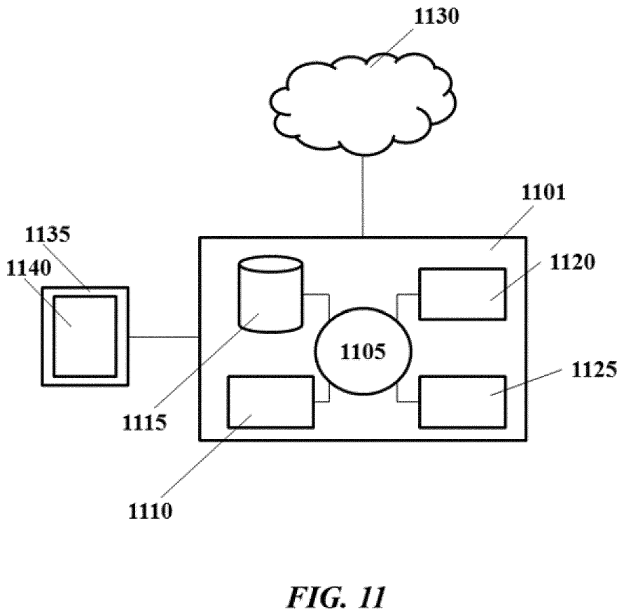

The present disclosure provides computer systems that are programmed to implement methods of the disclosure. FIG. 11 shows a computer system 1101 that is programmed or otherwise configured to, for example, (i) control a microfluidics system (e.g., fluid flow), (ii) sort occupied droplets from unoccupied droplets, (iii) polymerize droplets, (iv) perform sequencing applications, or (v) generate and maintain a library of nucleic acid molecules. The computer system 1101 can regulate various aspects of the present disclosure, such as, for example, fluid flow rates in one or more channels in a microfluidic structure, polymerization application units, etc. The computer system 1101 can be an electronic device of a user or a computer system that is remotely located with respect to the electronic device. The electronic device can be a mobile electronic device.

The computer system 1101 includes a central processing unit (CPU, also “processor” and “computer processor” herein) 1105, which can be a single core or multi core processor, or a plurality of processors for parallel processing. The computer system 1101 also includes memory or memory location 1110 (e.g., random-access memory, read-only memory, flash memory), electronic storage unit 1115 (e.g., hard disk), communication interface 1120 (e.g., network adapter) for communicating with one or more other systems, and peripheral devices 1125, such as cache, other memory, data storage and/or electronic display adapters. The memory 1110, storage unit 1115, interface 1120 and peripheral devices 1125 are in communication with the CPU 1105 through a communication bus (solid lines), such as a motherboard. The storage unit 1115 can be a data storage unit (or data repository) for storing data. The computer system 1101 can be operatively coupled to a computer network (“network”) 1130 with the aid of the communication interface 1120. The network 1130 can be the Internet, an internet and/or extranet, or an intranet and/or extranet that is in communication with the Internet. The network 1130 in some cases is a telecommunication and/or data network. The network 1130 can include one or more computer servers, which can enable distributed computing, such as cloud computing. The network 1130, in some cases with the aid of the computer system 1101, can implement a peer-to-peer network, which may enable devices coupled to the computer system 1101 to behave as a client or a server.

The CPU 1105 can execute a sequence of machine-readable instructions, which can be embodied in a program or software. The instructions may be stored in a memory location, such as the memory 1110. The instructions can be directed to the CPU 1105, which can subsequently program or otherwise configure the CPU 1105 to implement methods of the present disclosure. Examples of operations performed by the CPU 1105 can include fetch, decode, execute, and writeback.

The CPU 1105 can be part of a circuit, such as an integrated circuit. One or more other components of the system 1101 can be included in the circuit. In some cases, the circuit is an application specific integrated circuit (ASIC).

The storage unit 1115 can store files, such as drivers, libraries and saved programs. The storage unit 1115 can store user data, e.g., user preferences and user programs. The computer system 1101 in some cases can include one or more additional data storage units that are external to the computer system 1101, such as located on a remote server that is in communication with the computer system 1101 through an intranet or the Internet.

The computer system 1101 can communicate with one or more remote computer systems through the network 1130. For instance, the computer system 1101 can communicate with a remote computer system of a user (e.g., operator). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1101 via the network 1130.

Methods as described herein can be implemented by way of machine (e.g., computer processor) executable code stored on an electronic storage location of the computer system 1101, such as, for example, on the memory 1110 or electronic storage unit 1115. The machine executable or machine readable code can be provided in the form of software. During use, the code can be executed by the processor 1105. In some cases, the code can be retrieved from the storage unit 1115 and stored on the memory 1110 for ready access by the processor 1105. In some situations, the electronic storage unit 1115 can be precluded, and machine-executable instructions are stored on memory 1110.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 1101, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1101 can include or be in communication with an electronic display 1135 that comprises a user interface (UI) 1140 for providing, for example, results of sequencing analysis, etc. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit 1105. The algorithm can, for example, perform sequencing, etc.

Devices, systems, compositions and methods of the present disclosure may be used for various applications, such as, for example, processing a single analyte (e.g., RNA, DNA, or protein) or multiple analytes (e.g., DNA and RNA, DNA and protein, RNA and protein, or RNA, DNA and protein) form a single cell. For example, a biological particle (e.g., a cell or cell bead) is partitioned in a partition (e.g., droplet), and multiple analytes from the biological particle are processed for subsequent processing. The multiple analytes may be from the single cell. This may enable, for example, simultaneous proteomic, transcriptomic and genomic analysis of the cell.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1-76. (canceled)

77. A method of processing a sample, the method comprising:
(a) providing a biological particle comprising a plurality of ribonucleic acid (RNA) molecules, wherein said plurality of RNA molecules comprises a first set of RNA molecules and a second set of RNA molecules;
(b) co-partitioning said biological particle and an RNA enrichment enzyme in a partition among a plurality of partitions;
(c) in said partition, lysing or permeabilizing said biological particle, thereby providing access to said plurality of RNA molecules of said biological particle; and
(d) digesting RNA molecules of said second set of RNA molecules, thereby increasing a concentration or amount of said first set of RNA molecules relative to said second set of RNA molecules within said partition.

78. The method of claim 77, wherein said RNA enrichment enzyme is an exonuclease.

79. The method of claim 77, wherein said first set of RNA molecules comprises a RNA molecule comprising one or more features selected from the group consisting of a 5′ cap structure, an untranslated region (UTR), a 5′ triphosphate moiety, and a 5′ hydroxyl moiety.

80. The method of claim 77, wherein said second set of RNA molecules comprises a RNA molecule comprising a 5′-monophosphate moiety.

81. The method of claim 77, wherein said second set of RNA molecules comprises a ribosomal RNA molecule or a mitochondrial RNA molecule.

82. The method of claim 77, wherein said first set of RNA molecules comprises a messenger RNA (mRNA) molecule and said second set of RNA molecules does not comprise an mRNA molecule.

83. The method of claim 82, further comprising, in said partition, reverse transcribing said mRNA molecule, thereby generating a complementary deoxyribonucleic acid (cDNA) molecule.

84. The method of claim 77, wherein said partition further comprises a bead comprising a plurality of nucleic acid barcode molecules attached thereto, wherein nucleic acid barcode molecules of said plurality of nucleic acid barcode molecules comprises a common barcode sequence.

85. The method of claim 84, wherein said bead is a gel bead.

86. The method of claim 84, wherein said plurality of nucleic acid barcode molecules is releasably attached to said bead, and wherein, subsequent to (b), said plurality of nucleic acid barcode molecules is released from said bead into said partition.

87. The method of claim 86, wherein said plurality of nucleic acid barcode molecules is released from said bead upon exposure to a stimulus selected from the group consisting of a thermal stimulus, a photo stimulus, and a chemical stimulus.

88. The method of claim 84, wherein said plurality of nucleic acid barcode molecules comprises at least 100,000 nucleic acid barcode molecules.

89. The method of claim 84, wherein said plurality of nucleic acid barcode molecules comprises oligo(dT) sequences.

90. The method of claim 84, further comprising, subsequent to (d):
(i) coupling RNA molecules from said first set of RNA molecules to said bead; and
(ii) generating complementary deoxyribonucleic acid (cDNA) molecules from said RNA molecules.

91. The method of claim 90 further comprising subjecting said cDNA molecules to nucleic acid amplification reactions, thereby generating amplification products of said cDNA molecules.

92. The method of claim 90, further comprising, between (i) and (ii), removing or releasing said bead from said partition.

93. The method of claim 84, wherein each nucleic acid barcode molecule of said plurality of nucleic acid barcode molecules comprises an identifier sequence that is different from identifier sequences associated with other nucleic acid barcode molecules of said plurality of nucleic acid barcode molecules.

94. The method of claim 84, further comprising, subsequent to (d), using RNA molecules of said first set of RNA molecules and said nucleic acid barcode molecules of said plurality of nucleic acid barcode molecules to synthesize barcoded RNA molecules.

95. The method of claim 94, further comprising synthesizing barcoded cDNA molecules by reverse transcribing said barcoded RNA molecules.

96. The method of claim 77, wherein said biological particle is a cell, a cell nucleus, or a cell bead.
